(19) United States
(12) Patent Application Publication
(10) Pub. No.: US 2008/0064689 A1
(43) Pub. Date: Mar. 13, 2008

(54) 3-[4-(6-Pyridin-3-Yl)-3-Phenyl] -5-(1H-1,2,3-Triazol-1-Ylmethyl)-1,3-Oxazolidin-2-Ones as Antibacterial Agents

(75) Inventors: Daniel Robert Carcanague, Arlington, MA (US); Michael Barry Gravestock, Holliston, MA (US)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 11/569,148

(22) PCT Filed: May 24, 2004
(86) PCT No.: PCT/GB05/02040

§ 371 Date: Nov. 15, 2006

(30) Foreign Application Priority Data
May 25, 2004 (GB) 0411594.5
Jan. 5, 2005 (GB) 0500055.9

(51) Int. Cl. A61K 31/4418; A61K 31/445; A61K 31/497; A61K 31/535; A61K 31/54; C07D 413/04; C07D 413/14

(52) U.S. Cl. 514/227.8; 514/236.2; 514/253.1; 514/318; 514/340; 544/112; 544/364; 544/58.2; 544/58.6; 546/194; 546/271.4; 546/272.1

(57) ABSTRACT

Compounds of the formula (I): or a pharmaceutically-acceptable salt or pro-drug thereof: wherein R¹ is selected for example from hydrogen, halogen, optionally substituted methyl; R² and R³ are independently selected from hydrogen, fluoro, chloro and trifluoromethyl; R⁴ and R⁵ are independently selected, for example, from hydrogen, methyl, optionally substituted (2-4C)alkyl, C(O)R⁶ or R⁴ and R⁵ together with the nitrogen to which they are attached form an optionally substituted 5 or 6 membered, saturated or partially unsaturated heterocyclyl ring or an optionally substituted imidazole ring. Methods for making the compounds of formula (I), compositions containing them and their use as antibacterial agents are also described.

The present invention relates to antibiotic compounds and in particular to antibiotic compounds containing substituted oxazolidinone and isoxazoline rings. This invention further relates to processes for their preparation, to intermediates useful in their preparation, to their use as therapeutic agents and to pharmaceutical compositions containing them.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus (MRSA), methicillin resistant coagulase negative staphylococci (MRCNS), penicillin resistant Streptococcus pneumoniae and multiply resistant Enterococcus faecium.

The major clinically effective antibiotic for treatment of such resistant Gram-positive pathogens is vancomycin. Vancomycin is a glycopeptide and is associated with various toxicities including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Certain antibacterial compounds containing an oxazolidinone ring have been described in the art (for example, Walter A. Gregory et al in J. Med. Chem. 1990, 33, 2569-2578 and 1989, 32(8), 1673-81; Chung-Ho Park et al in J. Med. Chem. 1992, 35, 1156-1165). Bacterial resistance to known antibacterial agents may develop, for example, by (i) the evolution of active binding sites in the bacteria rendering a previously active pharmacophore less effective or redundant, and/or (ii) the evolution of means to chemically deactivate a given pharmacophore, and/or (iii) the evolution of efflux pathways. Therefore, there remains an ongoing need to find new antibacterial agents with a favourable pharmacological profile, in particular for compounds containing new pharmacophores.

Our application WO 03/022824 describes a class of bi-aryl antibiotic compounds containing two substituted oxazolidinone and/or isoxazoline rings which has useful activity against Gram-positive pathogens including MRSA and MRCNS and, in particular, against various strains exhibiting resistance to vancomycin and/or linezolid and against E. faecium strains resistant to both aminoglycosides and clinically used β-lactams, but also to fastidious Gram negative strains such as H. influenzae, M. catarrhalis, mycoplasma spp. and chlamydial strains. These compounds thus contain two groups capable of acting as pharmacophores, which may independently bind at pharmacophore binding sites, or alternatively one of the groups may bind at a pharmacophore binding site whilst the other group fulfills a different role in the mechanism of action.

In that patent application, the oxazolidinone and isoxazoline rings each bear a substituent in the 5-position selected from those substituents generally recognised in the art to be suitable for such antibacterial agents, for example methylacetamides (see for example, WO 93/09103), methylamino-linked heterocycles (see for example WO 00/21960) and heterocyclylmethyl groups (see for example WO 01/81350).

Oxazolidinone containing compounds which are mono amine oxidase (MAO) inhibitors are also known (see for example GB 2028306A). Indeed inhibition of MAO is a potential cause of unwanted side effects in oxazolidinone antibacterial agents and thus it is generally desirable that this property is minimised in any potential antibacterial agent (see for example WO 03/072575). In particular, oxazolidinones with amine and ether containing substituents in the 5-position of the oxazolidinone ring have been described as having potent MAO inhibitory activity (see for example, GB 2028306A; J. Pharm Pharmacol, 1983, 161-165; J. Am. Chem. Soc, 111, 8891-8895; and references therein).

We have now unexpectedly discovered that a class of bi-aryl compounds containing one oxazolidinone and one isoxazoline ring, bearing substituted amine sidechains on the isoxazoline and a triazole ring on the oxazolidinone, possess acceptable levels of MAO inhibition whilst having useful antibacterial activity.

Accordingly the present invention provides a compound of the formula (I), or a pharmaceutically-acceptable salt, or pro-drug thereof,

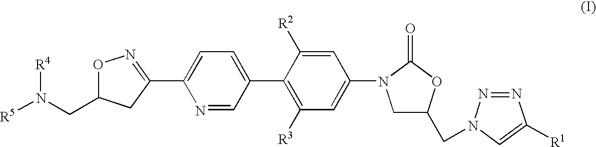

wherein:

### R¹ is selected from hydrogen, halogen, cyano, methyl, cyanomethyl, fluoromethyl, difluoromethyl, trifluoromethyl, methylthio, and (2-4C)alkynyl;

### R² and R³ are independently selected from hydrogen, fluoro, chloro and trifluoromethyl;

R⁴ and R⁵ are independently selected from hydrogen, allyl (optionally substituted on the carbon-carbon double bond by 1, 2 or 3 (1-4C)alkyl groups), methyl, cyanomethyl, carboxymethyl, —CH₂C(O)OR⁶, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], —C(O)R⁶, —C(O)CH₂NR⁶R⁷, —C(O)OR⁶, —C(O)NHR⁶, —C(O)NR⁶R⁷ and —SO₂ NHR⁶;
or R⁴ and R⁵ together with the nitrogen to which they are attached form a 5 or 6 membered, saturated or partially unsaturated heterocyclyl ring and optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl groups;
or R⁴ and R⁵ together with the nitrogen to which they are attached form an imidazole ring, which ring is optionally substituted on an available carbon by 1 or 2 methyl groups;
R⁶ and R⁷ are independently selected from hydrogen, methyl, cyclopropyl (optionally substituted with methyl), carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy);
or R⁶ or R⁷ may form a 4, 5 or 6 membered, carbon-linked saturated heterocyclyl ring, containing 1 or 2 heteroatoms independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom by 1 or 2 (1-4C)alkyl;
or R⁶ and R⁷ together with a nitrogen to which they are attached form a 4, 5 or 6 membered, saturated heterocyclyl ring, optionally containing 1 further heteroatom (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁶ and R⁷ are attached is not thereby quaternised) by 1 or 2 (1-4C)alkyl groups;
provided that R⁴ and R⁵ are not both hydrogen.

In another aspect of the invention there is provided a compound of the formula (I), or a pharmaceutically-acceptable salt, or pro-drug thereof, as hereinbefore defined wherein R⁴ and R⁵ are independently selected from hydrogen, allyl (optionally substituted on the carbon-carbon double bond by 1, 2 or 3 (1-4C)alkyl groups), methyl, cyanomethyl, carboxymethyl, —CH₂C(O)OR⁶, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], —C(O)R⁶, —C(O)CH₂NR⁶R⁷, —C(O)OR⁶, —C(O)NHR⁶, —C(O)NR⁶R⁷ and —SO₂NHR⁶;

or R⁴ and R⁵ together with the nitrogen to which they are attached form a 5 or 6 membered, saturated or partially unsaturated heterocyclyl ring and optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl groups.

In another aspect, the invention relates to compounds of formula (I) as hereinabove defined or to a pharmaceutically acceptable salt.

In another aspect, the invention relates to compounds of formula (I) as hereinabove defined or to a pro-drug thereof. Suitable examples of pro-drugs of compounds of formula (I) are in-vivo hydrolysable esters of compounds of formula (I). Therefore in another aspect, the invention relates to compounds of formula (I) as hereinabove defined or to an in-vivo hydrolysable ester thereof.

In this specification the term ‘alkyl’ includes straight chain and branched structures. For example, (1-4C)alkyl includes propyl and isopropyl. However, references to individual allyl groups such as “propyl” are specific for the straight chain version only, and references to individual branched chain alkyl groups such as “isopropyl” are specific for the branched chain version only. A similar convention applies to other radicals, for example halo(1-4C)alkyl includes 1-bromoethyl and 2-bromoethyl.

In this specification, the terms ‘alkenyl’ and ‘cycloalkenyl’ include all positional and geometrical isomers.

Where optional substituents are chosen from “0, 1, 2 or 3” groups it is to be understood that this definition includes all substituents being chosen from one of the specified groups or the substituents being chosen from two or more of the specified groups. An analogous convention applies to substituents chose from “0, 1 or 2” groups and “1 or 2” groups.

It will be understood that a 4, 5 or 6 membered, saturated or partially unsaturated heterocyclyl ring containing 1 or 2 heteroatoms independently selected from O, N and S (whether or not one of those heteroatoms is a linking N atom), as defined in any definition herein, does not contain any O—O, O—S or S—S bonds.

Within this specification composite terms are used to describe groups comprising more than one functionality such as (1-4C)alkoxy-(1-4C)alkoxy-(1-4C)alkyl. Such terms are to be interpreted in accordance with the meaning which is understood by a person skilled in the art for each component part. For example (1-4C)alkoxy-(1-4C)alkoxy-(1-4C)alkyl includes methoxymethoxymethyl, ethoxymethoxypropyl and propxyethoxymethyl.

It will be understood that where a group is defined such that is optionally substituted by more than one substituent, then substitution is such that chemically stable compounds are formed. For example, a trifluoromethyl group may be allowed but not a trihydroxymethyl group. This convention is applied wherever optional substituents are defined.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

Examples of (1-4C)alkyl include methyl, ethyl, propyl, isopropyl and t-butyl; examples of (2-4C)alkyl include ethyl, propyl, isopropyl and t-butyl; examples of (1-6C)alkyl include methyl, ethyl, propyl, isopropyl, t-butyl, pentyl and hexyl; examples of hydroxy(1-4C)alkyl include hydroxymethyl, 1-hydroxyethyl, 2-hydroxyethyl and 3-hydroxypropyl; examples of hydroxy(2-4C)alkyl include 1-hydroxyethyl, 2-hydroxyethyl, 2-hydroxypropyl, 3-hydroxypropyl, 1-hydroxyisopropyl and 2-hydroxyisopropyl; examples of (1-4C)alkoxycarbonyl include methoxycarbonyl, ethoxycarbonyl and propoxycarbonyl; examples of (2-4C)alkenyl include allyl and vinyl; examples of (2-4C)alkynyl include ethynyl and 2-propynyl; examples of (1-4C)alkanoyl include formyl, acetyl and propionyl; examples of (1-4C)alkoxy include methoxy, ethoxy and propoxy; examples of (1-6C)alkoxy and (1-10C)alkoxy include methoxy, ethoxy, propoxy and pentoxy; examples of (1-4C)alkylthio include methylthio and ethylthio; examples of (1-4C)alkylamino include methylamino, ethylamino and propylamino; examples of di-((1-4C)alkyl)amino include dimethylamino, N-ethyl-N-methylamino, diethylamino, N-methyl-N-propylamino and dipropylamino; examples of halo groups include fluoro, chloro and bromo; examples of (1-4C)alkoxy-(1-4C)alkoxy and (1-6C)alkoxy-(1-6C)alkoxy include methoxymethoxy, 2-methoxyethoxy, 2-ethoxyethoxy and 3-methoxypropoxy; examples of (1-4C)alkanoylamino and (1-6C)alkanoylamino include formamido, acetamido and propionylamino; examples of (1-4C)alkylS(O)_{q}— wherein q is 0, 1 or 2 include methylthio, ethylthio, methylsulfinyl, ethylsulfinyl, methylsulfonyl and ethylsulfonyl; examples of hydroxy-(2-4C)alkoxy include 2-hydroxyethoxy and 3-hydroxypropoxy; examples of (1-6C)alkoxy-(1-6C)alkyl and (1-4C)alkoxy(1-4C)alkyl include methoxymethyl, ethoxymethyl and propoxyethyl; examples of (1-4C)alkylcarbamoyl include methylcarbamoyl and ethylcarbamoyl; examples of di((1-4C)alkyl)carbamoyl include di(methyl)carbamoyl and di(ethyl)carbamoyl; examples of halo groups include fluoro, chloro and bromo; examples of halo(1-4C)alkyl include, halomethyl, 1-haloethyl, 2-haloethyl, and 3-halopropyl; examples of dihalo(1-4C)alkyl include difluoromethyl and dichloromethyl; examples of trihalo(1-4C)alkyl include trifluoromethyl; examples of amino(1-4C)alkyl include aminomethyl, 1-aminoethyl, 2-aminoethyl and 3-aminopropyl; examples of cyano(1-4C)alkyl include cyanomethyl, 1-cyanoethyl, 2-cyanoethyl and 3-cyanopropyl; examples of (1-4C)alkanoyloxy include acetoxy, propanoyloxy; examples of (1-6C)alkanoyloxy include acetoxy, propanoyloxy and tert-butanoyloxy; examples of (1-4C)alkylaminocarbonyl include methylaminocarbonyl and ethylaminocarbonyl; examples of di((1-4C)alkyl)aminocarbonyl include dimethylaminocarbonyl and diethylaminocarbonyl.

A 5- or 6 membered, saturated or partially unsaturated heterocyclyl ring optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group may suitably be a morpholine, piperazine, thiomorpholine (and derivatives thereof wherein the sulphur is oxidised to a S(O) or S(O)₂ group) piperidine, pyrrolidine, dihydropyridine, tetrahydropyridine, dihydroisoxazole, imidazole.

Where optional substituents are listed such substitution is preferably not geminal disubstitution unless stated otherwise. If not stated elsewhere, suitable optional substituents for a particular group are those as stated for similar groups herein.

Suitable pharmaceutically-acceptable salts include acid addition salts such as methanesulfonate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

The compounds of the invention may be administered in the form of a pro-drug which is broken down in the human or animal body to give a compound of the invention. A prodrug may be used to alter or improve the physical and/or pharmacokinetic profile of the parent compound and can be formed when the parent compound contains a suitable group or substituent which can be derivatised to form a prodrug. Examples of pro-drugs include in-vivo hydrolysable esters of a compound of the invention or a pharmaceutically-acceptable salt thereof. Further examples of pro-drugs include in-vivo hydrolysable amides of a compound of the invention or a pharmaceutically-acceptable salt thereof.

Various forms of prodrugs are known in the art, for examples see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

Suitable pro-drugs for pyridine or triazole derivatives include acyloxymethyl pyridinium or triazolium salts eg halides; for example a pro-drug such as:

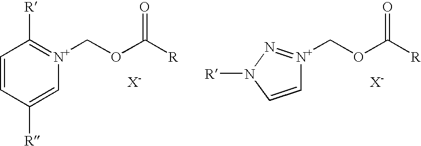

### (Ref: T. Yamazaki et al. 42^{nd} Interscience Conference on Antimicrobial Agents and Chemotherapy, San Diego, 2002; Abstract F820).

Suitable pro-drugs of hydroxyl groups are acyl esters of acetal-carbonate esters of formula RCOOC(R,R′)OCO—, where R is (1-4C)alkyl and R′ is (1-4C)alkyl or H. Further suitable prodrugs are carbonate and carabamate esters RCOO— and RNHCOO—.

An in-vivo hydrolysable ester of a compound of the invention or a pharmaceutically-acceptable salt thereof containing a carboxy or hydroxy group is, for example, a pharmaceutically-acceptable ester which is hydrolysed in the human or animal body to produce the parent alcohol.

Suitable pharmaceutically-acceptable esters for carboxy include (1-6C)alkoxymethyl esters for example methoxymethyl, (1-6C)alkanoyloxymethyl esters for example pivaloyloxymethyl, phthalidyl esters, (3-8C)cycloalkoxycarbonyloxy(1-6C)alkyl esters for example 1-cyclohexylcarbonyloxyethyl; 1,3-dioxolan-2-onylmethyl esters for example 5-methyl-1,3-dioxolan-2-ylmethyl; and (1-6C)alkoxycarbonyloxyethyl esters for example 1-methoxycarbonyloxyethyl and may be formed at any carboxy group in the compounds of this invention.

An in-vivo hydrolysable ester of a compound of the invention or a pharmaceutically-acceptable salt thereof containing a hydroxy group or groups includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxymethoxy. A selection of in-vivo hydrolysable ester forming groups for hydroxy include (1-10C)alkanoyl (for example (1-4C)alkanoyl), benzoyl, phenylacetyl and substituted benzoyl and phenylacetyl, (1-10C)alkoxycarbonyl (to give alkyl carbonate esters), di-(1-4C)alkylcarbamoyl and N-(di-(1-4C)alkylaminoethyl)-N-(1-4C)alkylcarbamoyl (to give carbamates), di-(1-4C)alkylaminoacetyl, carboxy(2-5C)alkylcarbonyl and carboxyacetyl. Examples of ring substituents on phenylacetyl and benzoyl include chloromethyl or aminomethyl, (1-4C)alkylaminomethyl and di-((1-4C)alkyl)aminomethyl, and morpholino or piperazino linked from a ring nitrogen atom via a methylene linking group to the 3- or 4-position of the benzoyl ring. Other interesting in-vivo hydrolysable esters include, for example, R^{A}C(O)O(1-6C)alkyl-CO— (wherein R^{A} is for example, optionally substituted benzyloxy-(1-4C)alkyl, or optionally substituted phenyl; suitable substituents on a phenyl group in such esters include, for example, 4-(1-4C)piperazino-(1-4C)alkyl, piperazino-(1-4C)alkyl and morpholino-(1-4C)alkyl.

Further suitable in-vivo hydrolysable esters are those formed from amino acids. For examples, esters formed by reaction of a hydroxy group of a compound with the carboxylic acid of an amino acid. By the term “amino acid” herein we mean any α- or other amino substituted acid, naturally occurring or otherwise ie. non-naturally occurring, and derivatives thereof such as those formed by substitution (for example by alkylation on the nitrogen of the amino group). The use of either a natural or a non-natural amino acid represent particular and independent aspects of the invention. Examples of suitable α-amino acids and derivatives thereof, are valine, leucine, iso-leucine, N-methyl isoleucine, N-tert-butyl-isoleucine, lysine, glycine, N-methylglycine, N,N-dimethyl glycine, alanine, glutamine, asparagine, proline, and phenylalanine. In one embodiment, preferred amino acids are naturally occurring α-amino acids and N-alkylated derivatives thereof.

The use of amino acids having neutral and/or basic side chains represent particular and independent aspects of the invention.

Suitable in-vivo hydrolysable esters of a compound of the formula (I) are described as follows. For example, a 1,2-diol may be cyclised to form a cyclic ester of formula (PD1) or a pyrophosphate of formula (PD2), and a 1,3-diol may be cyclised to form a cyclic ester of the formula (PD3):

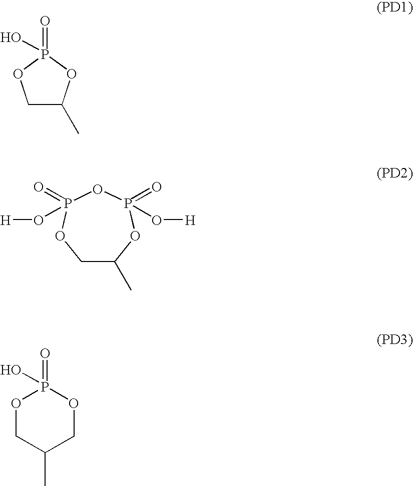

Esters of compounds of formula (I) wherein the HO— function/s in (PD1), (PD2) and (PD3) are protected by (1-4C)alkyl, phenyl or benzyl are useful intermediates for the reparation of such pro-rugs.

Further in-vivo hydrolysable esters include phosphoramidic esters, and also compounds of invention in which any free hydroxy group independently forms a phosphoryl (npd is 1) or phosphiryl (npd is 0) ester of the formula (PD4):

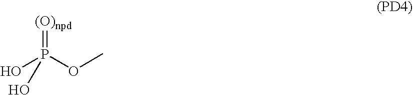

For the avoidance of doubt, phosphono is —P(O)(OH)₂; (1-4C)alkoxy(hydroxy)-phosphoryl is a mono-(1-4C)alkoxy derivative of —O—P(O)(OH)₂; and di-(1-4C)alkoxyphosphoryl is a di-(1-4C)alkoxy derivative of —O—P(O)(OH)₂.

Useful intermediates for the preparation of such esters include compounds containing a group/s of formula (PD4) in which either or both of the —OH groups in (PD1) is independently protected by (1-4C)alkyl (such compounds also being interesting compounds in their own right), phenyl or phenyl-(1-4C)alkyl (such phenyl groups being optionally substituted by 1 or 2 groups independently selected from (1-4C)alkyl, nitro, halo and (1-4C)alkoxy).

Thus, prodrugs containing groups such as (PD1), (PD2), (PD3) and (PD4) may be prepared by reaction of a compound of invention containing suitable hydroxy group/s with a suitably protected phosphorylating agent (for example, containing a chloro or dialkylamino leaving group), followed by oxidation (if necessary) and deprotection.

Other suitable prodrugs include phosphonooxymethyl ethers and their salts, for example a prodrug of R—OH such as:

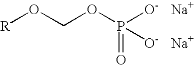

When a compound of invention contains a number of free hydroxy group, those groups not being converted into a prodrug functionality may be protected (for example, using a t-butyl-dimethylsilyl group), and later deprotected. Also, enzymatic methods may be used to selectively phosphorylate or dephosphorylate alcohol functionalities.

Examples of pro-drugs for an amino group include in-vivo hydrolysable amides or a pharmaceutically-acceptable salt thereof. Suitable in-vivo hydrolysable groups include N-carbomethoxy and N-acetyl. Such amides may formed by reaction of an amino (or alkylamino) group with an activated acyl derivative such as an activated ester or an acid chloride, for example, (1-6C)alkanoylchlorides (such as tBuCOCl or acetyl chloride), or substituted derivatives thereof.

A suitable value for an in-vivo hydrolysable amide of a compound of the formula (I) containing a carboxy group is, for example, a N—C₁₋₆alkyl or N,N-di-C₁₋₆alkyl amide such as NV-methyl, N-ethyl, N-propyl, N,N-dimethyl, N-ethyl-N-methyl or N,N-diethyl amide. Further suitable values for in-vivo hydrolysable amides of a compound of the formula (I) containing an amine or carboxy group are in-vivo hydrolysable amides formed by reaction with amino-acids, as defined and described herein for in-vivo hydrolysable esters.

Where pharmaceutically-acceptable salts of an in-vivo hydrolysable ester or amide may be formed this is achieved by conventional techniques. Thus, for example, compounds containing a group of formula (PD1), (PD2), (PD3) and/or (PD4) may ionise (partially or fully) to form salts with an appropriate number of counter-ions. Thus, by way of example, if an in-vivo hydrolysable ester prodrug of a compound of invention contains two (PD4) groups, there are four HO—P— functionalities present in the overall molecule, each of which may form an appropriate salt (i.e. the overall molecule may form, for example, a mono-, di-, tri- or tetra-sodium salt).

In one aspect, suitable pro-drugs of the invention are in-vivo hydrolysable esters such as (1-4C)alkyl esters; (1-4C)alkyl esters substituted with (1-4C)alkoxy, (1-4C)alkoxy(1-4C)alkoxy, carboxy, (1-4C)alkyl esters, amino, (1-4C)alkylamino, di(1-4C)alkylamino, tri(1-4C)alkylamino (thereby containing a quaternised nitrogen atom), aminocarbonyl, carbamates, amides or heterocyclyl groups (for example, an ester formed by reaction of a hydroxy group in R⁴ or R⁵ with methoxy acetic acid, methoxypropionic acid, adipic acid momethylester, 4-dimethylaminobutanoic acid, 2-methylaminobutanoic acid, 5-amino pentanoic acid, β-alanine, N,N-diethylalanine, valine, leucine, iso-leucine, N-methyl isoleucine, N-tert-butyl-isoleucine, lysine, glycine, N,N-dimethyl glycine, alanine, sarcosine, glutamine, asparagine, proline, phenylalanine, nicotinic acid, nicotinic acid —N-oxide, pyrimidine-carboxylic acid (for example pyrimidine-5-carboxylic acid), pyrazine-carboxylic acid (for example pyrazine-2-carboxylic acid), or piperidine-4-carboxylic acid); (3-6C)cycloalkyl esters (optionally substituted by a (1-4C)alkoxycarbonyl, alkoxy or carboxy group); carbonates (for example (1-4C)alkylcarbonates and such carbonates substituted by (1-4C)alkoxy or di(1-4C)alkyl)amino); sulfates; phosphates and phosphate esters; and carbamates (see for example Example 10); and pharmaceutically acceptable salts thereof.

Further suitable pro-drugs are those formed by reaction of a hydroxy group in R⁴ or R⁵ with carbonates, particularly alkoxysubstituted alkyl carbonates such as methoxypropylcarbonate.

Further suitable pro-drugs are esters formed by reaction of a hydroxy group in R⁴ or R⁵ with methoxy acetic acid, methoxypropionic acid, adipic acid momethylester, 4-dimethylaminobutanoic acid, 2-methylaminobutanoic acid, 5-amino pentanoic acid, β-alanine, N,N-diethylalanine, valine, leucine, iso-leucine, N-methyl isoleucine, N-tert-butyl-isoleucine, lysine, glycine, N,N-dimethyl glycine, alanine, sarcosine, glutamine, asparagine, proline, phenylalanine, nicotinic acid, nicotinic acid —N-oxide, pyrimidine-5-carboxylic acid, pyrazine-2-carboxylic acid, or piperidine-4-carboxylic acid, 2-carboxy-cyclohexane-1-carboxylic acid; and pharmaceutically acceptable salts thereof.

Particular compounds of the invention are in-vivo hydrolysable esters formed from amino acids, and pharmaceutically acceptable salts thereof.

Further particular compounds of the invention are in-vivo hydrolysable esters formed from 4-dimethylaminobutanoic acid, 2-methylaminobutanoic acid, 5-amino pentanoic acid, β-alanine, N,N-diethylalanine, valine, leucine, iso-leucine, N-methyl isoleucine, N-tert-butyl-isoleucine, lysine, glycine, N,N-dimethyl glycine, alanine, sarcosine, glutamine, asparagine, proline, phenylalanine; and pharmaceutically acceptable salts thereof.

Further particular compounds of the invention are in-vivo hydrolysable esters formed from valine, leucine, iso-leucine, N-methyl isoleucine, N-tert-butyl-isoleucine, lysine, glycine, N,N-dimethyl glycine, alanine, sarcosine, glutamine, asparagine, proline and phenylalanine; and pharmaceutically acceptable salts thereof.

The compounds of the present invention have a chiral centre at the C-5 positions of the oxazolidinone and isoxazoline rings. The pharmaceutically active diastereomer is of the formula (Ia):

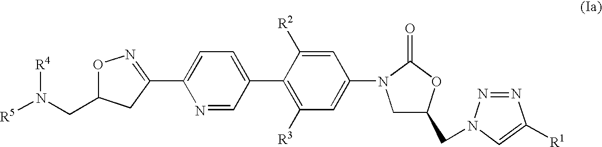

In one aspect a preferred diastereomer is of formula (Ib). In another aspect a preferred diastereomer is of formula (Ic).

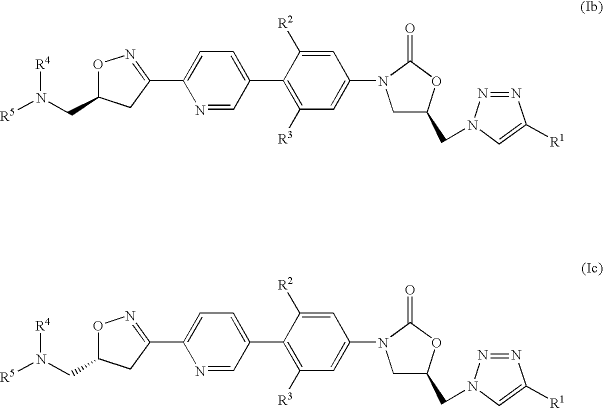

If a mixture of epimers on the oxazolidinone chiral center is used, a larger amount (depending upon the ratio of the diastereoisomers) will be required to achieve the same effect as the same weight of the pharmaceutically active enantiomer.

Furthermore, some compounds of the invention may have other chiral centres, for example on substituent R⁴. It is to be understood that the invention encompasses all such optical and diastereoisomers, and racemic mixtures, that possess antibacterial activity. It is well known in the art how to prepare optically-active forms (for example by resolution of the racemic form by recrystallisation techniques, by chiral synthesis, by enzymatic resolution, by biotransformation or by chromatographic separation) and how to determine antibacterial activity as described hereinafter.

The invention relates to all tautomeric forms of the compounds of the invention that possess antibacterial activity.

It is also to be understood that certain compounds of the invention can exist in solvated as well as unsolvated forms such as, for example, hydrated forms. It is to be understood that the invention encompasses all such solvated forms which possess antibacterial activity.

It is also to be understood that certain compounds of the invention may exhibit polymorphism, and that the invention encompasses all such forms which possess antibacterial activity.

As stated before, we have discovered a range of compounds that generally have good activity against a broad range of Gram-positive pathogens including organisms known to be resistant to most commonly used antibiotics, together with activity against fastidious Gram negative pathogens such as H. influenzae, M. catarrhalis, Mycoplasma and Chlamydia strains. The following compounds possess preferred pharmaceutical and/or physical and/or pharmacokinetic properties, for example solubility and/or bioavailability.

The compounds of the invention generally possess favourable solubility and/or bioavailability due to the basicity of the amine side chain and the nature of the substituents R⁴ and R⁵ leading to ionised or partially ionised species at physiological pH.

It will be appreciated that parameters such as solubility may be measured by any suitable method known in the art.

In one embodiment of the invention are provided compounds of formula (I), in an alternative embodiment are provided pharmaceutically-acceptable salts of compounds of formula (I), in a further alternative embodiment are provided in-vivo hydrolysable esters of compounds of formula (I), and in a further alternative embodiment are provided pharmaceutically-acceptable salts of in-vivo hydrolysable esters of compounds of formula (I). In a further aspect there is provided in-vivo hydrolysable amides of compounds of formula (I).

In one aspect, R¹ is selected from hydrogen, halogen, cyano, methyl, cyanomethyl, fluoromethyl, difluoromethyl, trifluoromethyl, ethynyl and propynyl.

In another aspect, R¹ is selected from hydrogen, chloro, bromo, methyl and fluoromethyl.

In another aspect, R¹ is hydrogen.

In one aspect, R² and R³ are independently hydrogen or fluoro.

In another aspect R² and R³ are both hydrogen.

In another aspect one R² and R³ is hydrogen and the other is fluorine.

In one aspect, R⁴ or R⁵ is hydrogen and the other is selected from any of the values for R⁴ and R⁵ hereinbefore or hereinafter.

In one aspect, R⁴ or R⁵ is methyl and the other is selected from any of the values for R⁴ and R⁵ hereinbefore or hereinafter.

In one embodiment R⁴ and R⁵ are independently selected from hydrogen, allyl (optionally substituted on the carbon-carbon double bond by 1, 2 or 3 (1-4C)alkyl groups), methyl, cyanomethyl, carboxymethyl, —CH₂C(O)OR⁶, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], —C(O)R⁶, —C(O)CH₂NR⁶R⁷, —C(O)OR⁶, —C(O)NHR⁶, —C(O)NR⁶R⁷ and —SO₂NHR⁶, provided that R⁴ and R⁵ are not both hydrogen).

In one aspect, R⁴ and R⁵ are independently selected from hydrogen, methyl, carboxymethyl, —CH₂C(O)OR⁶, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], —C(O)R⁶, —C(O)CH₂NR⁶R⁷, —C(O)OR⁶, —C(O)NHR⁶, —C(O)NR⁶R⁷ and —SO₂NHR⁶ (provided that R⁴ and R⁵ are not both hydrogen).

In a further aspect, R⁴ and R⁵ are independently selected from hydrogen, methyl, carboxymethyl, —CH₂C(O)OR⁶, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], —C(O)R⁶ and —C(O)CH₂NR⁶R⁷.

In one aspect, R⁴ and R⁵ are independently selected from hydrogen, methyl, carboxymethyl, —CH₂C(O)OR⁶, —CH₂C(O)NR⁶R⁷ and (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], (provided that R⁴ and R⁵ are not both hydrogen).

In another aspect, one of R⁴ and R⁵ is hydrogen or methyl, and the other is selected from carboxymethyl, —CH₂C(O)OR⁶, —CH₂C(O)NR⁶R⁷ and (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶].

In another aspect, one of R⁴ and R⁵ is hydrogen or methyl, and the other is selected from carboxymethyl, —CH₂C(O)OR⁶, —CH₂C(O)NR⁶R⁷ and (2-4C)alkyl [substituted by 1 or 2 substituents independently selected from hydroxy, (1-4C)alkoxy, (1-4C)alkoxy(1-4C)alkoxy, hydroxy(2-4C)alkoxy, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶].

In another aspect, one of R⁴ and R⁵ is hydrogen or methyl, and the other is selected from carboxymethyl, —CH₂C(O)OR⁶, —CH₂C(O)NR⁶R⁷ and (2-4C)alkyl [substituted by 1 or 2 substituents independently selected from hydroxy, (1-4C)alkoxy, (1-4C)alkoxy(1-4C)alkoxy, hydroxy(2-4C)alkoxy, —C(O)OR⁶, —OC(O)R⁶, carboxy, —C(O)NR⁶R⁷, —NR⁶R⁷ and —NHC(O)R⁶].

In another aspect, one of R⁴ and R⁵ is hydrogen or methyl, and the other is selected from —C(O)R⁶, —C(O)CH₂NR⁶R⁷, —C(O)OR⁶, —C(O)NR⁶R⁷ and —SO₂NHR⁶.

In another aspect, one of R⁴ and R⁵ is hydrogen or methyl, and the other is selected from —C(O)R⁶, —C(O)CH₂NR⁶R⁷, —C(O)OR⁶ and —C(O)NR⁶R⁷.

In another aspect, one of R⁴ and R⁵ is hydrogen or methyl, and the other is selected from —C(O)R⁶, —C(O)CH₂NR⁶R⁷ and —SO₂NHR⁶.

In another aspect, one of R⁴ and R⁵ is hydrogen or methyl, and the other is selected from —C(O)R⁶ and —C(O)CH₂NR⁶R⁷.

In another aspect, R⁴ and R⁵ are independently selected from hydrogen, methyl, carboxymethyl, —CH₂C(O)OR⁶, (2-4C)alkyl [optionally substituted by 1 or 2 hydroxy], —C(O)R⁶ and —C(O)CH₂NR⁶R⁷; or

R⁴ and R⁵ together with the nitrogen to which they are attached form a morpholine, piperazine, N-methylpiperazine, thiomorpholine (and derivatives thereof wherein the sulfur is oxidised to an S(O) or S(O)₂ group), piperidine and pyrrolidine ring.

In another aspect, R⁴ and R⁵ are independently selected from hydrogen, allyl (optionally substituted on the carbon-carbon double bond by 1, 2 or 3 (1-4C)alkyl groups), methyl, cyanomethyl, carboxymethyl, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], —C(O)R⁶, and —SO₂NHR⁶; provided that R⁴ and R⁵ are not both hydrogen or both —C(O)R⁶ and when R⁴ or R⁵ is —C(O)R⁶ then R⁶ is selected from cyclopropyl (optionally substituted with methyl), carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy);

or R⁶ may form a 4, 5 or 6 membered, carbon-linked saturated heterocyclyl ring, containing 1 or 2 heteroatoms independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom by 1 or 2 (1-4C)alkyl;

In a further aspect, R⁴ and R⁵ together with the nitrogen to which they are attached form a 5 or 6 membered, saturated or partially unsaturated heterocyclyl ring and optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl groups.

In a further aspect, R⁴ and R⁵ together with the nitrogen to which they are attached form a 5 or 6 membered, saturated heterocyclyl ring and optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl groups. Suitably such optional substituents are 1 or 2 methyl groups.

Suitable values for such a ring comprising R⁴ and R⁵ together with the nitrogen to which they are attached are morpholine, piperazine, N-methylpiperazine, thiomorpholine (and derivatives thereof wherein the sulfur is oxidised to an S(O) or S(O)₂ group), piperidine, pyrrolidine and tetrahydropyridine.

Further suitable values for such a ring comprising R⁴ and R⁵ together with the nitrogen to which they are attached are morpholine, piperazine, N-methylpiperazine, thiomorpholine (and derivatives thereof wherein the sulfur is oxidised to an S(O) or S(O)₂ group), piperidine and pyrrolidine.

Further suitable values for such a ring comprising R⁴ and R⁵ together with the nitrogen to which they are attached are piperazine, N-methylpiperazine, piperidine, pyrrolidine and tetrahydropyridine.

Further suitable values for such a ring comprising R⁴ and R⁵ together with the nitrogen to which they are attached are thiomorpholine and derivatives thereof wherein the sulfur is oxidised to an S(O) or S(O)₂ group. A further suitable value is morpholine.

In another aspect, R⁴ and R⁵ together with the nitrogen to which they are attached form a 6 membered mono-unsaturated ring such as tetrahydropyridine.

In another aspect R⁴ and R⁵ together with the nitrogen to which they are attached form an imidazole ring, which ring is optionally substituted on an available carbon by 1 or 2 methyl groups.

In one aspect R⁶ and R⁷ are independently selected from hydrogen, methyl, cyclopropyl (optionally substituted with methyl), carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy).

In another aspect, R⁶ and R⁷ are independently selected from hydrogen, methyl, carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a, (1-4C)alkylamino or di-(1-4C)alkylamino group may optionally be substituted on the (1-4C)alkyl chain with carboxy).

In another aspect, R⁶ and R⁷ are independently selected from hydrogen and (1-4C)alkyl.

In another aspect, R⁶ and R⁷ are independently selected from hydrogen, carboxymethyl and (2-4C)alkyl (substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy).

In another aspect, R⁶ and R⁷ are independently selected from hydrogen, carboxymethyl and (2-4C)alkyl (substituted by 1 or 2 substituents independently selected from amino, methylamino, dimethylamino, carboxy, (1-4C)alkoxy and hydroxy; wherein a methylamino or dimethylamino group may optionally be substituted on the methyl group with carboxy).

In another aspect, R⁶ and R⁷ are independently selected from hydrogen, carboxymethyl and (2-4C)alkyl (substituted by 1 or 2 substituents independently selected from carboxy, (1-4C)alkoxy and hydroxy).

In another aspect R⁶ or R⁷ form a 4, 5 or 6 membered, carbon-linked saturated heterocyclyl ring, containing 1 or 2 heteroatoms independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom by 1 or 2 (1-4C)alkyl. In particular this definition of R⁶ applies when R⁴ is —C(O)R⁶. Particular values for R⁶ or R⁷ as such a ring are azetidine, pyrrolidine and piperidine. Further particular values for R⁶ or R⁷ as such a ring are azetidinone, pyrrolidone and piperidone.

In a further aspect, R⁶ and R⁷ together with a nitrogen to which they are attached form a 4, 5 or 6 membered, saturated heterocyclyl ring, optionally containing 1 further heteroatom (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁶ and R⁷ are attached is not thereby quaternised) by 1 or 2 (1-4C)alkyl groups;

Suitable values for such a ring comprising R⁶ and R⁷ together with the nitrogen to which they are attached are azetidine, morpholine, piperazine, N-methylpiperazine, thiomorpholine (and derivatives thereof wherein the sulfur is oxidised to an S(O) or S(O)₂ group), piperidine and pyrrolidine.

Suitable values for optional substituents on a ring comprising R⁶ and R⁷ together with the nitrogen to which they are attached are 1 or 2 methyl groups.

In another aspect, when R⁶ or R⁷ is (2-4C)alkyl, then the alkyl group is substituted with 1 substituent selected from the substituents for this group in any aspect hereinbefore or hereinafter. In a further aspect, when R⁶ or R⁷ is (2-4C)alkyl, then the alkyl group is substituted with 2 substituents selected from the substituents for this group in any aspect hereinbefore or hereinafter.

In one aspect, when R⁴ or R⁵ is —C(O)NHR⁶ or —C(O)OR⁶ then R⁶ is selected from carboxymethyl and (2-4C)alkyl (substituted by a substituent selected from amino, (1-4C)alkylamino, di-(1-4C)alkylamino, carboxy and hydroxy).

In another aspect, when R⁴ or R⁵ is —C(O)NR⁶R⁷ then R⁶ and R⁷ together with the nitrogen to which they are attached may not form a pyrrolidine, piperidine or morpholine ring.

In another aspect, when R⁴ or R⁵ is —C(O)NR⁶R⁷ then R⁶ and R⁷ together with the nitrogen to which they are attached may not form an unsubstituted pyrrolidine, piperidine or morpholine ring.

In a preferred aspect of the invention, the compound of formula (I) is a compound of the formula (Ia).

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ and R⁵ are independently selected from hydrogen, methyl, carboxymethyl, —CH₂C(O)OR⁶, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], —C(O)R⁶, —C(O)CH₂NR⁶R⁷, —C(O)OR⁶, —C(O)NHR⁶, —C(O)NR⁶R⁷ and —SO₂NHR⁶ (provided that R⁴ and R⁵ are not both hydrogen);

R⁶ and R⁷ are independently selected from hydrogen, methyl, carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ and R⁵ are independently selected from hydrogen, methyl, carboxymethyl, —CH₂C(O)OR⁶, —CH₂C(O)NR⁶R⁷ and (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], provided that R⁴ and R⁵ are not both hydrogen);

R⁶ and R⁷ are independently selected from hydrogen, methyl, carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

one of R⁴ and R⁵ is hydrogen or methyl, and the other is selected from carboxymethyl, —CH₂C(O)OR⁶, —CH₂C(O)NR⁶R⁷ and (2-4C)alkyl [substituted by 1 or 2 substituents independently selected from hydroxy, (1-4C)alkoxy, (1-4C)alkoxy(1-4C)alkoxy, hydroxy(2-4C)alkoxy, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶];

R⁶ and R⁷ are independently selected from hydrogen, methyl, carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

one of R⁴ and R⁵ is hydrogen or methyl, and the other is selected from —C(O)R⁶, —C(O)CH₂NR⁶R⁷, —C(O)OR⁶, —C(O)NR⁶R⁷ and —SO₂NHR⁶;

R⁶ and R⁷ are independently selected from hydrogen, methyl, carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ and R⁵ together with the nitrogen to which they are attached form a 5 or 6 membered, saturated or partially unsaturated heterocyclyl ring and optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl groups;

R⁶ and R⁷ are independently selected from hydrogen, methyl, carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ and R⁵ are independently selected from hydrogen, methyl, carboxymethyl, —CH₂C(O)OR⁶, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)R⁶, —S(O)₂NR⁶R⁷, —NR⁶R⁷, —NHC(O)R⁶ and —NHS(O)₂R⁶], —C(O)R⁶, —C(O)CH₂NR⁶R⁷, —C(O)OR⁶, —C(O)NHR⁶, —C(O)NR⁶R⁷ and —SO₂NHR⁶ (provided that R⁴ and R⁵ are not both hydrogen);

R⁶ and R⁷ together with the nitrogen to which they are attached form an azetidine, morpholine, piperazine, N-methylpiperazine, thiomorpholine (or derivatives thereof wherein the sulfur is oxidised to an S(O) or S(O)₂ group), piperidine or pyrrolidine ring; optionally substituted by 1 or 2 methyl groups.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ and R⁵ are independently selected from hydrogen, methyl, carboxymethyl, —CH₂C(O)OR⁶, —CH₂C(O)NR⁶R⁷ and (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], (provided that R⁴ and R⁵ are not both hydrogen);

R⁶ and R⁷ together with the nitrogen to which they are attached form an azetidine, morpholine, piperazine, N-methylpiperazine, thiomorpholine (or derivatives thereof wherein the sulfur is oxidised to an S(O) or S(O)₂ group), piperidine or pyrrolidine ring; optionally substituted by 1 or 2 methyl groups.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

one of R⁴ and R⁵ is hydrogen or methyl, and the other is selected from carboxymethyl, —CH₂C(O)OR⁶, —CH₂C(O)NR⁶R⁷ and (2-4C)alkyl [substituted by 1 or 2 substituents independently selected from hydroxy, (1-4C)alkoxy, (1-4C)alkoxy(1-4C)alkoxy, hydroxy(2-4C)alkoxy, —C(O)OR⁶, —OC(O)R⁶, carboxy, —C(O)NR⁶R⁷, —S(O)R⁶, —S(O)₂NR⁶R⁷, —NR⁶R⁷, —NHC(O)R⁶ and —NHS(O)₂R⁶];

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

one of R⁴ and R⁵ is hydrogen or methyl, and the other is selected from —C(O)R⁶, —C(O)CH₂NR⁶R⁷, —C(O)OR⁶, —C(O)NR⁶R⁷ and —SO₂NHR⁶;

R⁶ and R⁷ together with the nitrogen to which they are attached form an azetidine, morpholine, piperazine, N-methylpiperazine, thiomorpholine (or derivatives thereof wherein the sulfur is oxidised to an S(O) or S(O)₂ group), piperidine or pyrrolidine ring; optionally substituted by 1 or 2 methyl groups.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ and R⁵ together with the nitrogen to which they are attached form a 5 or 6 membered, saturated or partially unsaturated heterocyclyl ring and optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl groups;

Particular compounds of the present invention include each individual compound described in the Examples, each of which provides a further independent aspect of the invention. In another aspect of the invention, is provided any two or more of the Examples.

### Process Section:

In a further aspect the present invention provides a process for preparing a compound of invention or a pharmaceutically-acceptable salt or an in-vivo hydrolysable ester thereof. It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon. Resins may also be used as a protecting group.

The protecting groups may be removed at any convenient stage in the synthesis using conventional techniques well known in the chemical art.

A compound of the invention, or a pharmaceutically-acceptable salt or an in vivo hydrolysable ester thereof, may be prepared by any process known to be applicable to the preparation of chemically-related compounds. Such processes, when used to prepare a compound of the invention, or a pharmaceutically-acceptable salt or an in vivo hydrolysable ester thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March or Houben-Weyl, Methoden der Organischen Chemie). The preparation of such starting materials is described within the accompanying non-limiting Examples. Alternatively, necessary starting materials are obtainable by analogous procedures to those illustrated which are within the ordinary skill of an organic chemist. Information on the preparation of necessary starting materials or related compounds (which may be adapted to form necessary starting materials) may also be found in the certain patent application publications, the contents of the relevant process sections of which are hereby incorporated herein by reference; for example WO 94/13649; WO 98/54161; WO 99/64416; WO 99/64417; WO 00/21960; WO 01/40222; WO 01/94342; WO 03/022824, JP2003335762 and WO 03/006440.

In particular we refer to our PCT patent applications WO 99/64417 and WO 00/21960 wherein detailed guidance is given on convenient methods for preparing oxazolidinone compounds.

The skilled organic chemist will be able to use and adapt the information contained and referenced within the above references, and accompanying Examples therein and also the Examples herein, to obtain necessary starting materials, and products. The terms ‘displaceable group’, ‘leaving group’ and ‘replaceable group’ are used interchangeably herein and will be understood to have the meaning conventional in the art. Examples of such groups are well known in the art and suitable examples are given below.

In a further aspect, the present invention therefore also provides that the compounds of the invention and pharmaceutically-acceptable salts and in vivo hydrolysable esters thereof, can be prepared by a process (a) to (j); and thereafter if necessary:

i) removing any protecting groups;
ii) forming a pro-drug (for example an in-vivo hydrolysable ester); and/or
iii) forming a pharmaceutically-acceptable salt;
wherein said processes (a) to (j) are as follows (wherein the variables are as defined above unless otherwise stated):
a) by modifying a substituent in, or introducing a substituent into another compound of the invention by using standard chemistry (see for example, Comprehensive Organic Functional Group Transformations (Pergamon), Katritzky, Meth-Cohn & Rees); for example: an acylamino group or thioacylamino group may be converted into another acylamino group or thioacylamino group; into a heterocyclylamino group (optionally substituted or protected on the amino-nitrogen atom); a heterocyclyl group linked through nitrogen (optionally substituted on a carbon other than a carbon adjacent to the linking nitrogen atom), for instance an optionally 4-substituted 1,2,3-triazol-1-yl group; such conversions of the acylamino group taking place either directly or through the intermediacy of one or more derivatives such as an amino group;
an 1,2,3-triazol-1-yl group may be converted by introduction of a new ring substituent or by refunctionalisation of an existing ring substituent, for instance by modifying the 4-substituent of a 4-substituted 1,2,3-triazol-1-yl group, or introducing a 4-substituent into an unsubstituted 1,2,3-triazol-1-yl group;
an amino group may be converted into a substituted amino group, for example by:

alkylation (for example with an alkyl halide, or other activated agent such as a sulfonate ester),

reductive alkylation (for example by treating with a carbonyl compound such as an aldehyde and a reducing agent such as sodium triacetoxy borohydride),

acylation (for example with an activated carboxylic acid derivative such as an acyl chloride or active ester to give an amide, an isocyanate derivative to give a urea, or a chloroformate derivative to give a carbamate, alternatively an amine may be converted into an isocyanate for example by first converting to a formamide derivative, then treating with a dehydrating agent, the resulting isocyanate derivative may then be treated with an amine or alcohol to give a urea or carbamate derivative respectively), or

sulfonylation (for example by treatment with an activated sulfonic acid derivative such as a sulfonyl chloride to give a sulphonamide);

an alcohol group may be converted into an amino group by first converting into a leaving group such as a halide, or sulfonate ester such as a para toluenesulfonate and then further conversion to an amine precursor such as an azide or phthalimide, Mitsunobu-type conditions (for example triphenylphosphine, diethylazodicarboxylate, and hydrazoic acid) may alternatively be used for this type of transformation, the amine precursor may then be converted into an amine for example by reduction of the azide (for example with aqueous triphenylphosphine) or hydrolysis of the phthalimide (for example by treating with hydrazine);

b) by reaction of one part of a compound of formula (II) (wherein X is a leaving group useful in palladium [0]coupling, for example chloride, bromide, iodide, trifluoromethylsulfonyloxy, trimethylstannyl, trialkoxysilyl, or a boronic acid residue) with one part of a compound IIa, again with a leaving group X, (wherein Y is an amine or amine derivative NR⁴R⁵ as defined hereinbefore or hereinafter, a synthetic precursor thereof, or a protected derivative (PG=protecting group) thereof) such that the pyridyl-phenyl bond replaces the phenyl-X and pyridyl-X bonds; such methods are now well known, see for instance S. P. Stanforth, Catalytic Cross-Coupling Reactions in Biaryl Synthesis, Tetrahedron, 54, 1998, 263-303; J. K. Stille, Angew Chem. Int. Ed. Eng., 1986, 25, 509-524; N. Miyaura and A Suzuki, Chem. Rev., 1995, 95, 2457-2483; D. Baranano, G. Mann, and J. F. Hartwig, Current Org. Chem., 1997, 1, 287-305; S. P. Stanforth, Tetrahedron, 54 1998, 263-303; P. R. Parry, C. Wang, A. S. Batsanov, M. R. Bryce; and B. Tarbit, J. Org. Chem., 2002, 67, 7541-7543;

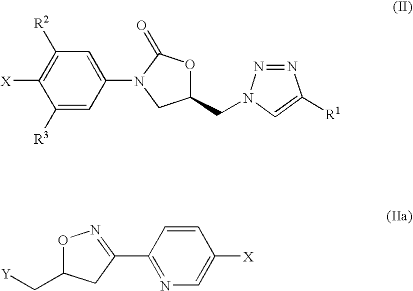

the leaving group X may be the same or different in the two molecules (II) and (IIa), as illustrated, for example, in the following scheme:

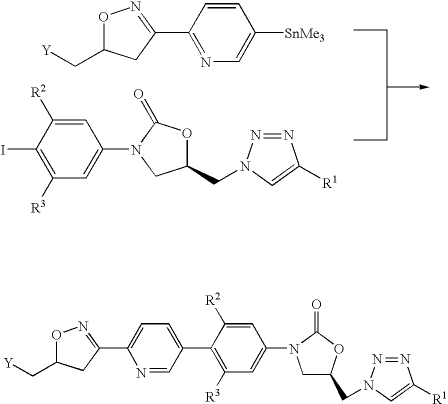

c) by reaction of a pyridyl-phenyl carbamate derivative (III) with an appropriately substituted oxirane to form an oxazolidinone ring;

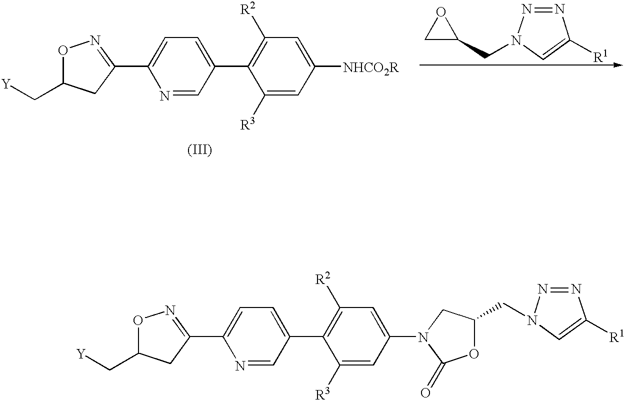

variations on this process in which the carbamate is replaced by an isocyanate or by an amine or/and in which the oxirane is replaced by an equivalent reagent X—CH₂CH(O-optionally protected)CH₂triazoleR¹ where X is a displaceable group are also well known in the art, and wherein Y is as hereinbefore defined, for example,

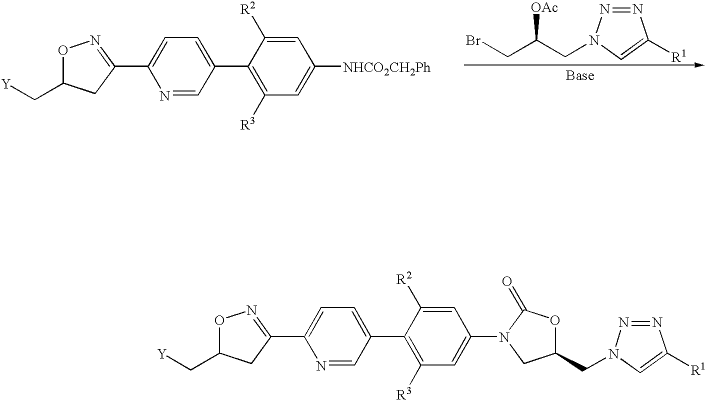

(d) by reaction of a compound of formula (IV):

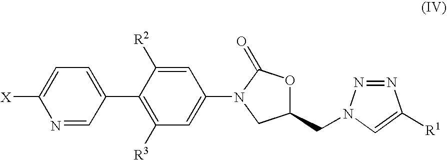

where X is a replaceable substituent—such as chloride, bromide, iodide, trifluoromethylsulfonyloxy, trimethylstannyl, trialkoxysilyl, or a boronic acid residue with a compound of the formula (V):

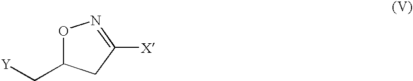

wherein X′ is a replaceable substituent (such as chloride, bromide, iodide, trifluoromethylsulfonyloxy, trimethylstannyl, trialkoxysilyl, or a boronic acid residue) and wherein Y is as hereinbefore defined; wherein the substituents X and X′ are chosen to be complementary pairs of substituents known in the art to be suitable as complementary substrates for coupling reactions catalysed by transition metals such as palladium(0);
e) by reaction of a 3-pyridylphenylbiaryl aldehyde derivative (VI) to form an isoxazoline ring at the undeveloped heteroaryl position (wherein Y is as hereinbefore defined):

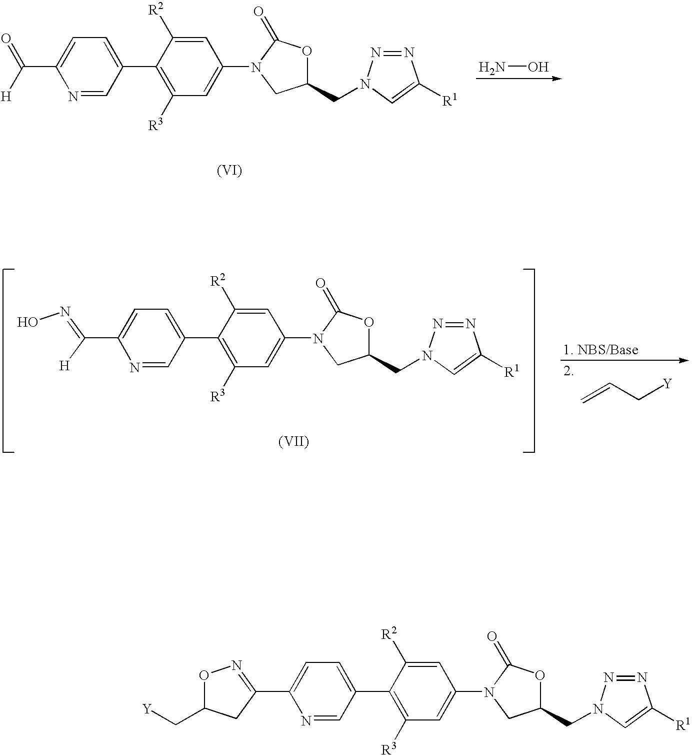

variations on this process in which the reactive intermediate (a nitrile oxide VII′) is obtained other than by oxidation of an oxime (VII) are well known in the art;

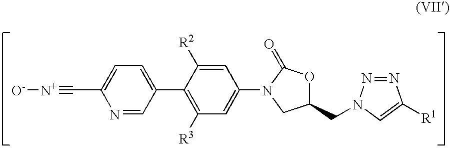

f) by formation of the triazole ring from a suitably functionalised intermediate in which the isoxazole-pyridyl-phenyl ring system is already formed, for example as illustrated by the scheme (wherein Y is as hereinbefore defined):

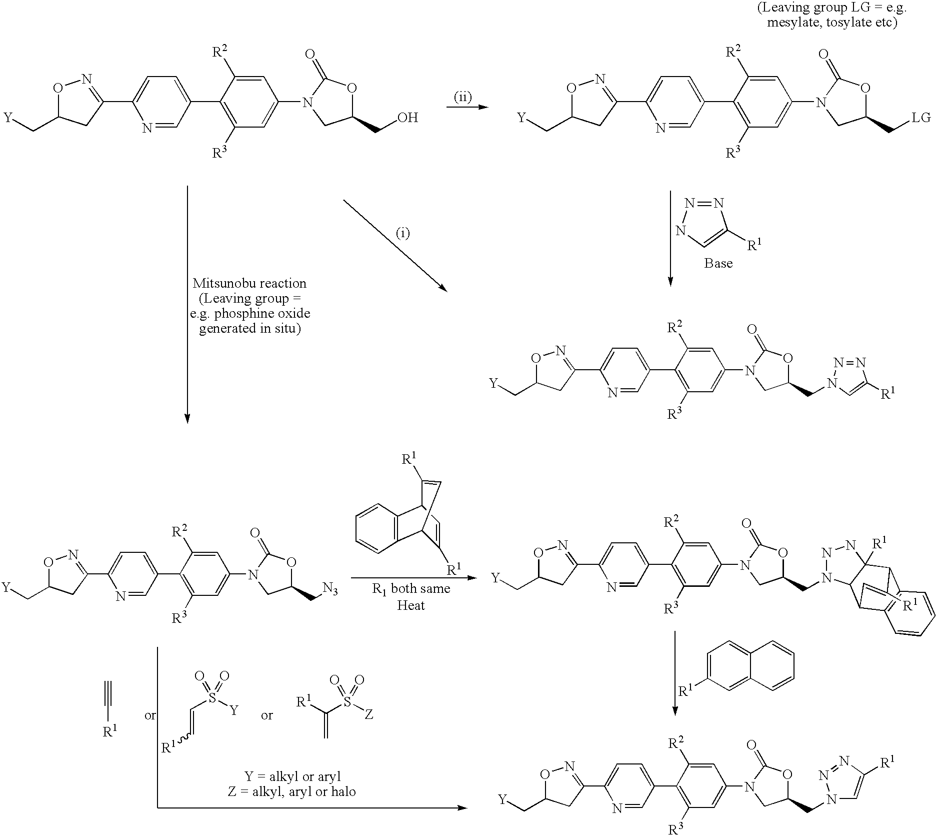

g) by cycloaddition via the azide to acetylenes, for example by reacting azidomethyl oxazolidinones with terminal alkynes using Cu(I) catalysis in e.g. aqueous alcoholic solution at ambient temperatures to give 4-substituted 1,2,3-triazoles (V. V. Rostovtsev, L. G. Green, V. V. Fokin, and K. B. Sharpless, Angew. Chem. Int. Ed., 2002, 41, 2596-2599), as illustrated below (wherein Y is as hereinbefore defined):

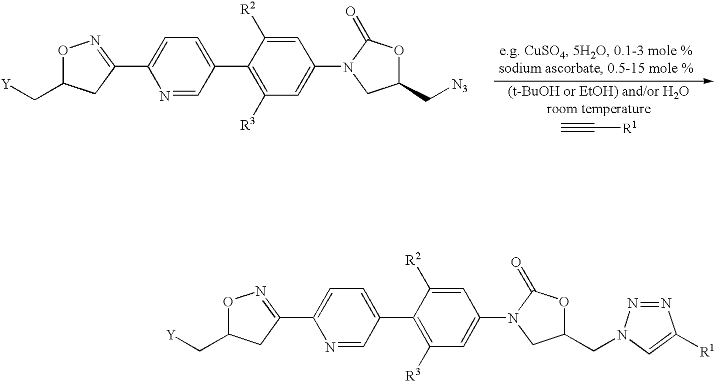

h) by reacting aminomethyloxazolidinones with 1,1-dihaloketone sulfonylhydrazones (Sakai, Kunihazu; Hida, Nobuko; Kondo, Kiyosi; Bull. Chem. Soc. Jpn., 59, 1986, 179-183; Sakai, Kunikazu; Tsunemoto, Daiei; Kobori, Takeo; Kondo, Kiyoshi; Hido, Noboko EP 103840 A2 19840328), as illustrated below (wherein Y is as hereinbefore defined);

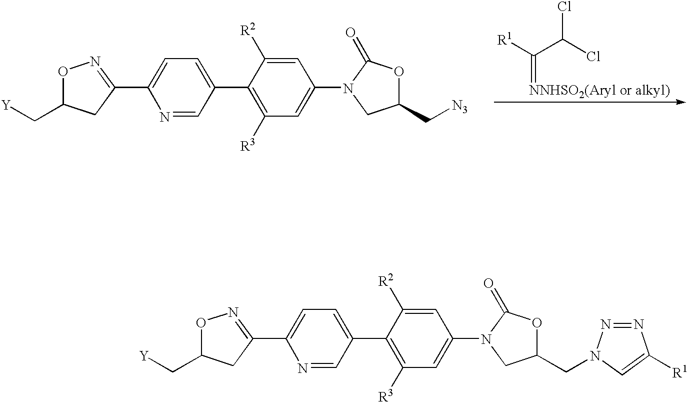

i) by reacting azidomethyl oxazolidinones with halovinylsulfonyl chlorides at a temperature between 0° C. and 100° C., either without solvent or in an inert diluent such as chlorobenzene, chloroform or dioxan, as illustrated below (wherein Y is as hereinbefore defined);

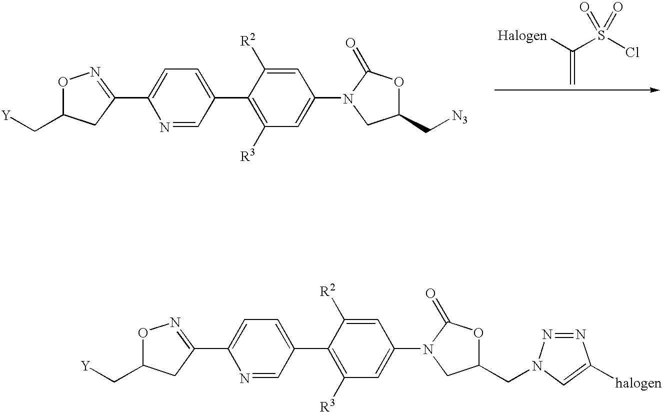

for the case when the halogen in the vinylsulfonylchloride reagent shown above is bromine see C. S. Rondestvedt, Jr. and P. K. Chang, J. Amer. Chem. Soc., 77, 1955, 6532-6540; preparation of 1-bromo-1-ethenesulfonyl chloride by C. S. Rondestvedt, Jr., J. Amer. Chem. Soc., 76, 1954, 1926-1929);
the cycloaddition reaction with 1-chloro-1-ethenesulfonyl chloride with an azide derivative in a process to form a compound of the formula (I) wherein R₁ is a 4-chloro substituent is carried out at 0° C. and 100° C., preferably at room temperature, either in an inert solvent, preferably chlorobenzene, chloroform, or dioxan, or more preferably without a solvent.
j) an alternative route to a preferred single epimer on the isoxazoline ring is via enantioselective esterase hydrolysis of a racemic mixture of esters at that pro-chiral centre to give a hydroxyl group which can be converted into a NR⁴R⁵ substituent as described herein, wherein the unwanted isomer may be recycled, for example as shown in the scheme below:

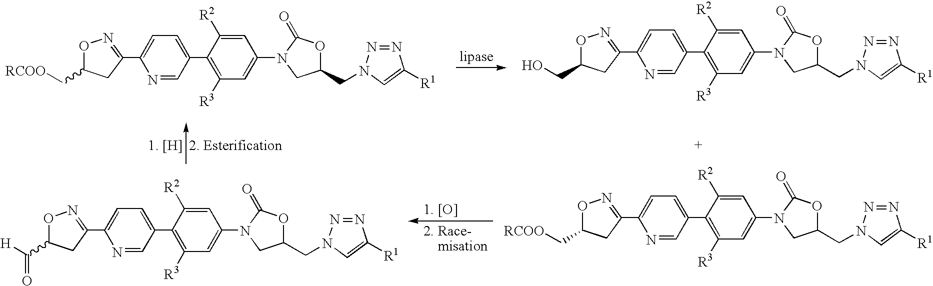

The formation of compounds of formulae (II) and (IIa) as used in b) above:

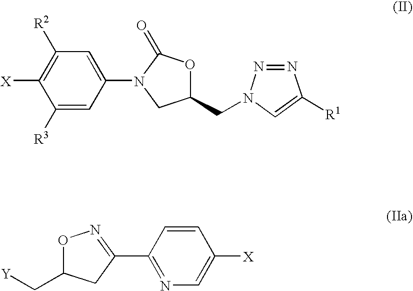

wherein each X is independently a leaving group useful in palladium [0]coupling, for example chloride, bromide, iodide, trifluoromethylsulfonyloxy, trimethylstannyl, trialkoxysilyl, or a boronic acid residue may be carried out by any method known in the art for assembling such types of compounds, see for example WO 03/022824.

For example, the 3 ring system of a compound of formula (II) may be assembled in a number of different ways as illustrated below for the unsubstituted triazole. Similar processes may be used for substituted triazoles. It will be appreciated that X in formula (II) as shown in the scheme below may be the same throughout the assembly of the 3 ring system, or may be altered at an appropriate point prior to coupling with the compound of formula (IIa); for example a compound of formula (II) wherein X is I or Br may be converted to a compound where X is a boronic acid or ester, or a trimethylstannyl derivative and then coupled with a compound of formula (Ia) with a suitable substituent X, for example Br or L. Alternatively, a compound of the formula (IIa) wherein X is a boronic acid or ester, or a trimethylstannyl derivative, may be reacted with a compound of formula (II) wherein X is a suitable halo derivative such as I or Br.

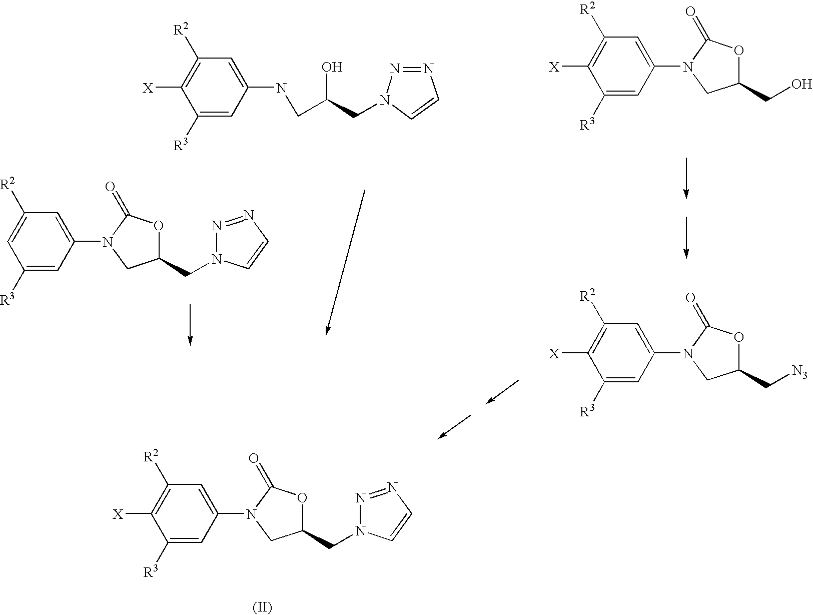

Compounds of formula (IIa) may be derived from an oxime substituted pyridine derivative as shown below, wherein X is Br or I. The oxime derivative itself may be derived from simple halo-pyridine derivatives via aldehydro-halopyridines. Where a single enantiomer is required, the chiral centre on the isoxazole ring may be introduced by any means known in the art, for example by resolution of an ester group, for instance using an enzyme such as a lipase to achieve selectivity. This process is illustrated below for a butyl ester, however it will be appreciated that other alkyl or alkenyl esters may be used, and that resolution and hydrolysis may be achieved in a single step by enzyme catalysed selective ester hydrolysis. It will also be appreciated that resolution could be achieved by enzyme catalysed esterification of a hydroxy group, followed by hydrolysis to give the chiral alcohol shown below. The hydroxy group can then be elaborated to give the required compound of formula (IIa). It will be appreciated that X in formula (IIa) as shown in the scheme below may be the same throughout the assembly of the two ring system, or may be altered at an appropriate point prior to coupling with the compound of formula (II):

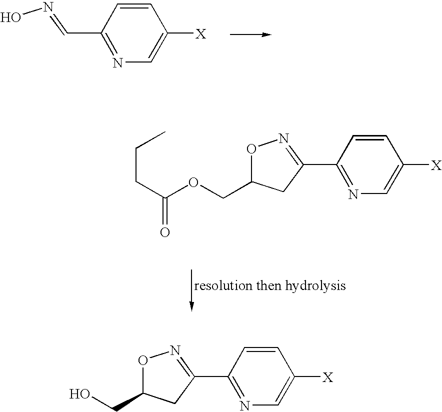

The above mentioned cyclization may alternatively be carried out with an allylic amine derivative to directly give an amine derivative as in the sequence below. Additionally, it is well known that racemic mixtures of amines can be resolved by salt formation with a chiral acid such as camphorsulfonic acid followed by crystallization. The isomers can alternatively be resolved utilizing chiral phase chromatography.

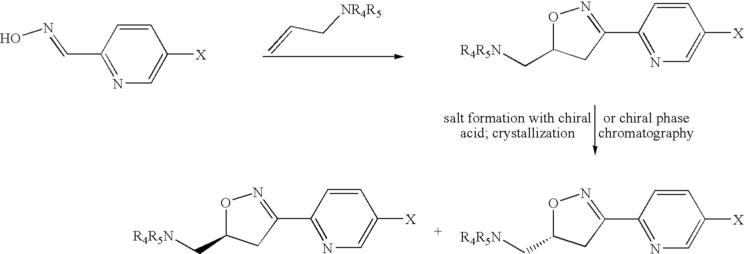

Conversion of the hydroxy group in the intermediates shown above into the NR⁴R⁵ substituent may be carried out as shown in the sequence below. Alternative precursors to the NR⁴R⁵ substituent are for example the azido, phthalimido, halo [or other leaving group KEG) such as a mesylate or tosylate ester].

It will be appreciated that the synthetic sequences shown in the scheme below may be applied at any appropriate stage during the assembly of the compound and thus that G in the schemes below may represent suitably substituted pyridyl, pyridyl-phenyl, pyridyl-phenyl-oxazolidinone or pyridyl-phenyl-oxazolidinone-methyltriazole ring systems;

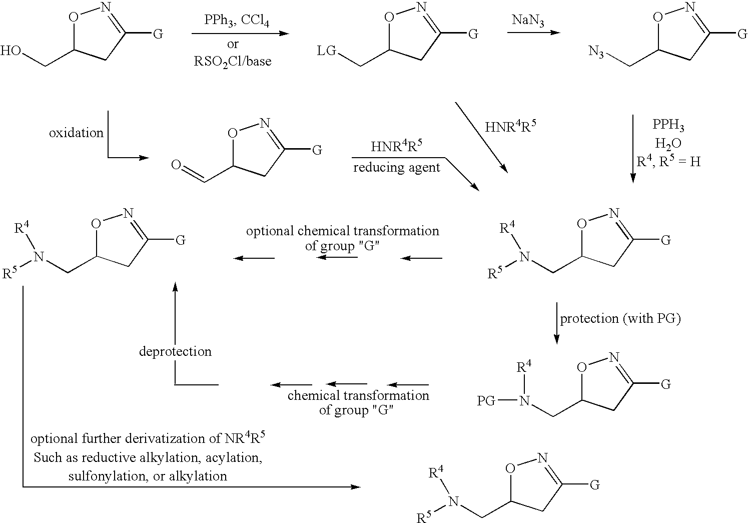

Compounds where NR⁴R⁵ together form a ring can alternatively be assembled as outlined in the sequence below. The cyclization step can alternatively be carried out using an intramolecular reductive alkylation if LG=aldehyde.

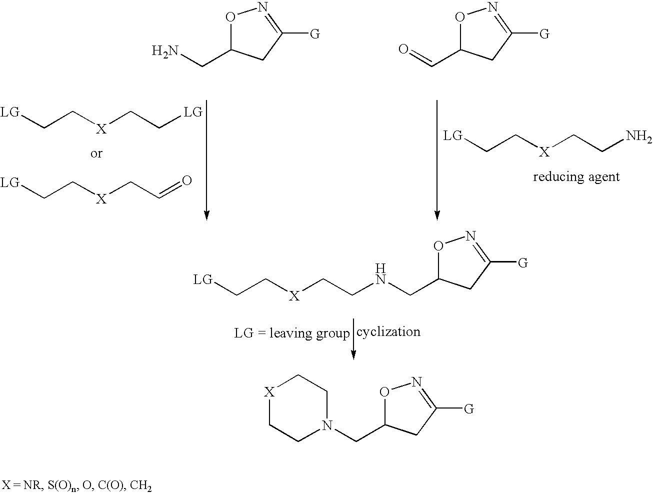

Compounds of the formula (IIa) wherein X is a boronic acid or ester and Y is NR⁴R⁵ are novel and form an independent aspect of the invention. Particular compounds of this aspect of the invention are compounds of the formula (IIa) wherein R⁴ and R⁵ are as defined in any of the aspects or embodiments of the invention described hereinbefore or hereinafter. Compounds of the formula (Ia) where in X is a halogen and Y is OR⁴ are novel and form an independent aspect of the invention. Particular compounds of this aspect of the invention are Intermediates 13, 14, 15, 16, 17, 18, 19, 20, 23, 24, 25, 26 and 31.

It will be understood that by “X is a boronic acid or ester” means X is the group —B(OR^{A})(OR^{B}), wherein R^{A} and R^{B} are independently selected from hydrogen and a (1-4C)alkyl group (such as methyl, ethyl and isopropyl), or R^{A} and R^{B}-together form a 2 or 3 carbon bridge between the two oxygen atoms attached to the boron atom to form a 5- or 6-membered ring respectively (wherein the 2 or 3 carbon bridge is optionally substituted by 1 to 4 methyl groups, for example to form a 1,1,2,2-tetramethylethylene bridge), or R^{A} and R^{B} together form a 1,2-phenyl group (thereby giving a catechol ester).

The removal of any protecting groups, the formation of a pharmaceutically-acceptable salt and/or the formation of an in-vivo hydrolysable ester or amide are within the skill of an ordinary organic chemist using standard techniques. Furthermore, details on the these steps, for example the preparation of in-vivo hydrolysable ester prodrugs has been provided, for example, in the section above on such esters.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

Compounds of the formula (I) wherein X═Br (formula (IIc) may be made from compounds of the formula (II) wherein X═H (formula (IIb) by direct bromination of a solution of the compound of formula (IIb) using bromine generated in situ from a bromate, a bromide and an acid (wherein R² and R³ are independently H or F and Rp is selected from hydrogen, halogen, cyano, methyl, cyanomethyl, fluoromethyl, difluoromethyl, trifluoromethyl and —Si[(1-4C)alkyl]₃).

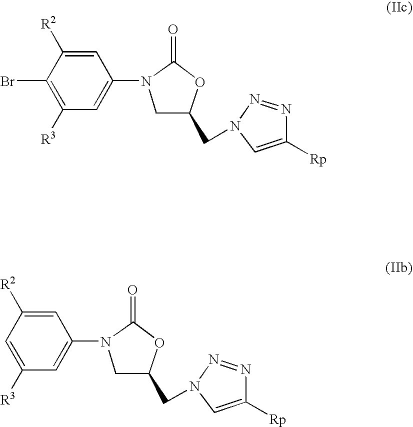

It will be appreciated that producing bromine in the reaction medium, for example by the reaction between a bromate, a bromide and acid, according to the reaction:

BrO₃—+6H⁺+5Br—→3Br₂+3H₂O

is a convenient way to circumvent problems associated with degradation of bromine solutions with time.

Conveniently, the acid and bromide may be provided together by use of hydrobromic acid. Suitably the bromide is added as a solution in water, for example an aqueous solution of hydrobromic acid, such as a 48% w/w aqueous hydrobromic acid solution. Any convenient concentration of such a solution may be used.

Conveniently the bromate is an alkali metal bromate, such as potassium bromate or sodium bromate. Suitably the bromate is added as a solution in water.

The compound of formula (IIb) may be dissolved in any suitable organic solvent. In this context, suitable means that the organic solvent must be miscible with water and must not react with the other reagents.

A suitable solvent is acetic acid. The compound of formula (IIb) may be dissolved in a mixture of said suitable organic solvent, such as acetic acid, and water.

Conveniently, the aqueous solution of bromide is added to the solution of the compound of formula (IIb), then the solution of bromate is added.

The reaction between bromate and bromide in the presence of acid is exothermic. Conveniently, a vessel containing the reaction mixture may be cooled, for instance in an ice-bath, but maintenance at a particular temperature is not essential for the yield or quality of the product produced. Conveniently a vessel containing the reaction mixture is cooled in an ice-bath such that the temperature of the reaction ranges between 10 and 30° C. during the addition of bromate.

Suitably slight molar excesses of bromate and bromide are used in comparison to the quantity of the compound of formula (IIb) used.

The rate of addition of the bromate solution is not critical. Conveniently, it is added at a rate such that the temperature of the reaction is maintained between 10 and 30° C. during the addition of bromate.

The reaction mixture may be stirred, for example at about ambient temperature, until the reaction is complete. Typically, the reaction may take 3-4 hours to complete, including the time required for addition of bromate.

After the reaction is complete, it is desirable to remove any excess bromine generated before isolation of the product. Conveniently this may be achieved by addition of a solution of metabisulfite, for example a solution of sodium metabisulfite in water. Sufficient metabisulfite is added to react with any residual bromine.

The product may be isolated by any convenient means, for example by filtration from the reaction mixture, or by dissolution into another organic solvent and appropriate washing and evaporation. If the product solidifies from the reaction mixture, it may be convenient to re-dissolve it (for example by heating the solution, for example to about 80-85° C.) and allow crystallisation in a controlled manner.

According to a further feature of the invention there is provided a compound of the invention, or a pharmaceutically-acceptable salt, or in-vivo hydrolysable ester thereof for use in a method of treatment of the human or animal body by therapy.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt, or in-vivo hydrolysable ester thereof.

The invention also provides a compound of the invention, or a pharmaceutically-acceptable salt, or in-vivo hydrolysable ester thereof, for use as a medicament; and the use of a compound of the invention of the present invention, or a pharmaceutically-acceptable salt, or in-vivo hydrolysable ester thereof, in the manufacture of a medicament for use in the production of an antibacterial effect in a warm blooded animal, such as man.

In order to use a compound of the invention, an in-vivo hydrolysable ester or a pharmaceutically-acceptable salt thereof, including a pharmaceutically-acceptable salt of an in-vivo hydrolysable ester, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the invention, an in-vivo hydrolysable ester or a pharmaceutically-acceptable salt thereof, including a pharmaceutically-acceptable salt of an in-vivo hydrolysable ester, and a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration as eye-drops, for administration by inhalation (for example as a finely divided powder or a liquid aerosol), for administration by insufflation (for example as a finely divided powder) or for parenteral administration (for example as a sterile aqueous or oily solution for intravenous, subcutaneous, sub-lingual, intramuscular or intramuscular dosing or as a suppository for rectal dosing).

In addition to the compounds of the present invention, the pharmaceutical composition of this invention may also contain (ie through co-formulation) or be co-administered (simultaneously, sequentially or separately) with one or more known drugs selected from other clinically useful antibacterial agents (for example, β-lactams, macrolides, quinolones or aminoglycosides) and/or other anti-infective agents (for example, an antifungal triazole or amphotericin). These may include carbapenems, for example meropenem or imipenem, to broaden the therapeutic effectiveness. Compounds of this invention may also be co-formulated or co-administered with bactericidal/permeability-increasing protein (BPI) products or efflux pump inhibitors to improve activity against gram negative bacteria and bacteria resistant to antimicrobial agents. Compounds of this invention may also be co-formulated or co-administered with a vitamin, for example Vitamin B, such as Vitamin B2, Vitamin B6, Vitamin B12 and folic acid. Compounds of the invention may also be formulated or co-administered with cyclooxygenase (COX) inhibitors, particularly COX-2 inhibitors.

In one aspect of the invention, a compound of the invention is co-formulated with an antibacterial agent which is active against gram-positive bacteria.

In another aspect of the invention, a compound of the invention is co-formulated with an antibacterial agent which is active against gram-negative bacteria.

In another aspect of the invention, a compound of the invention is co-administered with an antibacterial agent which is active against gram-positive bacteria.

In another aspect of the invention, a compound of the invention is co-administered with an antibacterial agent which is active against gram-negative bacteria.

The compositions of the invention may be obtained by conventional procedures using conventional pharmaceutical excipients, well known in the art. Thus, compositions intended for oral use may contain, for example, one or more colouring, sweetening, flavouring and/or preservative agents. A pharmaceutical composition to be dosed intravenously may contain advantageously (for example to enhance stability) a suitable bactericide, antioxidant or reducing agent, or a suitable sequestering agent.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol. Solubility enhancing agents, for example cyclodextrins may be used.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 50 mg to 5 g of active agent compounded with an appropriate and convenient amount of excipients which may vary from about 5 to about 98 percent by weight of the total composition. Dosage unit forms will generally contain about 200 mg to about 2 g of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

A suitable pharmaceutical composition of this invention is one suitable for oral administration in unit dosage form, for example a tablet or capsule which contains between 1 mg and 1 g of a compound of this invention, preferably between 100 mg and 1 g of a compound. Especially preferred is a tablet or capsule which contains between 50 mg and 800 mg of a compound of this invention, particularly in the range 100 mg to 500 mg.

In another aspect a pharmaceutical composition of the invention is one suitable for intravenous, subcutaneous or intramuscular injection, for example an injection which contains between 0.1% w/v and 50% w/v (between 1 mg/ml and 500 mg/ml) of a compound of this invention.

Each patient may receive, for example, a daily intravenous, subcutaneous or intramuscular dose of 0.5 mgkg⁻¹ to 20 mgkg⁻¹ of a compound of this invention, the composition being administered 1 to 4 times per day. In another embodiment a daily dose of 5 mgkg⁻¹ to 20 mgkg⁻¹ of a compound of this invention is administered. The intravenous, subcutaneous and intramuscular dose may be given by means of a bolus injection. Alternatively the intravenous dose may be given by continuous infusion over a period of time. Alternatively each patient may receive a daily oral dose which may be approximately equivalent to the daily parenteral dose, the composition being administered 1 to 4 times per day.

In the above other, pharmaceutical composition, process, method, use and medicament manufacture features, the alternative and preferred embodiments of the compounds of the invention described herein also apply.

### Antibacterial Activity:

The pharmaceutically-acceptable compounds of the present invention are useful antibacterial agents having a good spectrum of activity in vitro against standard Gram-positive organisms, which are used to screen for activity against pathogenic bacteria. Notably, the pharmaceutically-acceptable compounds of the present invention show activity against enterococci, pneumococci and methicillin resistant strains of S. aureus and coagulase negative staphylococci, together with haemophilus and moraxella strains. The antibacterial spectrum and potency of a particular compound may be determined in a standard test system.

The (antibacterial) properties of the compounds of the invention may also be demonstrated and assessed in-vivo in conventional tests, for example by oral and/or intravenous dosing of a compound to a warm-blooded mammal using standard techniques.

The following results were obtained on a standard in-vitro test system. The activity is described in terms of the minimum inhibitory concentration (MIC) determined by the agar-dilution technique with an inoculum size of 10⁴ CFU/spot. Typically, compounds are active in the range 0.01 to 256 μg/ml.

Staphylococci were tested on agar, using an inoculum of 10⁴ CFU/spot and an incubation temperature of 37° C. for 24 hours—standard test conditions for the expression of methicillin resistance.

Streptococci and enterococci were tested on agar supplemented with 5% defibrinated horse blood, an inoculum of 10⁴ CFU/spot and an incubation temperature of 37° C. in an atmosphere of 5% carbon dioxide for 48 hours—blood is required for the growth of some of the test organisms. Fastidious Gram negative organisms were tested in Mueller-Hinton broth, supplemented with hemin and NAD, grown aerobically for 24 hours at 37° C., and with an innoculum of 5×10⁴ CFU/well.

For example, the following results were obtained for the compound of Example 3

| Organism | | MIC (μg/ml) |
|---|---|---|
| Staphylococcus aureus: | MSQS | 0.5 |
| | MRQR | 0.5 |

| | |
|---|---|
| Streptococcus pneumoniae | 0.13 |
| Haemophilus influenzae | 8 |
| Moraxella catarrhalis | 1 |
| Linezolid Resistant Streptococcus pneumoniae | 1 |

MSQS = methicillin sensitive and quinolone sensitive
MRQR = methicillin resistant and quinolone resistant

### MSQS=methicillin sensitive and quinolone sensitive

### MRQR=methicillin resistant and quinolone resistant

The activity of the compounds of the invention against MAO-A was tested using a standard in-vitro assay based on human liver enzyme expressed in yeast as described in Biochem. Biophys. Res. Commun. 1991, 181, 1084-1088. Compounds of the Examples showed Ki values of ≧5 μM when measured in such an assay as above. Example 3 showed a Ki value of >22 μM. By contrast, Reference Example 16 showed a Ki value of 0.35 μM.

It will be appreciated that, as described in our patent application WO 03/072575, compounds with 4-alkyl triazoles generally demonstrate lower MAO-A inhibition than the analogous unsubstituted triazole compounds.

Certain intermediates and/or Reference Examples described hereinafter are within the scope of the invention and/or may also possess useful activity, and are provided as a further feature of the invention. A particular example is Reference Example 16.

The invention is now illustrated but not limited by the following Examples in which unless otherwise stated:—

(i) evaporations were carried out by rotary evaporation in-vacuo and work-up procedures were carried out after removal of residual solids by filtration;
(ii) operations were carried out at ambient temperature, that is typically in the range 18-26° C. and without exclusion of air unless otherwise stated, or unless the skilled person would otherwise work under an inert atmosphere;
(iii) column chromatography was used to purify compounds, either by the flash procedure on normal phase silica gel 60, 230-400 mesh, or by the flash procedure on reverse phase silica gel (C-18, RediSep, Isco, Inc.), or by HPLC on reverse phase silica gel (e.g.: Waters YMC-ODS AQ, C-18) using a Gilson 215 Platform, unless otherwise stated;
(iv) yields are given for illustration only and are not necessarily the maximum attainable;
(v) the structure of the end-products of the invention were generally confirmed by NMR and mass spectral techniques [proton magnetic resonance spectra were generally determined in DMSO-d₆ unless otherwise stated, using a Bruker spectrometer at 300, 400 or 500 MHz; chemical shifts are reported in parts per million downfield from tetramethysilane as an internal standard (δ scale) or relative to solvent. Peak multiplicities are shown thus: s, singlet; d, doublet; AB or dd, doublet of doublets; dt, doublet of triplets; dm, doublet of multiplets; t, triplet, m, multiplet; br, broad; mass spectroscopy was performed using a Micromass Quattro Micro mass spectrometer (for ESP) and an Agilent 1100 MSD instrument (for APCI); optical rotations were determined at 589 nm at 20° C. using a Perkin Elmer Polarimeter 341;
(vi) each intermediate was purified to the standard required for the subsequent stage and was characterised in sufficient detail to confirm that the assigned structure was correct; purity was assessed by HPLC, LC-MS, TLC, or NMR and identity was determined by mass spectroscopy and/or NMR spectroscopy as appropriate;
(vii) in which the following abbreviations may be used:—

DMP is N,N-dimethylformamide; DMA is N,N-dimethylacetamide; TLC is thin layer chromatography; HPLC is high pressure liquid chromatography; MPLC is medium pressure liquid chromatography; DMSO is dimethylsulfoxide; CDCl₃ is deuterated chloroform; MS is mass spectroscopy; ESP is electrospray; EI is electron impact; CI is chemical ionisation; APCI is atmospheric pressure chemical ionisation; EtOAc is ethyl acetate; MeOH is methanol; phosphoryl is (HO)₂—P(O)—O—; phosphiryl is (HO)₂—P—O—; Bleach is “Clorox” 6.15% sodium hypochlorite; EDAC is 1-[3-(dimethylamino)propyl]-3-ethylcarbodiimide; THF is tetrahydrofuran; TFA is trifluoroacetic acid; RT is room temperature; ether is diethyl ether; cf.=compare; HATU is O-(7-Azabenzotriazol-1-yl)-N,N,N′,N′-tetramethyluroniumhexafluoro phosphate

(viii) temperatures are quoted as ° C.
(ix) MP carbonate resin is a solid phase resin for use in acid Scavageing, available from Argonaut Technologies, chemical structure is PS—CH₂N(CH₂CH₃)₃⁺(CO₃²⁻)_{0.5}

### EXAMPLE 1

### (5R)-3-[4-(6-{(5S)-5-[(Dimethylamino)methyl]-4,5-dihydroisoxazol-3-yl}pyridin-3-yl)-3-fluorophenyl]-5-(1H-1,2,3-triazol-1-ylmethyl)-1,3-oxazolidin-2-one

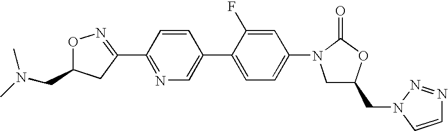

5-Bromo-2-[(5S)-5-(chloromethyl)-4,5-dihydroisoxazol-3-yl]pyridine (Intermediate 12, 0.30 g, 1.09 mmol), dimethylamine (2M solution in THF, 6 ml, 12 mmol) and tetrabutyl ammonium iodide (2 mg, catalytic amount) were combined and warmed in a sealed vial to 100° C. for 5 days. The solution was concentrated and purified by column chromatography (silica gel, 0 to 10% methanol in dichloromethane) yielding crude {[(5S)-3-(5-bromopyridin-2-yl)-4,5-dihydroisoxazol-5-yl]methyl}dimethylamine as a waxy solid (225 mg). This material (220 mg, 0.77 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 330 mg, 0.85 mmol), potassium carbonate (320 mg, 2.3 mmol), and tetrakis(triphenylphosphino)palladium(0) (90 mg, 0.078 mmol) were suspended in DMF (4 ml) and water (0.4 ml). The mixture was heated at 80° C. for 1 hour and then combined directly with a small portion silica gel. After drying the silica gel under vacuum, the material was submitted directly to column chromatography [silica gel, 1 to 20% methanol in (20% acetonitrile in dichloromethane)]. The material thus obtained was triturated with methanol: diethyl ether (1:10) followed by filtration and rinsing with diethyl ether. The title compound was thus obtained as an off-white solid (205 mg): melting point: 212° C.

MS (electrospray): 466 (M+1) for C₂₃H₂₄FN₇O₃

¹H-NMR (400 MHz. DMSO-d₆) δ: 2.50 (s, 6H); 2.89 (bm, 2H); 3.29 (dd, 1H); 3.62 (dd, 1H); 3.96 (dd, 1H); 4.30 (t, 1H); 4.86 (d, 2H); 5.03 (m, 1H); 5.18 (m, 1H); 7.42 (dd, 1H); 7.59 (dd, 1H); 7.69 (t, 1H); 7.76 (s, 1H); 8.00 (d, 1H); 8.07 (d, 1H); 8.18 (s, 1H); 8.83 (s, 1H).

### Intermediate 1: Acetic acid (SR)-3-(3-fluoro-phenyl)-2-oxo-oxazolidin-5-ylmethyl ester

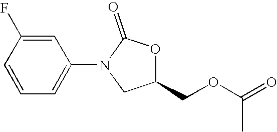

(5R)-3-(3-Fluorophenyl)-5-hydroxymethyloxazolidin-2-one (40 g, 0.189 mol, see Upjohn WO 94-13649) was suspended by stirring in dry dichloromethane (400 ml) under nitrogen. Triethylamine (21 g, 0.208 mol) and 4-dimethylaminopyridine (0.6 g, 4.9 mmol) were added, followed by dropwise addition of acetic anhydride (20.3 g, 0.199 mol) over 30 minutes, and stirring continued at ambient temperature for 18 hours. Saturated aqueous sodium bicarbonate (250 ml) was added, the organic phase separated, washed with 2% sodium dihydrogen phosphate, dried (magnesium sulfate), filtered and evaporated to give the desired product (49.6 g) as an oil.

MS (ESP): 254 (MH⁺) for C₁₂H₁₂FNO₄

NMR (300MHz) (CDCl₃) δ: 2.02 (s, 3H); 3.84 (dd, 1H); 4.16 (t, 1H); 4.25 (dd, 1H); 4.32 (dd, 1H); 4.95 (m, 1H); 6.95 (td, 1H); 7.32 (d, 1H); 7.43 (t, 1H); 7.51 (d, 1H).

### Intermediate 2: Acetic acid (SR)-3-(3-fluoro-4-iodo-phenyl)-2-oxo-oxazolidin-5-ylmethyl ester

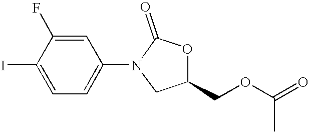

Acetic acid (5R)-3-(3-fluoro-phenyl)-2-oxo-oxazolidin-5-ylmethyl ester (Intermediate 1, 15.2 g, 60 mmol) was dissolved in a mixture of chloroform (100 ml) and acetonitrile (100 ml) under nitrogen, and silver trifluoroacetate (16.96 g, 77 mmol) were added. Iodine (18.07 g, 71 mmol) was added in portions over 30 minutes to the vigorously stirred solution, and stirring continued at ambient temperature for 18 hours. As reaction was not complete, a further portion of silver trifluoroacetate (2.64 g, 12 mmol) was added and stirring continued for 18 hours. After filtration, the mixture was added to sodium thiosulfate solution (3%, 200 ml) and dichloromethane (200 ml), and the organic phase separated, washed with sodium thiosulfate (200 ml), saturated aqueous sodium bicarbonate (200 ml), brine (200 ml), dried (magnesium sulfate), filtered and evaporated. The crude product was suspended in isohexane (100 ml), and sufficient diethyl ether added to dissolve out the brown impurity while stirring for 1 hour. Filtration gave the desired product (24.3 g) as a cream solid.

MS (ESP): 380 W) for C₁₂H₁₁FINO₄

NMR (300 MHz) (DMSO-d₆) δ: 2.03 (s, 3H); 3.82 (dd, 1H); 4.15 (t, 1H); 4.24 (dd, 1H); 4.30 (dd, 1H); 4.94 (m, 1H); 7.19 (dd, 1H); 7.55 (dd, 1H); 7.84 (t, 1H).

### Intermediate 3: (5R)-3-(3-Fluoro-4-iodophenyl)-5-hydroxymethyloxazolidin-2-one

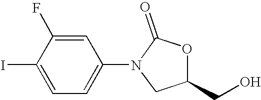

Acetic acid (5R)-3-(3-fluoro-4-iodophenyl)-2-oxo-oxazolidin-5-ylmethyl ester (Intermediate 2, 30 g, 79 mmol) was treated with potassium carbonate (16.4 g, 0.119 mmol) in a mixture of methanol (800 ml) and dichloromethane (240 ml) at ambient temperature for 25 minutes, then immediately neutralised by the addition of acetic acid (10 ml) and water (500 ml). The precipitate was filtered, washed with water, and dissolved in dichloromethane (1.2 L), the solution washed with saturated sodium bicarbonate, and dried (magnesium sulfate). Filtration and evaporation gave the desired product (23 g).

MS (ESP): 338 (MH⁺) for C₁₀H₉FINO₃

NMR (300 MHz) (DMSO-d₆) δ: 3.53 (m, 1H); 3.67 (m, 1H); 3.82 (dd, 1H); 4.07 (t, 1H); 4.70 (m, 1H); 5.20 (t, 1H); 7.21 (dd, 1H); 7.57 (dd, 1H); 7.81 (t, 1H).

### Intermediate 4: [(5R)-3-(3-Fluoro-4-iodophenyl)-2-oxo-1,3-oxazolidin-5-yl]methyl methanesulfonate

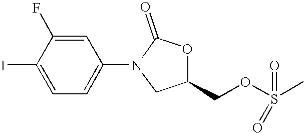

(5R)-3-(3-Fluoro-4-iodophenyl)-5-(hydroxymethyl)-1,3-oxazolidin-2-one (Intermediate 3, 25.0 g, 74.2 mmol) was stirred in dichloromethane (250 ml) at 0° C. Triethylamine (10.5 g, 104 mmol) was added followed by methanesulfonyl chloride (11.2 g, 89.0 mmol) and the reaction was stirred overnight, slowly warming to room temperature. The yellow solution was diluted with sodium bicarbonate and the compound was extracted using dichloromethane (3×250 ml). The organic layer was dried (magnesium sulfate), filtered and concentrated to give the desired product as a light yellow solid (30.3 g).

MS (ESP): 416 (MH⁺) for C₁₁H₁₁FINO₅S

¹H-NMR (300 MHz) (DMSO-d₆): 3.24 (s, 3H); 3.82 (dd, 1H); 4.17 (t, 1H); 4.43-4.52 (m, 2H); 4.99-5.03 (m, 1H); 7.21 (dd, 1H); 7.55 (dd, 1H); 7.83 (t, 1H).

### Intermediate 5: (5R)-5-(Azidomethyl)-3-(3-fluoro-4-iodophenyl)-1,3-oxazolidin-2-one

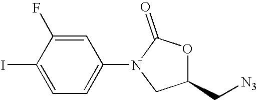

[(5R)-3-(3-Fluoro-4-iodophenyl)-2-oxo-1,3-oxazolidin-5-yl]methyl methanesulfonate (Intermediate 4, 6.14 g, 14.7 mmol) was dissolved in N,N-dimethylformamide (50 ml). Sodium azide (1.92 g, 29.6 mmol) was added and the reaction was stirred at 75° C. overnight. The yellow mixture was poured into half-saturated sodium bicarbonate and extracted using ethyl acetate. The organic layer was washed three times with water, dried (magnesium sulfate), filtered, and concentrated to give the title compound as a yellow solid (4.72 g).

MS (ESP): 363 (MH⁺) for C₁₀H₈FIN₄O₂

¹H-NMR (300 MHz) (DMSO-d₆): 3.72-3.82 (m, 3H); 4.14 (t, 1H); 4.89-4.94 (m, 1H); 7.22 (dd, 1H); 7.57 (dd, 1H); 7.83 (t, 1H).

### Intermediate 6: (SR)-3-(3-Fluoro-4-iodophenyl)-5-(1H-1,2,3-triazol-1-ylmethyl)-1,3-oxazolidin-2-one

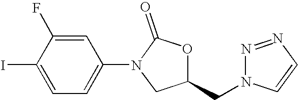

(5R)-5-(Azidomethyl)-3-(3-fluoro-4-iodophenyl)-1,3-oxazolidin-2-one (Intermediate 5, 30.3 g, 72.9 mmol) was stirred in 1,4-dioxane. Bicyclo[2.2.1]hepta-2,5-diene (40.3 g, 437 mmol) was added and the reaction was heated at 100° C. overnight. The resulting brown mixture was filtered and the desired product was obtained as a light brown solid (14.8 g).

MS (ESP): 389 (MH⁺) for C₁₂H₁₀FIN₄O₂

¹H-NMR (300 MHz) (DMSO-d₆: 3.90 (dd, 1H); 4.23 (t, 1H); 4.84 (d, 2H); 5.11-5.18 (m, 1H), 7.14 (dd, 1H); 7.49 (dd, 1H); 7.76 (s, 1H); 7.82 (t, 1H); 8.17 (s, 1H).

### Intermediate 7: (5R)-3-[3-Fluoro-4-(4,4,5,5-tetramethyl-1,3,2-dioxaborolan-2-yl)phenyl]-5-(1H-1,2,3-triazol-1-ylmethyl)-1,3-oxazolidin-2-one

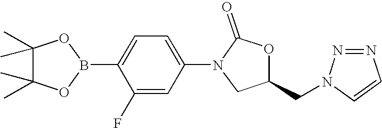

(5R)-3-(3-Fluoro-4-iodophenyl)-5-(1H-1,2,3-triazol-1-ylmethyl)-1,3-oxazolidin-2-one (Intermediate 6, 2 g, 5.15 mmol), bis(pinacolato)diboron, 2.62 g (10.3 mmol), potassium acetate, 2.5 g (25.5 mmol), and 1,1′-[bis(diphenylphosphino)ferrocene]dichloropalladium(II) dichoromethane complex, 0.38 g (0.52 mmol) were suspended in DMSO, 15 ml. The mixture was heated at 80° C. for 40 minutes to give a clear black solution. Ethyl acetate (150 ml) was then added and the mixture was filtered through celite, washed with saturated brine (2×100 ml), dried over sodium sulfate and evaporated. The dark residue was purified by chromatography (silica gel, 40 to 100% ethyl acetate in hexane, followed by 1-5% acetonitrile in ethyl acetate) to give the product as a crystalline tan solid, 1.97 g (98%). (note—highly colored impurities elute ahead of product band, extended elution required to obtain product).

NMR (300 Mz) (DMSO-d₆) δ: 1.28 (s, 12H), 3.91 (dd, 1H); 4.23 (t, 1H); 4.83 (d, 2H); 5.14 (m, 1H); 7.27 (dd, 1H); 7.37 (dd, 1H); 7.62 (t, 1H); 7.75 (s, 1H); 8.16 (s, 1H).

Alternatively:

(5R)-3-(3-Fluoro-4-iodophenyl)-5-(1H-1,2,3-triazol-1-ylmethyl)-1,3-oxazolidin-2-one (Intermediate 6, 5 g, 12.9 mmol), pinacolborane, 2.9 ml (20 mmol), triethylamine, 5.4 ml (39 mmol), and trans-dichlorobis(triphenylphosphine)palladium (II), 0.92 g (1.3 mmol) were dissolved in dioxane, 70 ml. The mixture was heated at 100° C. for 90 minutes to give a black solution, which was concentrated, dissolved in ethyl acetate, washed with brine, dried over sodium sulfate and evaporated. The residue was purified by chromatography (silica gel, 0 to 5% methanol in dichloromethane with 1% triethylamine) to give the product as a light brown solid, 3.1 g.

### Intermediate 8: 5-Bromo-N-hydroxypyridine-2-carboximidoyl chloride

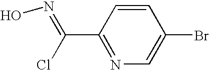

5-Bromopyridine-2-carbaldehyde oxime (49.5 g, 246.3 mmol) was dissolved in DMF (150 ml) followed by addition of N-chlorosuccinimide (39.5 g, 295.5 mmol). HCl gas was then bubbled in the solution for 20 seconds to initiate the reaction, which was then allowed to stir for 1 hr. The reaction was poured into distilled water (1 L) and the precipitate was collected by vacuum filtration. The filter cake was washed with distilled water (2×500 ml) and then dried overnight in a vacuum oven at 60° C. (−30 inches Hg) to yield the product as a white powder (55 g).

¹H-NMR (300 Mz)(CDCl₃) δ: 7.73 (d, 1H); 8.09 (d, 1H); 8.73 (s, 1H); 12.74 (s, 1H).

NOTE: Lachrymator.

### Intermediate 8a: 5-Bromopyridine-2-carbaldehyde oxime

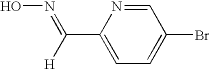

5-Bromo-pyridine-2-carbaldehyde (X. Wang et al, Tetrahedron Letters 41 (2000), 4335-4338) (60 g, 322 mmol) was added to methanol (700 ml) and then water was added (700 ml) followed by addition of hydroxylamine hydrochloride (28 g, 403 mmol). Sodium carbonate (20.5 g, 193.2 mmol) in water (200 ml) was added and the reaction was stirred for 30 minutes. Water (500 ml) was then added and the precipitate was filtered and washed with water (2×300 ml) to give the desired product (60 g).

NMR (DMSO-d₆) δ: 7.75 (d, 1H); 8.09 (t, 2H), 8.72 (s, 1H); 11.84 (s, 1H).

### Intermediate 9: [3-(5-Bromopyridin-2-yl)-4,5-dihydroisoxazol-5-yl]methyl butyrate

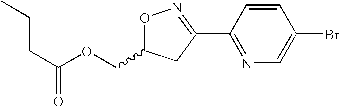

5-Bromo-N-hydroxypyridine-2-carboximidoyl chloride (Intermediate 8.46 g, 195.7 mmol) was added to EtOAc (200 ml) followed by addition of allyl butyrate (145 ml, 1020.4 mmol) and the solution was cooled to 0° C. Triethylamine (30 ml, 215.8 mmol) in EtOAc (100 ml) was then added dropwise over 1 hour. The reaction was then allowed to stir for 1 hour at 0° C. and then EtOAc (1 L) was added. The precipitate was removed by vacuum filtration and the filtrate was concentrated in vacuo to yield the product (65 g).

¹H-NMR (DMSO-d₆) δ: 0.81 (t, 3H); 1.43 (m, 2H); 2.24 (t, 2H); 3.21 (dd, 1H); 3.54 (dd, 1H); 4.13 (dd, 1H); 4.23 (dd, 1H); 5.01 (m, 1H); 7.85 (dd, 1H); 8.12 (dd, 1H); 8.81 (d, 1H).

### Intermediate 10: (5S)-3-(5-Bromopyridin-2-yl)-4,5-dihydroisoxazol-5-yl]methyl butyrate

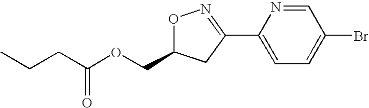

(+) Isomer assigned as (5S) based on comparison with Chem. Lett. 1993 p. 1847.

Racemic [3-(5-bromopyridin-2-yl)-4,5-dihydroisoxazol-5-yl]methyl butyrate (Intermediate 9, 80 g, 0.244 mol) was dissolved in acetone (4 L), and 0.1 M potassium phosphate buffer (pH˜7) (4 L) was added with vigorous stirring to give a clear yellow solution. PS-lipase (1.45 g, Sigma cat no L-9156) was added and the mixture was gently stirred at ambient temp. for 42 hrs. The solution was divided into 3 equal volumes of ˜2.6 L and each was extracted with dichloromethane (2×1 L), the pooled organic phases were dried over sodium sulfate and evaporated. The unreacted [(5S)-3-(5-bromopyridin-2-yl)-4,5-dihydroisoxazol-5-yl]methyl butyrate was isolated via flash column chromatography (9:1 hexane:ethyl acetate) as a clear yellow oil, 36.4 g (45.5%).

### Intermediate 11: [(5S)-3-(5-Bromopyridin-2-yl)-4,5-dihydroisoxazol-5-yl]methanol

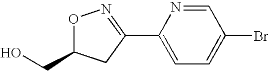

[(5S)-3-(5-Bromopyridin-2-yl)-4,5-dihydroisoxazol-5-yl]methyl butyrate (Intermediate 10, 16.88 g, 0.051 mol) was dissolved in methanol (110 ml). 50% Aqueous sodium hydroxide (3.6 ml, 0.068 mol) was added. The solution was stirred at RT for 15 minutes, 1M HCl (75 ml) was added, followed by concentration in vacuo to ˜100 ml total volume. Water (˜50 ml) was added, and the white precipitate was collected and rinsed with water. The filtrate was extracted twice with ethyl acetate, the organic layers were pooled, dried over sodium sulfate and evaporated. The solid residue was collected and rinsed with 10:1 hexane:ethyl acetate, then combined with the initial precipitate before drying in vacuo to give the title compound as a white crystalline solid, 12.3 g (93%). Chiral HPLC analysis indicated <0.5% of the (−) isomer was present. [α]_{D}=+139 (c=0.01 g/ml in methanol).

### Intermediate 12: 5-Bromo-2-[(5S)-5-(chloromethyl)-4,5-dihydroisoxazol-3-yl]pyridine

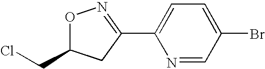

[(5S)-3-(5-Bromopyridin-2-yl)-4,5-dihydroisoxazol-5-yl]methanol (Intermediate 11, 4 g, 16 mmol) was dissolved in dichloromethane (80 ml). Triphenylphosphine (7.0 g, 26.7 mmol) and carbon tetrachloride (9 ml, 93 mmol) were added and the mixture was stirred at room temperature for 2 hours. The solution was concentrated and purified by flash chromatography (silica gel, 7:3 hexane: dichloromethane) to yield the title product as a white solid (3.9 g).

¹H-NMR (300 MHz, CDCl₃) δ: 3.42-3.73 (m, 4H); 4.98-5.08 (m, 1H); 7.84 (dd, 1H); 7.90 (d, 1H); 8.65 (d, 1H).

### EXAMPLE 2

### (5R)-3-[3-Fluoro-4-(6-{(5S)-5-[(methylamino)methyl]-4,5-dihydroisoxazol-3-yl}pyridin-3-yl)phenyl]-5-(1H-1,2,3-triazol-1-ylethyl)-1,3-oxazolidin-2-one

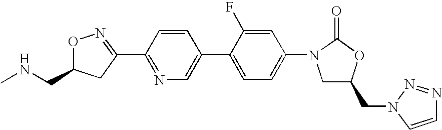

5-Bromo-2-[(5S)-5-(chloromethyl)-4,5-dihydroisoxazol-3-yl]pyridine (Intermediate 12, 0.40 g, 1.45 mmol), methyl amine (2M solution in THF, 8 ml, 16 mmol) and tetrabutyl ammonium iodide (2 mg, catalytic amount) were combined and warmed in a sealed vial to 100° C. for 5 days. The solution was concentrated and purified by column chromatography (silica gel, 0 to 10% methanol in dichloromethane) yielding crude {[(5S)-3-(5-bromopyridin-2-yl)-4,5-dihydroisoxazol-5-yl]methyl}methylamine as a waxy solid (160 mg). This material (155 mg, 0.57 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 245 mg, 0.63 mmol), potassium carbonate (240 mg, 1.74 mmol), and tetrakis(triphenylphosphino)palladium(0) (66 mg, 0.057 mmol) were suspended in DMF (3 ml) and water (0.3 ml). The mixture was heated at 80° C. for 1 hour and then combined directly with a small portion silica gel. After drying the silica gel under vacuum, the material was submitted directly to column chromatography [silica gel, 1 to 20% methanol in (20% acetonitrile in dichloromethane)]. The material thus obtained was triturated with methanol: diethyl ether (1:10) followed by filtration and rinsing with diethyl ether. The title compound was thus obtained as an off-white solid (75 mg): melting point: 215° C.

MS (electrospray): 452 (M+1) for C₂₂H₂₂FN₇O₃

¹H-NMR (400 MHz. DMSO-d₆) δ: 2.57 (s, 3H); 3.14 (d, 2H); 3.37 (dd, 1H); 3.67 (dd, 1H); 3.96 (dd, 1H); 4.30 (t, 1H); 4.86 (d, 2H); 5.05 (m, 1H); 5.19 (m, 1H); 7.42 (dd, 1H); 7.59 (dd, 1H); 7.69 (t, 1H); 7.76 (s, 1H); 8.01 (d, 1H); 8.08 (d, 1H); 8.18 (s, 1H); 8.24 (bs, 1H); 8.84 (s, 1H).

### EXAMPLE 3

### (5R)-3-(3-Fluoro-4-{6-[(5S)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one

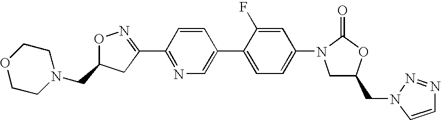

4-{[(5S)-3-(5-Bromopyridin-2-yl)-4,5-dihydroisoxazol-5-yl]methyl}morpholine (Intermediate 13, 325 mg, 0.99 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 350 mg, 0.90 mmol), potassium carbonate (420 mg, 3.04 mmol), and tetrakis(triphenylphosphino)palladium(0) (120 mg, 0.10 mmol) were suspended in DMP (5 ml) and water (0.5 ml). The mixture was heated at 80° C. for 30 minutes, allowed to cool, and filtered. The solids were rinsed with acetonitrile and the combined filtrate was evaporated, redissolved in acetonitrile and adsorbed on silica gel. The adsorbed material was purified by column chromatography [silica gel, 1 to 10% methanol in (20% acetonitrile in dichloromethane)]. The material thus obtained was stirred with hot methanol (20 ml) and diluted with diethyl ether (20 ml) followed by filtration and rinsing with diethyl ether. The title compound was thus obtained as an off-white solid (154 mg): melting point: 230° C.

MS (electrospray): 508 (M+1) for C₂₅H₂₆FN₇O₄

¹H-NMR (400 MHz DMSO-d₆) δ: 2.47 (bm, 4H); 2.56 (m, 2H); 3.27 (dd, 1H); 3.54 (dd, 1H); 3.56 (bm, 4H); 3.96 (dd, 1H); 4.29 (t, 1H); 4.86 (d, 2H); 4.94 (m, 1H); 5.19 (m, 1H); 7.42 (dd, 1H); 7.59 (dd, 1H); 7.69 (t, 1H); 7.76 (s, 1H); 7.99 (d, 1H); 8.05 (d, 1H); 8.18 (s, 1H); 8.81 (s, 1H).

### EXAMPLE 3a

### (5R)-3-(3-Fluoro-4-{6-[(5S)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one, hydrochloride salt

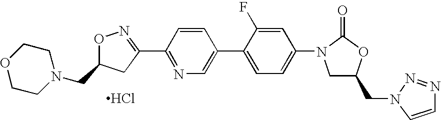

(5R)-3-(3-Fluoro-4-{6-[(5S)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one (Example 3, 1 g, 1.97 mmol), was dissolved in a mixture of acetonitrile (5 ml), methanol (5 ml), DMF (5 ml) and dichloromethane (15 ml), then cooled to 0° C. Hydrochloric acid (4 M solution in dioxane, 0.5 ml, 2 mmol) was added and the clear solution was diluted with diethyl ether (30 ml) to give a suspension. The solids were collected, rinsed with diethyl ether and dried under vacuum at 50° C. to give the title compound as an off-white solid (909 mg) melting point: 242° C.

MS (electrospray): 508 (M+1) for C₂₅H₂₆FN₇O₄

¹H-NMR (300 MHz, DMSO-d₆) δ: 3.18 (bm, 2H); 3.33-3.55 (bm, 4H); 3.72-3.88 (bm, 4H); 3.95 (m, 3H); 4.30 (t, 1H); 4.86 (d, 2H); 5.19 (m, 1H); 5.37 (bm, 1H); 7.42 (dd, 1H); 7.59 (dd, 1H); 7.69 (t, 1H); 7.77 (s, 1H); 8.02 (d, 1H); 8.09 (d, 1H); 8.19 (s, 1H); 8.85 (s, 1H); 11.17 (bs, 1H).

### EXAMPLE 3b

### (5R)-3-(3-Fluoro-4-{6-[(5S)-5-(morpholin-4-ylmethyl)-4,5-dihydroisoxazol-3-yl]pyridin-3-yl}phenyl)-5-(1H-1,2,3-triazol-1-ylethyl)-1,3-oxazolidin-2-one, hydrobromide salt

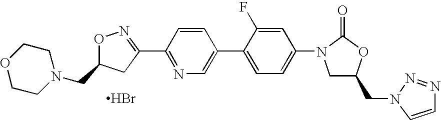

To a solution of Example 3 (380 mg, 7.5 mmol) in isopropanol/dichloromethane (1:1, 20 mL) was added under vigorous stirring HBr in acetic acid (33%, 0.3 mL). The solvent was evaporated under reduced pressure and the residue codistilled twice with a mixture of isopropanol/water (1:1, 2×5 mL). The residue was dissolved in hot isopropanol/water (2:1, 20 mL) and product precipitated by addition of cold isopropanol (20 mL). The solid was collected by filtration and dried under reduced pressure at 48° C. to give 367 mg of the hydrobromide salt of Example 3 as a colourless solid, mp>280° C. (dec.).

MS (electrospray): 508 (M+1) for C₂₅H₂₆N₇O₄

¹H-NMR (300 MHz, DMSO-d₆) δ: 3.20 (bm, 2H); 3.45-3.60 (bm, 4H); 3.70-3.84 (bm, 4H); 3.92-4.01 (m, 3H); 4.30 (t, 1H); 4.86 (d, 2H); 5.19 (m, 1H); 5.30 (bm, 1H); 7.43 (dd, 1H); 7.59 (dd, 1H); 7.70 (t, 1H); 7.77 (s, 1H); 8.03 (d, 1H); 8.10 (d, 1H); 8.19 (s, 1H); 8.85 (s, 1H); 10.13 (bs, 1H).

### EXAMPLE 3c

### (5R)-3-(3-Fluoro-4-{6-[(5S)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one, methanesulfonic acid salt

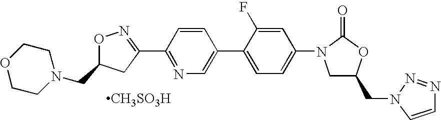

(5R)-3-(3-Fluoro-4-{6-[(5S)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one (Example 3, 100 mg, 0.197 mmol), was dissolved in 1:1 acetonitrile:methanol (50 ml) with warming. Methanesulfonic acid (0.5 M solution in methanol, 0.4 ml, 0.2 mmol) was added and the clear solution was concentrated to 10 ml and diluted with ethyl acetate to give a suspension. The solids were collected, rinsed with ethyl acetate and dried under vacuum at 50° C. to give the title compound as an off-white solid (100 mg) melting point: 165-168° C.

MS (electrospray): 508 (M+1) for C₂₅H₂₆FN₇O₄

¹H-NMR (300 MHz. DMSO-d₆) δ: 2.28 (s, 3H); 3.19 (bm, 2H); 3.40-3.59 (bm, 4H); 3.64-3.82 (bm, 4H); 3.92-4.02 (m, 3H); 4.30 (t, 1H); 4.86 (d, 2H); 5.19 (m, 1H); 5.28 (bm, 1H); 7.42 (dd, 1H); 7.59 (dd, 1H); 7.69 (t, 1H); 7.77 (s, 1H); 8.03 (d, 1H); 8.10 (d, 1H); 8.18 (s, 1H); 8.85 (s, 1H); 10.02 (bs, 1H).

### Intermediate 13: 4-{[(5S)-3-(5-Bromopyridin-2-yl)-4,5-dihydroisoxazol-5-yl]methyl}morpholine

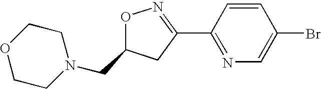

5-Bromo-2-[(5S)-5-(chloromethyl)-4,5-dihydroisoxazol-3-yl]pyridine (0.30 g, 1.09 mmol), morpholine (1 g, 11.5 mmol), tetrabutyl ammonium iodide (2 mg, catalytic amount) and DMSO (1 ml) were combined and warmed to 115° C. for 16 hours. The solution was diluted with water and extracted twice with ethyl acetate. The pooled organic layers were dried over sodium sulfate and evaporated to give crude title product as a waxy yellow solid (345 mg).

MS (electrospray): 327 (M+1) for C₁₃H₁₆BrN₃O₂

### EXAMPLE 4

### (5R)-3-[3-Fluoro-4-(6-{(5S)-5-[(4-methylpiperazin-1-yl)methyl]-4,5-dihydroisoxazol-3-yl}pyridin-3-yl)phenyl]-5-(1H-1,2,3-triazol-1-ylmethyl)-1,3-oxazolidin-2-one

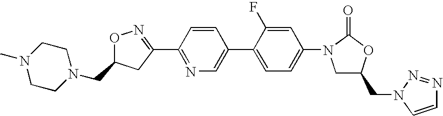

1-{[(5S)-3-(5-Bromopyridin-2-yl)-4,5-dihydroisoxazol-5-yl]methyl})-methylpiperazine Intermediate 14, 360 mg, 1.06 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 350 mg, 0.90 mmol), potassium carbonate (420 mg, 3.04 mmol), and tetrakis(triphenylphosphino)palladium(0) (120 mg, 0.10 mmol) were suspended in DMF (5 ml) and water (0.5 ml). The mixture was heated at 80° C. for 60 minutes, allowed to cool, adsorbed directly onto silica gel and dried in vacuo. The adsorbed material was purified by column chromatography [silica gel, 1 to 20% methanol in (20% acetonitrile, 1% triethylamine in dichloromethane)]. The material thus obtained was stirred with hot methanol (10 ml) and diluted with diethyl ether (60 ml) followed by filtration and rinsing with diethyl ether. The title compound was thus obtained as an off-white solid (190 mg): melting point: 218° C.

MS (electrospray): 521 (M+1) for C₂₆H₂₉FN₈O₃

¹H-NMR (400 MHz. DMSO-d₆) δ: 2.22 (bm, 4H); 2.55 (bm, 7H); 3.25 (dd, 1H); 3.29 (m, 2H); 3.52 (dd, 1H); 3.96 (dd, 1H); 4.29 (t, 1H); 4.86 (d, 2H); 4.92 (m, 1H); 5.19 (m, 1H); 7.42 (dd, 1H); 7.59 (dd, 1H); 7.69 (t, 1H); 7.76 (s, 1H); 7.99 (d, 1H); 8.05 (d, 1H); 8.18 (s, 1H); 8.81 (s, 1H).

### Intermediate 14: 1-{[(5S)-3-(5-Bromopyridin-2-yl)-4,5-dihydroisoxazol-5-yl]methyl}-4-methylpiperazine

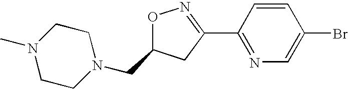

5-Bromo-2-[(5S)-5-(chloromethyl)-4,5-dihydroisoxazol-3-yl]pyridine (Intermediate 12, 0.30 g, 1.09 mmol), 1-methylpiperazine (0.9 ml, 10 mmol), tetrabutyl ammonium iodide (2 mg, catalytic amount) and DMSO (1 ml) were combined and warmed to 100° C. for 20 hours. The solution was diluted with water and extracted twice with ethyl acetate. The pooled organic layers were dried over sodium sulfate and evaporated to give crude title product as a waxy yellow solid (360 mg).

MS (electrospray): 340 (M+1) for C₁₄H₁₉BrN₄O

### EXAMPLE 5

### (5R)-3-{3-Fluoro-4-[6-((5S)-5-{[(2-hydroxyethyl)amino]methyl}-4,5-dihydroisoxazol-3-yl)pyridin-3-yl]phenyl}-5-(1H-1,2,3-triazol-1-ylmethyl)-1,3-oxazolidin-2-one

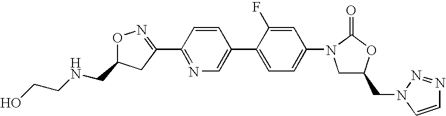

2-({[(5S)-3-(5-Bromopyridin-2-yl)-4,5-dihydroisoxazol-5-yl]methyl}amino)ethanol (Intermediate 15, 315 mg, 1.05 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 350 mg, 0.90 mmol), potassium carbonate (420 mg, 3.04 mmol), and tetrakis(triphenylphosphino)palladium(0) (120 mg, 0.10 mmol) were suspended in DMF (5 ml) and water (0.5 ml). The mixture was heated at 80° C. for 60 minutes, allowed to cool, adsorbed directly onto silica gel and dried in vacuo. The adsorbed material was purified by column chromatography [silica gel, 1 to 20% methanol in (20% acetonitrile, 1% triethylamine in dichloromethane)]. The material thus obtained was stirred with hot methanol (10 ml) and diluted with diethyl ether (60 ml) followed by filtration and rinsing with diethyl ether. The title compound was thus obtained as an off-white solid (170 mg): melting point: 190° C.

MS (electrospray): 482 (M+1) for C₂₃H₂₄FN₇O₄

¹H-NMR (400 MHz, DMSO-d₆) δ: 2.64 (t, 2H); 2.78 (d, 2H); 3.29 (dd, 1H); 3.45 (m, 2H); 3.50 (dd, 1H); 3.96 (dd, 1H); 4.29 (t, 1H); 4.53 (t, 1H); 4.85 (m, 1H); 4.86 (d, 2H); 5.18 (m, 1H); 7.42 (dd, 1H); 7.59 (dd, 1H); 7.69 (t, 1H); 7.76 (s, 1H); 7.99 (d, 1H); 8.05 (d, 1H); 8.18 (s, 1H); 8.81 (s, 1H).

### Intermediate 15: 2-({[(5S)-3-(5-Bromopyridin-2-yl)-4,5-dihydroisoxazol-5-yl]methyl}amino)ethanol

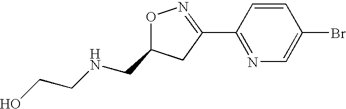

5-Bromo-2-[(5S)-5-(chloromethyl)-4,5-dihydroisoxazol-3-yl]pyridine (Intermediate 12, 0.30 g, 1.09 mmol), ethanolamine (0.6 ml, 9.7 mmol), tetrabutyl ammonium iodide (2 mg, catalytic amount) and DMSO (1 ml) were combined and warmed to 100° C. for 20 hours. The solution was diluted with water and extracted twice with ethyl acetate. The pooled organic layers were dried over sodium sulfate and evaporated to give crude title product as a waxy yellow solid (315 mg).

MS (electrospray): 301 (M+1) for C₁₁H₁₄BrN₃O₂

### EXAMPLE 6

### (5R)-3-[4-(6-{(5S)-5-[(Butylamino)methyl]-4,5-dihydroisoxazol-3-yl}pyridin-3-yl)-3-fluorophenyl]-5-(1H-1,2,3-triazol-1-ylmethyl)-1,3-oxazolidin-2-one

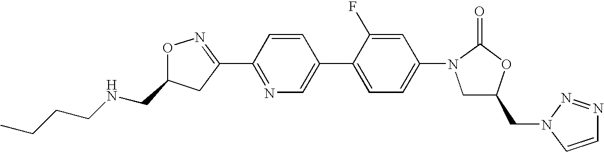

N-{[(5S)-3-(5-Bromopyridin-2-yl)-4,5-dihydroisoxazol-5-yl]methyl}butan-1-amine (Intermediate 16, 335 mg, 1.07 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 350 mg, 0.90 mmol), potassium carbonate (420 mg, 3.04 mmol), and tetrakis(triphenylphosphino)palladium(0) (120 mg, 0.10 mmol) were suspended in DMP (5 ml) and water (0.5 ml). The mixture was heated at 80° C. for 60 minutes, allowed to cool, adsorbed directly onto silica gel and dried in vacuo. The adsorbed material was purified by column chromatography [silica gel, 1 to 20% methanol in (20% acetonitrile in dichloromethane)]. The material thus obtained was stirred with hot methanol (10 ml) and diluted with diethyl ether (60 ml) followed by filtration and rinsing with diethyl ether. The title compound was thus obtained as an off-white solid (190 mg): melting point: 230° C.

MS (electrospray): 494 (M+1) for C₂₅H₂₈FN₇O₃

¹H-NMR (400 MHz. DMSO-d₆) δ: 0.88 (t, 3H); 1.31 (m, 2H); 1.52 (m, 2H); 2.81 (t, 2H); 3.06 (d, 2H); 3.35 (dd, 1H); 3.63 (dd, 1H); 3.96 (dd, 1H); 4.30 (t, 1H); 4.86 (d, 2H); 4.99 (m, 1H); 5.19 (m, 1H); 7.42 (dd, 1H); 7.59 (dd, 1H); 7.69 (t, 1H); 7.76 (s, 1H); 8.01 (d, 1H); 8.08 (d, 1H); 8.18 (s, 1H); 8.84 (s, 1H).

### Intermediate 16: N-{[(5S)-3-(5-Bromopyridin-2-yl)-4,5-dihydroisoxazol-5-yl]methyl}butan-1-anine

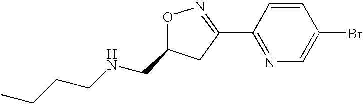

5-Bromo-2-[(5S)-5-(chloromethyl)-4,5-dihydroisoxazol-3-yl]pyridine (Intermediate 12, 0.30 g, 1.09 mmol), n-butylamine (1.0 ml, 10 mmol), tetrabutyl ammonium iodide (2 mg, catalytic amount) and DMSO (1 ml) were combined and warmed to 100° C. for 20 hours. The solution was diluted with water and extracted twice with ethyl acetate. The pooled organic layers were dried over sodium sulfate and evaporated to give crude title product as a waxy yellow solid (335 mg).

MS (electrospray): 313 (M+1) for C₁₃H₁₈BrN₃O

### EXAMPLE 7

### (5R)-3-(3-Fluoro-4-{6-[(5S)-5-(thiomorpholin-4-ylmethyl)-4,5-dihydroisoxazol-3-yl]pyridin-3-yl}phenyl)-5-(1H-1,2,3-triazol-1-ylmethyl)-1,3-oxazolidin-2-one

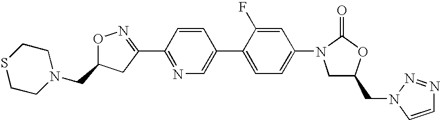

4-{[(5S)-3-(5-Bromopyridin-2-yl)-4,5-dihydroisoxazol-5-yl]methyl}thiomorpholine (Intermediate 17, 280 mg, 0.82 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 349 mg, 0.90 mmol), potassium carbonate (340 mg, 2.46 mmol), and tetrakis(triphenylphosphino)palladium(0) (95 mg, 0.082 mmol) were suspended in DMF (5 ml) and water (0.5 ml). The mixture was heated at 80° C. for 1 hour, allowed to cool, and filtered. The filtrate was combined with silica gel and dried in vacuo. The adsorbed material was purified by column chromatography (silica gel, 1 to 10% methanol: dichloromethane). The material thus obtained was dissolved in warm methanol (5 ml) and dichloromethane (5 ml), the solution was concentrated with heating to 5 ml, and allowed to cool, yielding a precipitate. The solids were filtered and rinsed with methanol, water, again with methanol, then diethyl ether and dried in vacuo. The title compound was thus obtained as an off-white solid (150 mg): melting point: 222-225° C.

MS (electrospray): 524 (M+1) for C₂₅H₂₆FN₇O₃S

¹H-NMR (400 MHz, DMSO-d₆) δ: 2.58 (bm, 6H); 2.75 (bm, 4H); 3.24 (dd, 1H); 3.52 (dd, 1H); 3.96 (dd, 1H); 4.30 (t, 1H); 4.86 (d, 2H); 4.93 (m, 1H); 5.19 (m, 1H); 7.42 (dd, 1H); 7.59 (dd, 1H); 7.69 (t, 1H); 7.76 (s, 1H); 7.99 (d, 1H); 8.05 (d, 1H); 8.18 (s, 1H); 8.82 (s, 1H).

### Intermediate 17: 4-{[(5S)-3-(5-Bromopyridin-2-yl)-4,5-dihydroisoxazol-5-yl]methyl}thiomorpholine

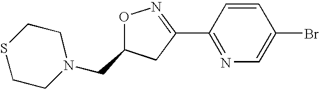

5-Bromo-2-[(5S)-5-(chloromethyl)-4,5-dihydroisoxazol-3-yl]pyridine (Intermediate 12, 1 g, 3.26 mmol), thiomorpholine (3 g, 29 mmol), tetrabutyl ammonium iodide (2 mg, catalytic amount) and DMSO (3 ml) were combined and warmed to 110° C. for 16 hours. The solution was diluted with water and extracted twice with ethyl acetate. The pooled organic layers were dried over sodium sulfate, evaporated and purified by flash chromatography (silica gel, 10 to 50% ethyl acetate in hexanes) to give the title compound as an off-white solid (635 mg).

¹H-NMR (400 MHz, DMSO-d₆) δ: 2.57 (bm, 6H); 2.72 (bm, 4H); 3.17 (dd, 1H); 3.46 (dd, 1H); 4.91 (m, 1H); 7.84 (d, 1H); 8.11 (d, 1H); 8.77 (s, 1H).

### EXAMPLE 8

### (5R)-3-[3-Fluoro-4-(6-{(5S)-5-[(1-oxidothiomorpholin-4-yl)methyl]-4,5-dihydroisoxazol-3-yl}pyridin-3-yl)phenyl]-5-(1H-1,2,3-triazol-1-ylmethyl)-1,3-oxazolidin-2-one

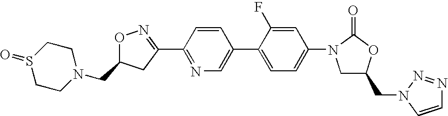

4-{[(5S)-3-(5-Bromopyridin-2-yl)-4,5-dihydroisoxazol-5-yl]methyl}thiomorpholine 1-oxide (Intermediate 18, 130 mg, 0.36 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 155 mg, 0.40 mmol), potassium carbonate (150 mg, 1.09 mmol), and tetrakis(triphenylphosphino)palladium(0) (42 mg, 0.036 mmol) were suspended in DMF (3 ml) and water (0.5 ml). The mixture was heated at 80° C. for 1 hour, allowed to cool, and filtered. The filtrate was combined with silica gel and dried in vacuo. The adsorbed material was purified by column chromatography (silica gel, 1 to 15% methanol in dichloromethane). The material thus obtained was dissolved in warm methanol (5 ml) and dichloromethane (5 ml), the solution was concentrated with heating to 5 ml, and allowed to cool, yielding a precipitate. The solids were filtered and rinsed with methanol, then diethyl ether and dried in vacuo. The title compound was thus obtained as an off-white solid (110 mg): melting point: 218-220° C.

MS (electrospray): 540 (M+1) for C₂₅H₂₆FN₇O₄S

¹H-NMR (400 MHz, DMSO-d₆) δ: 2.64-2.78 (m, 6H); 2.87 (bm, 2H); 3.01 (bq, 2H); 3.26 (dd, 1H); 3.53 (dd, 1H); 3.96 (dd, 1H); 4.29 (t, 1H); 4.86 (d, 2H); 4.94 (m, 1H); 5.19 (m, 1H); 7.42 (dd, 1H); 7.59 (dd, 1H); 7.68 (t, 1H); 7.76 (s, 1H); 7.99 (d, 1H); 8.05 (d, 1H); 8.18 (s, 1H); 8.82 (s, 1H).

### Intermediate 18: 4-{[(5S)-3-(5-Bromopyridin-2-yl)-4,5-dihydroisoxazol-5-yl]methyl}thiomorpholine 1-oxide

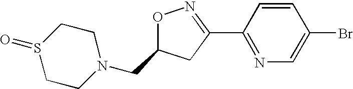

4-{[(5S)-3-(5-Bromopyridin-2-yl)-4,5-dihydroisoxazol-5-yl]methyl}thiomorpholine (Intermediate 17, 150 mg, 0.44 mmol) was dissolved in acetonitrile (4 ml) and water (1 ml) and cooled to 0° C. Solid Oxone® monopersulfate compound [2 KHSO₅.KHSO₄.K₂SO₄ (160 mg, 0.26 mmol)] was added and the cold bath was removed. The mixture was stirred for 20 minutes, suspended in acetonitrile:methanol (1:1) and filtered. The filtrate was combined with silica gel and dried in vacuo. The adsorbed material was purified by flash chromatography (silica gel, 1-50% methanol in dichloromethane) to give the title compound as an off-white solid (130 mg).

MS (electrospray): 359 (M+1) for C₁₃H₁₆N₃O₂S

¹H-NMR (400 MHz, DMSO-d₆) δ: 2.60-2.77 (m, 6H); 2.86 (bm, 2H); 3.00 (bq, 2H); 3.20 (dd, 1H); 3.48 (dd, 1H); 4.93 (m, 1H); 7.85 (d, 1H); 8.12 (d, 1H); 8.78 (s, 1H).

### EXAMPLE 9

### (5R)-3-[4-(6-{(5S)-5-[(1,1-Dioxidothiomorpholin-4-yl)methyl]-4,5-dihydroisoxazol-3-yl}pyridin-3-yl)-3-fluorophenyl]-5-(1H-1,2,3-triazol-1-ylmethyl)-1,3-oxazolidin-2-one

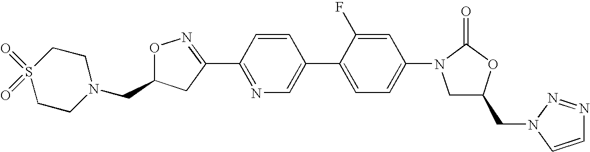

4-{[(5S)-3-(5-Bromopyridin-2-yl)-4,5-dihydroisoxazol-5-yl]methyl}thiomorpholine 1,1-dioxide (Intermediate 19, 185 mg, 0.49 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 205 mg, 0.53 mmol), potassium carbonate (200 mg, 1.45 mmol), and tetrakis(triphenylphosphino)palladium(0) (60 mg, 0.051 mmol) were suspended in DMF (3 ml) and water (0.5 ml). The mixture was heated at 80° C. for 1 hour, allowed to cool, and filtered. The filtrate was combined with silica gel and dried in vacuo. The adsorbed material was purified by column chromatography (silica gel, 1 to 10% methanol in dichloromethane). The material thus obtained was dissolved in warm ethanol (5 ml) and dichloromethane (5 ml), the solution was concentrated with heating to 5 ml, and allowed to cool, yielding a precipitate. The solids were filtered and rinsed with methanol, water, again with methanol, then diethyl ether and dried in vacuo. The title compound was thus obtained as an off-white solid (125 mg): melting point: 200-203° C.

MS (electrospray): 556 (M+1) for C₂₅H₂₆FN₇O₅S

¹H-NMR (400 MHz, DMSO-d₆) δ: 2.79 (m, 2H); 3.06 (m, 8H); 3.26 (dd, 1H); 3.54 (dd, 1H); 3.96 (dd, 1H); 4.29 (t, 1H); 4.86 (d, 2H); 4.94 (m, 1H); 5.19 (m, 1H); 7.42 (dd, 1H); 7.59 (dd, 1H); 7.69 (t, 1H); 7.76 (s, 1H); 7.99 (d, 1H); 8.06 (d, 1H); 8.18 (s, 1H); 8.82 (s, 1H).

### Intermediate 19: 4-{[(5S)-3-(5-Bromopyridin-2-yl)-4,5-dihydroisoxazol-5-yl]methyl}thiomorpholine 1,1-dioxide

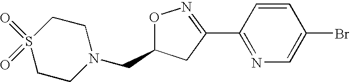

4-{[(5S)-3-(5-Bromopyridin-2-yl)-4,5-dihydroisoxazol-5-yl]methyl}thiomorpholine (Intermediate 17, 200 mg, 0.56 mmol) was dissolved in acetonitrile (5 ml) and water (2 ml) and cooled to 0° C. Solid Oxone® monopersulfate compound [2 KHSO₅.KHSO₄.K₂SO₄ (620 mg, 1.01 mmol)] was added and the cold bath was removed. The mixture was stirred for 16 hours, suspended in acetonitrile:methanol (1:1) and filtered. The filtrate was combined with silica gel and dried in vacuo. The adsorbed material was purified by flash chromatography (silica gel, 4-20% methanol in dichloromethane) to give an off-white solid (215 mg). This material was identified as the N-oxide of the title compound:

MS (electrospray): 391 (M+1) for C₁₃H₁₆N₃O₄S

Conversion to the title compound was accomplished as follows: The N-oxide prepared above (205 mg, 0.53 mmol) was combined with triphenylphosphine (250 mg, 0.95 mmol) in DMF (4 ml) and warmed to 80° C. for 15 minutes. The mixture was diluted with ethyl acetate, and washed with water. The aqueous layer was extracted with ethyl acetate and ethyl acetate: THF (1:1). The pooled organic layers were dried over sodium sulfate and evaporated. The residue was purified by flash chromatography (silica gel, 10 to 100% ethyl acetate in hexanes) to give 4-{[(5S)-3-(5-bromopyridin-2-yl)-4,5-dihydroisoxazol-5-yl]methyl}thiomorpholine 1,1-dioxide as an off-white solid (190 mg).

MS (electrospray): 375 (M+1) for C₁₃H₁₆N₃O₃S

¹H-NMR (400 MHz, DMSO-d₆) δ: 2.76 (m, 2H); 3.03 and 3.06 (2×bm, 8H); 3.20 (dd, 1H); 3.49 (dd, 1H); 4.92 (m, 1H); 7.83 (d, 1H); 8.12 (d, 1H); 8.77 (s, 1H).

### EXAMPLE 10

### (5R)-3-[3-Fluoro-4-[6-((5S)-5-{[(2-hydroxyethyl)(methyl)amino]methyl]-4,5-dihydroisoxazol-3-yl)pyridin-3-yl]phenyl}-5-(1H-1,2,3-triazol-1-ylmethyl)-1,3-oxazolidin-2-one

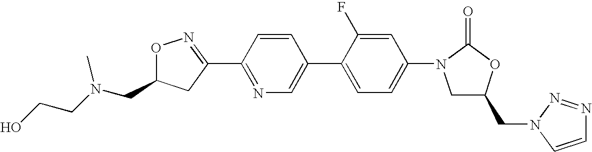

2-[{[(5S)-3-(5-Bromopyridin-2-yl)-4,5-dihydroisoxazol-5-yl]methyl}(methyl)amino]ethanol (Intermediate 20, 335 mg, 1.07 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7,420 mg, 1.08 mmol), potassium carbonate (450 mg, 3.26 mmol), and tetrakis(triphenylphosphino)palladium(0) (125 mg, 0.108 mmol) were suspended in DMF (5 ml) and water (0.5 ml). The mixture was heated at 80° C. for 40 minutes, allowed to cool, adsorbed directly onto silica gel and dried in vacuo. The adsorbed material was purified by column chromatography [silica gel, 0 to 20% methanol in (20% acetonitrile, 1% triethylamine in dichloromethane)]. The material thus obtained was crystallized from methanol, collected and rinsed with diethyl ether. The title compound was thus obtained as an off-white solid (70 mg): melting point: 153-156° C.

MS (electrospray: 496 (M+1) for C₂₄H₂₆FN₇O₄

¹H-NMR (400 MHz, DMSO-d₆) δ: 2.29 (s, 3H); 2.48 (bm, 2H); 2.63 (bm, 2H); 3.27 (dd, 1H); 3.47 (bq, 2H); 3.51 (dd, 1H); 3.96 (dd, 1H); 4.30 (t, 1H); 4.37 (bm, 1H); 4.86 (d, 2H); 4.89 (m, 1H); 5.19 (m, 1H); 7.42 (dd, 1H); 7.59 (dd, 1H); 7.69 (t, 1H); 7.76 (s, 1H); 7.99 (d, 1H); 8.05 (d, 1H); 8.18 (s, 1H); 8.81 (s, 1H).

### Intermediate 20: 2-[{[(5S)-3-(5-Bromopyridin-2-yl)-4,5-dihydroisoxazol-5-yl]methyl}(methyl)amino]ethanol

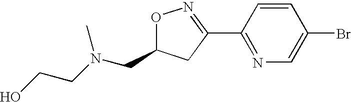

5-Bromo-2-[(5S)-5-(chloromethyl)-4,5-dihydroisoxazol-3-yl]pyridine (Intermediate 12, 0.30 g, 1.09 mmol), 2-(methylamino)ethanol (0.9 ml, 11.2 mmol), tetrabutyl ammonium iodide (2 mg, catalytic amount) and DMSO (1 ml) were combined and warmed to 110° C. for 3.5 hours. The solution was diluted with water and extracted twice with ethyl acetate. The pooled organic layers were dried over sodium sulfate and evaporated to give crude title compound as a thick yellow oil (335 mg).

MS (electrospray): 315 (M+1) for C₁₂H₁₆BrN₃O₂

### EXAMPLE 11

### (5R)-3-(3-Fluoro-4-{6-[(5R)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one

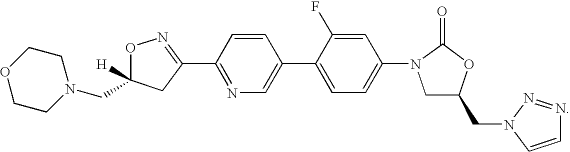

4-{[(5R)-3-(5-Bromopyridin-2-yl)-4,5-dihydroisoxazol-5-yl]methyl}morpholine (Intermediate 23, 320 mg, 0.98 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (400 mg, 1.03 mmol), potassium carbonate (Intermediate 7, 450 mg, 3.26 mmol), and tetrakis(triphenylphosphino)palladium(0) (120 mg, 0.10 mmol) were suspended in DMF (5 ml) and water (0.5 ml). The mixture was heated at 80° C. for 60 minutes, allowed to cool, and filtered. The solids were rinsed with acetonitrile and the combined filtrate was adsorbed on silica gel. The adsorbed material was purified by column chromatography (silica gel, 1 to 10% methanol in dichloromethane). The off-white solid thus obtained (430 mg) was dissolved in hot dioxane (30 ml) and treated with HCl (4M solution in dioxane, 0.25 ml, 1 mmol) to give a suspension, which was diluted with diethyl ether (50 ml) followed by filtration and rinsing with diethyl ether. The hydrochloride salt of the title compound was thus obtained as an off-white solid (400 mg): melting point: 239-245° C.

MS (electrospray): 508 (M+1) for C₂₅H₂₆FN₇O₄

¹H-NMR (400 MHz. DMSO-d₆) δ: 3.18 (bm, 2H); 3.37 (dd, 1H); 3.49 (bm, 3H); 3.77 (m, 4H); 3.96 (m, 3H); 4.30 (t, 1H); 4.86 (d, 2H); 5.19 (m, 1H); 5.32 (m, 1H); 7.42 (dd, 1H); 7.59 (dd, 1H); 7.69 (t, 1H); 7.76 (s, 1H); 8.02 (d, 1H); 8.09 (d, 1H); 8.18 (s, 1H); 8.81 (s, 1H); 10.55 (bs, 1H).

### EXAMPLE 11a

### (5R)-3-(3-Fluoro-4-{6-[(5R)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one, hydrochloride salt

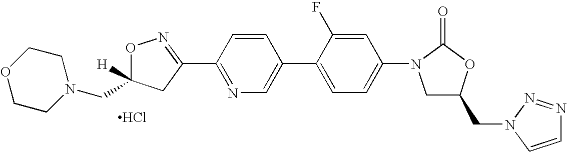

(5R)-3-(3-Fluoro-4-{6-[(5R)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one (Example 11, 430 mg, 0.85 mmol) was dissolved in hot dioxane (30 ml) and treated with HCl (4M solution in dioxane, 0.25 ml, 1 mmol) to give a suspension, which was diluted with diethyl ether (50 ml) followed by filtration and rinsing with diethyl ether. The title compound was thus obtained as an off-white solid (400 mg): melting point: 239-245° C.

MS (electrospray): 508 (M+1) for C₂₅H₂₆FN₇O₄

¹H-NMR (400 MHz, DMSO-d₆) δ: 3.18 (bm, 2H); 3.37 (dd, 1H); 3.49 (bm, 3H); 3.77 (m, 4H); 3.96 (m, 3H); 4.30 (t, 1H); 4.86 (d, 2H); 5.19 (m, 1H); 5.32 (m, 1H); 7.42 (dd, 1H); 7.59 (dd, 1H); 7.69 (t, 1H); 7.76 (s, 1H); 8.02 (d, 1H); 8.09 (d, 1H); 8.18 (s, 1H); 8.81 (s, 1H); 10.55 (bs, 1H).

### Intermediate 21: [(5R)-3-(5-bromopyridin-2-yl)-4,5-dihydroisoxazol-5-yl]methanol

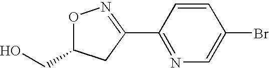

(R,S)-[3-(5-Bromopyridin-2-yl)-4,5-dihydroisoxazol-5-yl]methanol (prepared by hydrolysis of Intermediate 9, 3.1 g) was dissolved in hot methanol (25 ml), it was then separated by chiral column (Chiral Pak AS) eluting with 30% isopropanol in hexanes. The title compound [(−) isomer, 1.5 g)] which eluted first from the column was collected along with the (+) isomer (second peak, 1.18 g). Chiral HPLC analysis indicated <2% of the (+) isomer was present.

[α]_{D}=−125° (c=0.0076 g/ml in methanol).

### Alternative Preparation of Intermediate 21:

Racemic [3-(5-bromopyridin-2-yl)-4,5-dihydroisoxazol-5-yl]methyl butyrate (Intermediate 9, 140 mg, 0.43 mmol) was dissolved in acetone (10 ml), and 0.1 M potassium phosphate buffer (pH˜7) (10 ml) was added with vigorous stirring to give a clear yellow solution. PS-lipase (2 mg, Sigma cat no L-9156) was added and the mixture was gently stirred at ambient temp. for 5 hrs when HPLC analysis indicated 40% conversion. The solution was diluted with water to 40 ml and extracted with ethyl acetate (3×40 ml); the pooled organic phases were dried over sodium sulfate and evaporated. The residue was triturated with 3:1 hexane:diethyl ether (2×20 ml) to give the title compound as a white powder (35 mg). Chiral HPLC analysis indicated <0.5% of the (+) isomer was present.

[α]_{D}=−140° (c=0.01 g/ml in methanol).

### Intermediate 22: 5-Bromo-2-[(5R)-5-(chloromethyl)-4,5-dihydroisoxazol-3-yl]pyridine

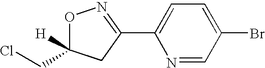

[(5R)-3-(5-Bromopyridin-2-yl)-4,5-dihydroisoxazol-5-yl]methanol (Intermediate 21, 0.274 g, 1.06 mmol) was dissolved in dichloromethane (5 ml). Triphenylphosphine (0.8 g, 3.05 mmol) and carbon tetrachloride (0.6 ml, 6.2 mmol) were added and the mixture was stirred at room temperature for 2 hours. Methanol (0.5 ml) was added, and the solution was concentrated and purified by flash chromatography (silica gel, 5 to 20% ethyl acetate in hexane) to yield the title compound as a white solid (280 mg).

¹H-NMR (300 Mt. CDCl₃) δ: 3.42-3.73 (m, 4H); 4.98-5.08 (m, 1H); 7.84 (dd, 1H); 7.90 (d, 1H); 8.65 (d, 1H).

### Intermediate 23: 4-{[(5R)-3-(5-Bromopyridin-2-yl)-4,5-dihydroisoxazol-5-yl]methyl}morpholine

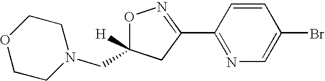

5-Bromo-2-[(5R)-5-(chloromethyl)-4,5-dihydroisoxazol-3-yl]pyridine (Intermediate 22, 0.276 g, 1.0 mmol), morpholine (0.9 ml, 10.3 mmol), tetrabutyl ammonium iodide (2 mg, catalytic amount) and DMSO (0.9 ml) were combined and warmed to 115° C. for 4 hours. The solution was diluted with water and extracted twice with ethyl acetate. The pooled organic layers were dried over sodium sulfate and evaporated to give crude title compound as a waxy yellow solid (320 mg).

MS (electrospray): 327 (M+1) for C₁₃H₁₆BrN₃O₂

### EXAMPLE 12

### (5R)-3-(3-Fluoro-4-{6-[(5S)-5-(pyrrolidin-1-ylmethyl)-4,5-dihydroisoxazol-3-yl]pyridin-3-yl}phenyl)-5-(1H-1,2,3-triazol-1-ylmethyl-1,3-oxazolidin-2-one

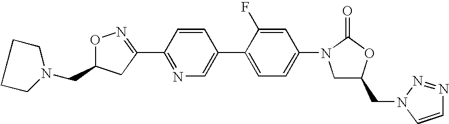

5-Bromo-2-[(5S)-5-(pyrrolidin-1-ylmethyl)-4,5-dihydroisoxazol-3-yl]pyridine (Intermediate 24, 305 mg, 0.98 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 380 mg, 0.98 mmol), potassium carbonate (400 mg, 2.9 mmol), and tetrakis(triphenylphosphino)palladium(0) (114 mg, 0.099 mmol) were suspended in DMF (5 ml) and water (0.5 ml). The mixture was heated at 80° C. for 40 minutes, allowed to cool, filtered and adsorbed on silica gel. The adsorbed material was purified by column chromatography [silica gel, (1 to 10% methanol, 0.025 to 0.5% triethylamine) in dichloromethane]. The material thus obtained was dissolved in dichloromethane (10 ml) and diluted with diethyl ether (20 ml) followed by filtration and rinsing with diethyl ether to give the free base of the title compound (200 mg). This material was dissolved in methanol: dichloromethane (1:5), HCl (4M solution in dioxane, 0.1 ml) was added, the resulting suspension was diluted with diethyl ether to give a precipitate. The solids were collected, rinsed with diethyl ether, then ethyl acetate and dried in vacuo to give the hydrochloride salt of the title compound as an off-white solid (170 mg): melting point: 260-263° C.

MS (electrospray): 492 (M+1) for C₂₅H₂₆FN₇O₃

¹H-NMR (400 MHz, DMSO-d₆) δ: 1.90 (bm, 2H); 2.02 (bm, 2H); 3.10 (bm, 2H); 3.36 (dd, 1H); 3.49 (bm, 2H); 3.61 (bm, 2H); 3.74 (dd, 1H); 3.96 (dd, 1H); 4.30 (t, 1H); 4.86 (d, 2H); 5.19 (m, 2H); 7.42 (dd, 1H); 7.59 (dd, 1H); 7.69 (t, 1H); 7.76 (s, 1H); 8.02 (d, 1H); 8.09 (d, 1H); 8.18 (s, 1H); 8.85 (s, 1H); 9.93 (bs, 1H).

### Intermediate 24: 5-Bromo-2-[(5S)-5-(pyrrolidin-1-ylmethyl)-4,5-dihydroisoxazol-3-yl]pyridine

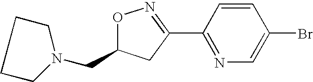

5-Bromo-2-[(5S)-5-(chloromethyl)-4,5-dihydroisoxazol-3-yl]pyridine (Intermediate 12, 0.30 g, 1.09 mmol), pyrrolidine (1 ml, 12 mmol), tetrabutyl ammonium iodide (2 mg, catalytic amount) and DMSO (1 ml) were combined and warmed to 85° C. for 16 hours. The solution was diluted with water and extracted twice with ethyl acetate. The pooled organic layers were dried over sodium sulfate and evaporated to give crude title compound as a waxy yellow solid (305 mg).

MS (electrospray): 311 (M+1) for C₁₃H₁₆BrN₃O

### EXAMPLE 13

### (5R)-3-(3-Fluoro-4-{6-[(5S)-5-(piperidin-1-ylmethyl)-4,5-dihydroisoxazol-3-yl]pyridin-3-yl}phenyl)-5-(1H-1,2,3-triazol-1-ylmethyl)-1,3-oxazolidin-2-one

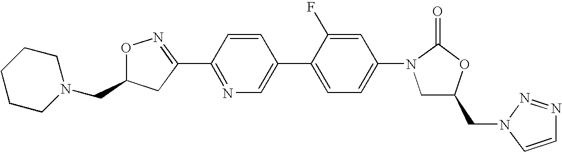

5-Bromo-2-[(5S)-5-(piperidin-1-ylmethyl)-4,5-dihydroisoxazol-3-yl]pyridine (Intermediate 25,325 mg, 1.0 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 380 mg, 0.98 mmol), potassium carbonate (400 mg, 2.9 mmol), and tetrakis(triphenylphosphino)palladium(0) (114 mg, 0.099 mmol) were suspended in DMF (5 ml) and water (0.5 ml). The mixture was heated at 80° C. for 40 minutes, allowed to cool, filtered and adsorbed on silica gel. The adsorbed material was purified by column chromatography [silica gel, (1 to 10% methanol, 0.025 to 0.5% triethylamine) in dichloromethane]. The material thus obtained was dissolved in dichloromethane (10 ml) and diluted with diethyl ether (20 ml) followed by filtration and rinsing with diethyl ether to give the free base of the title compound (200 mg). This material was dissolved in methanol:dichloromethane (1:5), HCl (4M solution in dioxane, 0.16 ml) was added, then diluted with diethyl ether to give a precipitate. The solids were collected, rinsed with diethyl ether, then ethyl acetate and dried in vacuo to give the hydrochloride salt of the title compound as an off-white solid (260 mg): melting point: 211-215° C.

MS (electrospray): 506 (M+1) for C₂₆H₂₈FN₇O₃

¹H-NMR (400 MHz, DMSO-d₆) δ: 1.17 (t, 2H); 1.65-1.87 (bm, 4H); 2.99 (bm, 1H); 3.08 (m, 1H); 3.36 (dd, 1H); 3.41 (bt, 1H); 3.51 (bd, 1H); 3.75 (dd, 1H); 3.96 (dd, 1H); 4.30 (t, 1H); 4.86 (d, 2H); 5.19 (m, 1H); 5.30 (m, 1H); 7.42 (dd, 1H); 7.59 (dd, 1H); 7.69 (t, 1H); 7.76 (s, 1H); 8.02 (d, 1H); 8.09 (d, 1H); 8.18 (s, 1H); 8.85 (s, 1H); 9.83 (bs, 1H).

### Intermediate 25: 5-Bromo-2-[(5S)-5-(piperidin-1-ylmethyl)-4,5-dihydroisoxazol-3-yl]pyridine

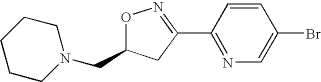

5-Bromo-2-[(5S)-5-(chloromethyl)-4,5-dihydroisoxazol-3-yl]pyridine (Intermediate 12, 0.30 g, 1.09 mmol), piperidine (1 ml, 10.1 mmol), tetrabutyl ammonium iodide (2 mg, catalytic amount) and DMSO (1 ml) were combined and warmed to 85° C. for 16 hours. The solution was diluted with water and extracted twice with ethyl acetate. The pooled organic layers were dried over sodium sulfate and evaporated to give crude title compound as a waxy yellow solid (325 mg).

MS (electrospray): 325 (M+1) for C₁₄H₁₈BrN₃O

### EXAMPLE 14

### (5R)-3-(4-{6-[(5S)-5-(3,6-Dihydropyridin-1(2H)-ylmethyl)-4,5-dihydroisoxazol-3-yl]pyridin-3-yl}-3-fluorophenyl)-5-(1H-1,2,3-triazol-1-ylmethyl)-1,3-oxazolidin-2-one

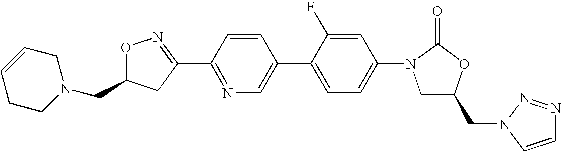

5-Bromo-2-[(5S)-5-(3,6-dihydropyridin-1(2H)-ylmethyl)-4,5-dihydroisoxazol-3-yl]pyridine (Intermediate 26, 330 mg, 1.02 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7,380 mg, 0.98 mmol), potassium carbonate (400 mg, 2.9 mmol), and tetrakis(triphenylphosphino)palladium(0) (114 mg, 0.099 mmol) were suspended in DMF (5 ml) and water (0.5 ml). The mixture was heated at 80° C. for 40 minutes, allowed to cool, filtered and adsorbed on silica gel. The adsorbed material was purified by column chromatography [silica gel, (1 to 10% methanol, 0.025 to 0.5% triethylamine) in dichloromethane]. The material thus obtained was dissolved in dichloromethane (10 ml) and diluted with diethyl ether (20 ml) followed by filtration and rinsing with diethyl ether to give the free base of the title compound (200 mg). This material was dissolved in methanol:dichloromethane (1:5), HCl (4M solution in dioxane, 0.13 ml) was added, then diluted with diethyl ether to give a precipitate. The solids were collected, rinsed with diethyl ether, then ethyl acetate and dried in vacuo to give the hydrochloride salt of the title compound as an off-white solid (230 mg): melting point: 250-255° C.

MS (electrospray): 504 (M+1) for C₂₆H₂₆FN₇O₃

¹H-NMR (400 MHz, DMSO-d₆) δ: 3.22 (bm, 1H); 3.34-3.62 (m, 6H); 3.74 (bm, 1H); 3.76 (dd, 1H); 3.88 (bm, 1H); 3.96 (dd, 1H); 4.30 (t, 1H); 4.86 (d, 2H); 5.19 (m, 1H); 5.31 (m, 1H); 5.74 (m, 1H); 5.93 (m, 1H); 7.42 (dd, 1H); 7.59 (dd, 1H); 7.69 (t, 1H); 7.76 (s, 1H); 8.02 (d, 1H); 8.10 (d, 1H); 8.18 (s, 1H); 8.85 (s, 1H); 10.11 (bs, 1H).

### Intermediate 26: 5-Bromo-2-[(5S)-5-(3,6-dihydropyridin-1(2H)-ylmethyl)-4,5-dihydroisoxazol-3-yl]pyridine

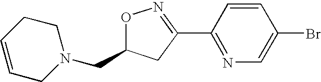

5-Bromo-2-[(5S)-5-(chloromethyl)-4,5-dihydroisoxazol-3-yl]pyridine (Intermediate 12, 0.30 g, 1.09 mmol), 1,2,3,6-tetrahydropyridine (1 ml, 10.9 mmol), tetrabutyl ammonium iodide (2 mg, catalytic amount) and DMSO (1 ml) were combined and warmed to 90° C. for 16 hours. The solution was diluted with water and extracted twice with ethyl acetate. The pooled organic layers were dried over sodium sulfate and evaporated to give crude title compound as a waxy yellow solid (330 mg).

MS (electrospray): 323 (M+1) for C₁₄H₁₆BrN₃O

### EXAMPLE 15

### tert-Butyl {[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}carbamate

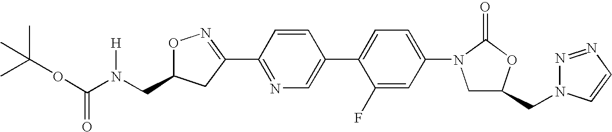

tert-Butyl {[3-(5-bromopyridin-2-yl)-4,5-dihydroisoxazol-5-yl]methyl}carbamate (Intermediate 30, 408 mg, 1.1 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 427 mg, 1.1 mmol), potassium carbonate (456 mg, 3.3 mmol), and tetrakis(triphenylphosphino)palladium(0) (64 mg, 0.06 mmol) were suspended in DMF (7.2 ml) and water (0.72 ml). The mixture was heated at 85° C. for 1 hour under nitrogen. The reaction mixture was filtered then purified by column chromatography (silica gel, 100% ethyl acetate to 50% acetonitrile in ethyl acetate). The title compound was thus obtained as yellow crystalline solid (227 mg): melting point: 228-230° C.

MS (electrospray): 536 (M+1) for C₂₆H₂₈FN₇O₅

¹H-NMR (300 MHz, DMSO-d6) δ: 1.37 (s, 9H); 3.09-3.33 (m, 2H); 3.33 (m, 2H) 3.45-3.55 (m, 1H); 3.91-3.96 (m, 1H); 4.264-4.32 (m, 1H); 4.71-4.83 (m, 1H) 4.84-4.87 (m, 2H); 5.14-5.22 (m, 1H); 7.40-7.42 (dd, 1H); 7.56-7.60 (dd, 1H); 7.66-7.72 (m, 1H); 7.77 (s, 1H); 7.98-8.00 (d, 1H); 8.04-8.07 (d, 1H); 8.18 (s, 1H); 8.81 (s, 1H).

### Intermediate 27: [(5S)-3-(5-Bromopyridin-2-yl)-4,5-dihydroisoxazol-5-yl]methyl methanesulfonate

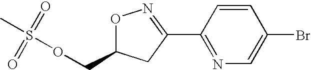

[(5S)-3-(5-Bromopyridin-2-yl)-4,5-dihydroisoxazol-5-yl]methanol (Intermediate 11, 3 g, 11.7 mmol) was added to anhydrous dichloromethane (15 ml) followed by addition of triethylamine (2.27 ml, 16.3 mmol). The solution was allowed to cool to 0° C. followed by drop wise addition of methane sulfonyl chloride (1.08 ml, 1.4 mmol). The reaction was allowed to stir for 2 hours at 0° C. and then aqueous sodium bicarbonate (20 ml) was added. After further extraction with dichloromethane (2×20 ml), the organic layers were combined, dried over sodium sulfate, and concentrated in vacuo to give the desired product (4.8 g).

¹H-NMR (DMSO-d₆) δ: 3.08 (s, 3H); 3.27 (dd, 1H); 3.47 (dd, 1H); 4.37 (m, 2H); 5.02 (m, 1H); 7.53 (m, 4H).

### Intermediate 28: 2-[(5S)-5-(Azidomethyl)-4,5-dihydroisoxazol-3-yl]-5-bromopyridine

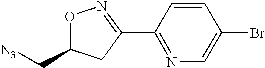

[(5S)-3-(5-Bromopyridin-2-yl)-4,5-dihydroisoxazol-5-yl]methyl methanesulfonate (Intermediate 27, 1.5 g, 4.6 mmol) was added to dimethyl formamide (8 ml) followed by addition of sodium azide (0.6 g, 9.0 mmol). The mixture was heated to 75° C. for six hours and then added to aqueous sodium chloride (10 ml) followed by extraction with ethyl acetate. (3×20 ml). The organic layers were combined, dried over sodium sulfate, and concentrated in vacuo to yield the desired product (1.1 g).

¹H-NMR (DMSO-d₆) δ: 3.25 (dd, 1H); 3.53 (dd, 1H); 3.61 (m, 2H); 4.96 (m, 1H); 7.65 (d, 2H); 7.71 (d, 2H).

### Intermediate 29: {[(5S)-3-(5-Bromopyridin-2-yl)-4,5-dihydroisoxazol-5-yl]methyl}amine

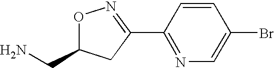

2-[(5S)-5-(Azidomethyl)-4,5-dihydroisoxazol-3-yl]-5-bromopyridine (Intermediate 28, 2 g, 7.1 mmol) was dissolved in dichloromethane:methanol:water 3.5:2:1 (13 ml) followed by addition of 8 grams of triphenylphosphine bound polystyrene resin (Argonaut Technologies, Inc. Foster City, Calif. USA) (1.6 mmol per gram). The mixture was stirred at room temperature for 16 hours and filtered. The resin was washed with dichloromethane (20 ml) and methanol (10 ml) and then the solvents were concentrated in vacuo to give the desired product (1.45 g).

¹H-NMR (DMSO-d₆) δ: 3.75 (m, 2H); 3.25 (dd, 1H); 3.44 (dd, 1H); 4.69 (m, 1H); 7.62 (d, 2H); 7.68 (d, 2H).

### Intermediate 30: tert-Butyl {[3-(5-bromopyridin-2-yl)-4,5-dihydroisoxazol-5-yl]methyl}carbamate

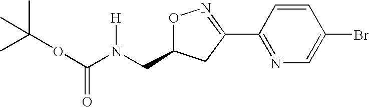

{[(5S)-3-(5-Bromopyridin-2-yl)-4,5-dihydroisoxazol-5-yl]methyl}amine (400 mg, 1.56 mmol) was dissolved in dichloromethane (4 ml). Saturated aqueous sodium bicarbonate (4 ml) was added to the stirring reaction mixture followed by di-tert-butyl dicarbonate (1 g, 4.59 mmol). The reaction mixture was stirred at room temperature for 16 hours. Dichloromethane and water were added and the layers were separated. The dichloromethane layer was dried over sodium sulfate, evaporated and purified via chromatography (silica gel, 10 to 50% ethyl acetate in hexanes). Evaporation of the product containing fractions and drying in vacuo yielded the title compound (408 mg).

MS (electrospray): 301 (M+1) C₁₄H₁₈BrN₃O₃

### REFERENCE EXAMPLE 16

### (5R)-3-(4-{6-[(5S)-5-(Aminomethyl)-4,5-dihydroisoxazol-3-yl]pyridin-3-yl}-3-fluorophenyl)-5-(1H-1,2,3-triazol-1-ylmethyl)-1,3-oxazolidin-2-one

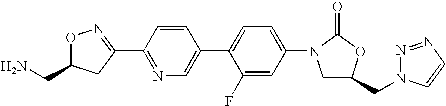

tert-Butyl {[(5S)-3-(5-{2-fluoro-4-[(SR)-2-oxo-5-(1H-1,2,3-triazol-1-ylmethyl)-1,3-oxazolidin-3-yl]phenyl}pyridin-2-yl)-4,5-dihydroisoxazol-5-yl]methyl}carbamate (Intermediate 15, 150 mg, 0.29 mmol), was dissolved in DMF/Dioxane (5 ml) followed by slow addition of 4M HCl in dioxane (2.4 ml) and allowed to stir for 18 hours. The product was precipitated with acetonitrile/ether and filtered under nitrogen. The yellow solid was triturated using ether. The title compound was thus obtained as yellow crystalline solid (120 mg): melting point: 119-122° C.

MS (electrospray): 438.2 (M+1) C₂₁H₂₀FN₇O₃

¹H-NMR (300 MHz. DMSO-d₆) δ: 2.91-3.32 (m, 2H); 3.33-3.45 (m, 1H); 3.56-3.71 (m, 2H); 4.26-4.32 (m, 1H); 4.84-4.87 (m, 2H); 5.06 (m, 1H); 5.14-5.22 (m, 1H); 7.40-7.42 (dd, 1H); 7.56-7.60 (dd, 1H); 7.66-7.72 (m, 1H); 7.77 (s, 1H); 7.95-8.01 (d, 1H); 8.04-8.07 (d, 1H); 8.36 (s, 1H); 8.85 (s, 1H).

### EXAMPLE 17

### N¹-{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-N²,N²-dimethylglycinamide hydrochloride

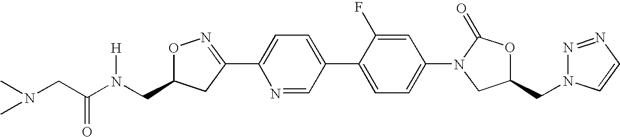

N¹-{[(5S)-3-(5-Bromopyridin-2-yl)-4,5-dihydroisoxazol-5-yl]methyl}-N²,N²-dimethylglycinamide (Intermediate 31,450 mg, 1.3 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7, 512 mg, 1.3 mmol), potassium carbonate (539 mg, 3.9 mmol), and tetrakis(triphenylphosphino)palladium(0) (65 mg, 0.05 mmol) were suspended in DMP (8.1 ml) and water (0.81 ml). The mixture was heated at 85° C. for 1 hour under nitrogen. After reaction completion, the reaction mixture was allowed to cool to room temperature and filtered. The filter cake was washed with methanol. The filtrate was concentrated down and purified by column chromatography (silica gel, 100% ethyl acetate to 30% methanol in ethyl acetate) which yielded N¹-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-N²,N²-dimethylglycinamide (0.435 g).

This material (0.250 g, 0.5 mmol) was dissolved in DMF (3 ml), methanol (10 ml) dichloromethane (5 ml) and acetonitrile (10 ml) followed by addition of 4M HCl in dioxane (0.15 ml, 0.6 mmol) and allowed to stir at room temperature for 25 minutes. Ether (30 ml) was added to the reaction mixture. The solid formed from the ether precipitation was filtered to give the title compound as the hydrochloride salt. Melting point: 202-204° C.

MS (electrospray): 523 (M+1) for C₂₅H₂₇FN₈O₄

¹H-NMR (300 MHz, DMSO-d6) 2.43-2.55 (m, 6H); 3.21-3.35 (m, 1H); 3.36-3.48 (m, 2H); 3.51-3.68 (m, 1H); 4.22 (m, 2H), 4.30-4.33 (m. 2H), 4.86 (s, 2H); 5.14-5.24 (m, 1H); 7.42-7.44 (dd, 1H); 7.57-7.62 (dd, 1H); 7.67-7.72 (m, 1H); 7.78 (s, 1H); 7.98-8.01 (d, 1H); 8.05-8.08 (d, 1H); 8.19 (s, 1H); 8.82 (s, 1H); 9.78-9.89 (s, 1H).

### Intermediate 31: N¹-{[(5S)-3-(5-Bromopyridin-2-yl)-4,5-dihydroisoxazol-5-yl]methyl}-N²,N²-dimethylglycinamide

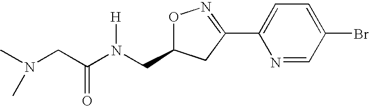

{[(5S)-3-(5-Bromopyridin-2-yl)-4,5-dihydroisoxazol-5-yl]methyl}amine (Intermediate 29, 410 mg, 1.6 mmol) was dissolved in anhydrous DMF (5 ml) under nitrogen. Diisopropylethyl amine (0.836 ml, 4.8 mmol) was added to the reaction mixture and stirred at room temperature. In a separate flask, HATU, (670 mg, 1.8 mmol) was dissolved in anhydrous DMF (2 ml). Dimethyl glycine (165 mg, 1.6 mmol) was added to the reaction mixture containing HATU and allowed to stir for 20 minutes. The amine and diisopropylethyl amine mixture was slowly added to the HATU and dimethyl glycine mixture. The reaction mixture was allowed to stir for 18 hours at room temperature under nitrogen. The reaction was worked up using dichloromethane and water. The organic layer was washed with saturated sodium chloride, dried over sodium sulfate, evaporated and purified via chromatography (silica gel, 1 to 20% methanol in ethyl acetate). Evaporation of the product containing fractions and drying in vacuo yielded the title compound as a solid (450 mg).

MS (electrospray): 341.0 (M+1) for C₉H₁₀BrN₃O

### EXAMPLE 18

### tert-Butyl N-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-N-methylglycinate

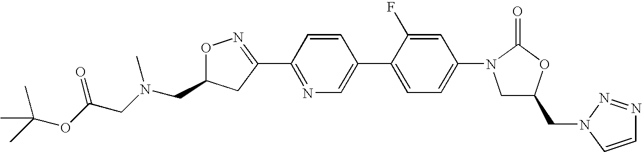

tert-Butyl N-{[(5S)-3-(5-bromopyridin-2-yl)-4,5-dihydroisoxazol-5-yl]methyl}-N-methylglycinate (Intermediate 32, 275 mg, 0.72 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 7,340 mg, 0.88 mmol), potassium carbonate (300 mg, 2.2 mmol), and tetrakis(triphenylphosphino)palladium(0) (88 mg, 0.076 mmol) were suspended in DMF (5 ml) and water (0.5 ml). The mixture was heated at 80° C. for 1 hour, allowed to cool, filtered and adsorbed on silica gel. The adsorbed material was purified by column chromatography (silica gel, 0.5 to 5% methanol in dichloromethane). The material thus obtained was dissolved in hot methanol (3 ml) and diluted with diethyl ether (10 ml) followed by filtration and rinsing with diethyl ether to give the title compound as an off-white solid (254 mg): melting point: 180° C.

MS (electrospray): 566 (M+1) for C₂₈H₃₂FN₇O₅

¹H-NMR (400 MHz, DMSO-d₆) δ: 1.43 (s, 9H); 3.34 (bm, 6M); 3.59 (bs, 1H); 3.96 (dd, 1H); 4.29 (t, 1H); 4.86 (d, 2H); 5.19 (m, 2H); 7.42 (dd, 1H); 7.59 (dd, 1H); 7.69 (t, 1H); 7.77 (s, 1H); 8.00 (d, 1H); 8.07 (d, 1H); 8.18 (s, 1H); 8.83 (s, 1H).

### Intermediate 32: tert-Butyl N-{[(5S)-3-(5-bromopyridin-2-yl)-4,5-dihydroisoxazol-5-yl]methyl}-N-methylglycinate

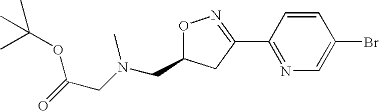

5-Bromo-2-[(5S)-5-(chloromethyl)-4,5-dihydroisoxazol-3-yl]pyridine (Intermediate 12, 0.40 g, 1.45 mmol), sarcosine tert-butyl ester hydrochloride salt (1.3 g, 7.14 mmol), diisopropylethyl amine (2.6 ml, 14.9 mol), tetrabutyl ammonium iodide (2 mg, catalytic amount) and DMSO (3 ml) were combined and warmed to 105° C. for 2 days. The solution was diluted with water and extracted twice with ethyl acetate. The pooled organic layers were dried over sodium sulfate, evaporated and purified by chromatography (silica gel, 10 to 50% ethyl acetate in hexane) to obtain the title compound as a thick oil (275 mg).

MS (electrospray): 385 (M+1) for C₁₆H₂₂BrN₃O₃

### EXAMPLE 19

### N-{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-N-methylglycine

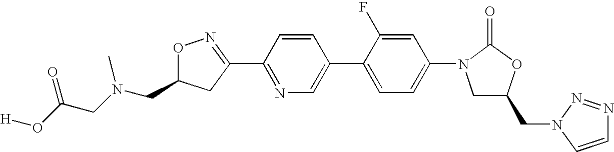

tert-Butyl N-{[(5S)-3-(5-{2-fluoro-4-[(SR)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-N-methylglycinate (Example 18, 210 mg, 0.37 mmol) was dissolved in acetic acid (6 ml), isopropanol (2 ml) and water (0.5 ml). To the solution was added HCl (4M solution in dioxane, 1 ml, 4 mmol). The solution was stirred at room temperature for 2 hours giving an oily precipitate. Concentrated HCl (0.5 ml, 6 mmol) was added to give a clear solution, which was stirred at room temperature overnight again giving an oily precipitate. The mixture was warmed at 80° C. (giving a clear solution) for 3 hours then concentrated to give a thick yellow oil. The oil was dissolved in water (5 ml) and passed through a disposable extraction column of C18 silica gel (2 g), eluting further with water, then acetonitrile/water. The combined eluent was concentrated to give a yellow residue, which was crystallized from ethanol. The resulting solid was collected, rinsed with cold ethanol and ether, then dried under vacuum at 50° C. to give the title compound as an off-white solid (155 mg): melting point: 212-218° C.

MS (electrospray): 510 (M+1) for C₂₄H₂₄FN₇O₅

¹H-NMR (400 MHz, DMSO-d₆) δ: 2.92 (s, 3H); 3.37 (dd, 1H); 3.47 (bm, 2H); 3.73 (dd, 1H); 3.96 (dd, 1H); 4.12 (bm, 2H); 4.30 (t, 1H); 4.86 (d, 2H); 5.19 (m, 1H); 5.26 (m, 1H); 7.42 (dd, 1H); 7.59 (dd, 1H); 7.69 (t, 1H); 7.76 (s, 1H); 8.01 (d, 1H); 8.09 (d, 1H); 8.18 (s, 1H); 8.84 (s, 1H).

### EXAMPLE 20

### N-{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-prolinamide hydrochloride

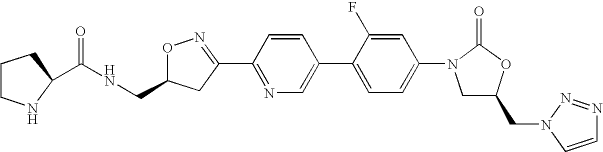

(5R)-3-(4-{6-[(5S)-5-(Aminomethyl)-4,5-dihydroisoxazol-3-yl]pyridin-3-yl}-3-fluorophenyl)-5-(1H-1,2,3-triazol-1-ylmethyl)-1,3-oxazolidin-2-one (Reference Example 16, 300 mg, 0.69 mmol), was dissolved in anhydrous DMF (5 ml) while stirring under nitrogen. HATU (286 mg, 0.75 mmol) was added to the reaction mixture followed by the addition of BOC-L-proline (147 mg, 0.69 mmol). Diisopropylethylamine was added (358 ml, 2.03 mmol) and the reaction was allowed to stir for 2 hours at room temperature. After reaction completion, the white precipitate formed during the reaction was filtered to give tert-butyl (2S)-2-[({[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}amino)carbonyl]pyrrolidine-1-carboxylate (153 mg, >90% purity by LCMS). The filtrate was purified by chromatography (silica gel; elution with 10% methanol in ethyl acetate to 45% methanol in ethyl acetate) to give more tert-butyl (2S)-2-[({[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}amino)carbonyl]pyrrolidine-1-carboxylate as a white solid (106 mg) and added to the precipitate solid to yield 259 mg, 0.41 mmol. The solid was dissolved in DMF (1 ml) to which was added 4M HCl in dioxane (4 ml). The reaction mixture was stirred at room temperature for 18 hours then heated to 60° C. for 1 hour while monitoring by HPLC. The reaction mixture was cooled down to room temperature and the product was precipitated out with acetonitrile. The solid was filtered and washed with ether to give the title product as a yellow solid (100 mg) after drying at 40° C. under vacuum for 32 hours.

Melting point: 246-249° C.

MS (electrospray): 535.5 (M+1) for C₂₆H₂₇FN₈O₄

¹H-NMR (300 MHz. DMSO-d₆) δ: (1.58-1.80 m, 1H); 2.25 (m, 1H); 3.18-3.29 (m, 1H); 3.14-3.57 (m, 6H); 3.98 (m, 1H), 4.14 (m, 1H), 4.22 (m, 2H), 4.26-4.33 (m. 2H), 4.86 (s, 2H); 5.17-5.20 (m, 1H); 7.42-7.44 (dd, 1H); 7.57-7.72 (m, 1H); 7.76 (s, 1H); 7.98-8.08 (m, 1H); 8.19 (s, 1H); 8.43-8.59 (m, 1H); 8.82 (m, 2H); 9.77 (s, 1H).

### EXAMPLE 21

### N¹-{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-D-valinamide hydrochloride

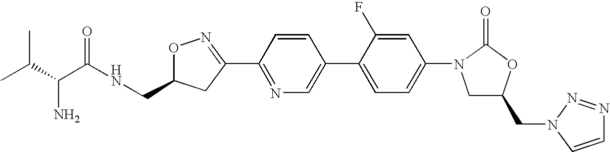

(5R)-3-(4-{6-[(5S)-5-(Aminomethyl)-4,5-dihydroisoxazol-3-yl]pyridin-3-yl}-3-fluorophenyl)-5-(1H-1,2,3-triazol-1-ylmethyl)-1,3-oxazolidin-2-one (Reference Example 16, 300 mg, 0.69 mmol), was dissolved in anhydrous DMF (5 ml) while stirring under nitrogen. HATU (286 mg, 0.75 mmol) was added to the reaction mixture followed by the addition of BOC-D-valine (150 mg, 0.69 mmol). Diisopropylethylamine was added (358 ml, 2.03 mmol) and the reaction was allowed to stir for 2 hours at room temperature. After reaction completion, the reaction mixture was purified by chromatography (silica gel; elution with 10% methanol in ethyl acetate to 45% methanol in ethyl acetate) to give tert-butyl {(1R)-1-[({[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}amino)carbonyl]-2-methylpropyl}carbamate as a white solid (221 mg, 0.35 mmol). The solid was dissolved in DMF (1 ml) to which was added 4M HCl in dioxane (4 ml). The reaction mixture was stirred at room temperature for 18 hours then heated to 60° C. for 1 hour while monitoring by HPLC. The reaction mixture was cooled to room temperature and the product was precipitated out with acetonitrile. The solid was filtered and washed with ether to give the title product as a yellow solid (132 mg) after drying at 40° C. under vacuum for 32 hours.

Melting point: 130-134° C.

MS (electrospray): 537.6 (M+1) for C₂₆H₂₉FN₈O₄

¹H-NMR (300 MHz, DMSO-d₆) δ: 0.93 (m, 6H); 2.07 (m, 1H); 2.25 (m, 1H), 2.71 (s, 1H); 2.85 (s, 1H); 3.23-3.45 (m, 1H); 3.45-3.60 (m, 1H), 3.98-3.98 (m, 1H), 4.22 (m, 2H), 4.26-4.33 (m. 2H), 4.86 (s, 2H); 5.17-5.20 (m, 1H); 7.42-7.44 (dd, 1H); 7.57-7.72 (m, 1H); 7.76 (s, 1H); 7.98-8.08 (m, 1H); 8.19 (s, 1H); 8.43-8.59 (m, 1H); 8.82 (m, 2H)

### EXAMPLE 22

### N¹-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-alaninamide hydrochloride

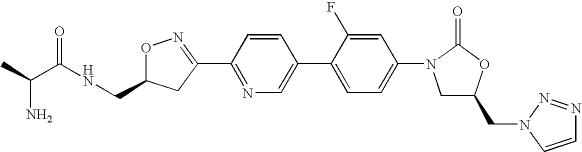

(5R)-3-(4-{6-[(5S)-5-(Aminomethyl)-4,5-dihydroisoxazol-3-yl]pyridin-3-yl}-3-fluorophenyl)-5-(1H-1,2,3-triazol-1-ylmethyl)-1,3-oxazolidin-2-one (Reference Example 16, 300 mg, 0.69 mmol), was dissolved in anhydrous DMF (5 ml) while stirring under nitrogen. HATU (286 mg, 0.75 mmol) was added to the reaction mixture followed by the addition of BOC-L-alanine (130 mg, 0.69 mmol). Diisopropylethylamine was added (358 ml, 2.03 mmol) and the reaction was allowed to stir for 2 hours at room temperature. After reaction completion, the reaction mixture was purified by chromatography (silica gel; elution with 10% methanol in ethyl acetate to 45% methanol in ethyl acetate) to give tert-butyl [(1S)-2-({[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}amino)-1-methyl-2-oxoethyl]carbamate as a white solid (351 mg, 0.58 mmol). The solid was dissolved in DMF (1 ml) to which was added 4M HCl in dioxane (4 ml). The reaction mixture was stirred at room temperature for 18 hours then heated to 60° C. for 1 hour while monitoring by HPLC. The reaction mixture was cooled to room temperature and the product was precipitated out with acetonitrile. The solid was filtered and washed with ether to give the title compound as a yellow solid (163 mg) after drying at 40° C. under vacuum for 32 hours.

Melting point: 242-244° C.

MS (electrospray: 509.5 (M+1) for C24H₂₅FN₈O₄

¹H-NMR (300 MHz. DMSO-d₆) δ: 1.21 (m. 3H); 3.23-3.45 (m, 1H); 3.45-3.60 (m, 1H), 3.98-3.98 (m, 1H), 4.22 (m, 2H), 4.26-4.33 (m. 2H), 4.86 (s, 2H); 5.17-5.20 (m, 1H); 7.42-7.44 (dd, 1H); 7.57-7.72 (m, 1H); 7.76 (s, 1H); 7.98-8.08 (m, 1H); 8.19 (s, 1H); 8.43-8.59 (m, 1H); 8.82 (m, 2H)

### EXAMPLE 23

### N¹-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-N¹,N²,N²-trimethylglycinamide

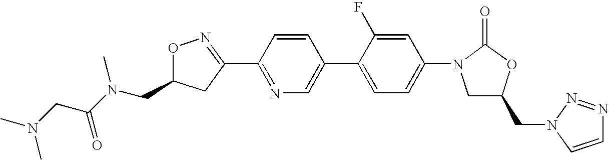

HATU, (285 mg, 0.75 mmol) was dissolved in anhydrous DMF (5 ml). Dimethyl glycine (103 mg, 1 mmol) was added and the suspension was stirred for 60 minutes to give a clear solution. (5R)-3-[3-fluoro-4-(6-{(5S)-5-[(methylamino)methyl]-4,5-dihydroisoxazol-3-yl}pyridin-3-yl)phenyl]-5-(1H-1,2,3-triazol-1-ylmethyl)-1,3-oxazolidin-2-one, (Example 2, 0.225 g, 0.50 mmol), and diisopropylethylamine (0.3 ml, 1.73 mmol) were added and the reaction mixture was allowed to stir for 3.5 hours at room temperature. The reaction was adsorbed directly onto silica gel and purified via chromatography (silica gel, 0.5% methanol/0.05% triethylamine to 10% methanol/1% triethylamine in dichloromethane). Evaporation of the product containing fractions and drying in vacuo gave a crude product residue (160 mg). A portion of this material (50 mg) was further purified by preparative reverse-phase HPLC (C18, 5-95% acetonitrile/water with 0.1% trifluoroacetic acid). The product containing fractions were evaporated, dissolved in methanol (5 ml), treated with HCl (0.2M in dioxane, 0.6 ml, 0.12 mmol) and concentrated. The sample was dissolved in methylene chloride/methanol, then diluted with ethyl acetate/ether to give a precipitate. The precipitate was collected, rinsed with ether and dried in vacuo to give the hydrochloride salt of the title compound as an off-white solid (27 mg), melting point: 122-132° C.

MS (electrospray): 537 (M+1) for C₂₆H₂₉FN₈O₄

¹H-NMR (400 MHz, DMSO-d₆) δ: 2.80 (m, 6H); 3.02 (s, 3H); 3.27 (dd, 1H); 3.52-3.68 (m, 4H); 3.96 (dd, 1H); 4.30 (m, 2H); 4.86 (d, 2H); 4.99 (m, 1H); 5.19 (m, 1H); 7.42 (dd, 1H); 7.59 (dd, 1H); 7.68 (t, 1H); 7.76 (s, 1H); 8.01 (d, 1H); 8.07 (d, 1H); 8.18 (s, 1H); 8.82 (s, 1H); 9.57 (bs, 1H).

### EXAMPLE 24

### N-{2-[{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}(methyl)amino]-2-oxoethyl}-N-methylglycine

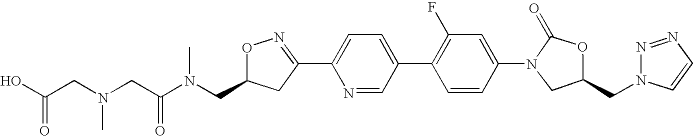

tert-Butyl N-{2-[{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}(methyl)amino]-2-oxoethyl}-N-methylglycinate (Intermediate 33, 70 mg) was dissolved in trifluoroacetic acid (5 ml), then warmed to 55° C. for 1 hour. The solution was concentrated to dryness, dissolved in methanol (1 ml), then ether (15 ml) was added and the resulting suspension was sonicated. The solid was collected, rinsed with ether and dried under vacuum to give the title compound as an off-white solid (55 mg).

Melting point 135-145° C.

MS (electrospray): 581 (MH⁺) for C₂₇H₂₉FN₈O₆

¹H-NMR (400 MHz, DMSO-d₆) δ: 2.79 (bs, 3H); 2.99 and 3.03 (2×s, 3H); 3.19-3.33 (m, 2H); 3.52-3.69 (m, 3H); 3.94 (m, 3H); 4.21 (bs, 1H); 4.29 (t, 1H); 4.86 (d, 2H); 4.95-5.08 (m, 1H); 5.19 (m, 1H); 7.42 (d, 1H); 7.58 (d, 1H); 7.68 (t, 1H); 7.76 (s, 1H); 8.02 (t, 1H); 8.07 (m, 1H); 8.18 (s, 1H); 8.82 (2×s, 1H).

### Intermediate 33: tert-Butyl N-{2-[{[(5S)-3-(5-[2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}(methyl)amino]-2-oxoethyl]-N-methylglycinate

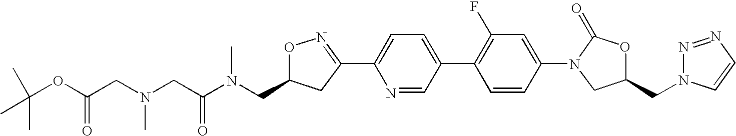

(5R)-3-[3-Fluoro-4-(6-{(5S)-5-[(methylamino)methyl]-4,5-dihydroisoxazol-3-yl}pyridin-3-yl)phenyl]-5-(1H-1,2,3-triazol-1-ylmethyl)-1,3-oxazolidin-2-one (Example 2, 0.198 g, 0.44 mmol), N-(2-tert-butoxy-2-oxoethyl)-N-methylglycine sodium salt (Intermediate 33A, 0.200 g, 0.89 mmol), N-[3-(dimethylamino)propyl]-N′-ethylcarbodiimide hydrochloride (0.200 g, 1.04 mmol), 4-dimethylaminopyridine (5 mg, catalytic amount) and DMF (5 ml) were combined to give a suspension. The mixture was stirred for 2 hours at room temperature, diluted with ethyl acetate, washed with water, then brine, dried over sodium sulfate and evaporated. The material was purified by column chromatography (silica gel; 0.5-5% MeOH in dichloromethane) to yield the title compound as a thick oil (70 mg).

MS (electrospray): 637 (MH⁺) for C₃₁H₃₇FN₈O₆

### Intermediate 33A: N-(2-tert-butoxy-2-oxoethyl)-N-methylglycine sodium salt

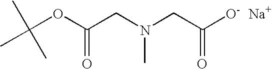

Sarcosine ethyl ester hydrochloride (3.2 g, 20.7 mmol) was combined with DMF (10 ml) and diisopropylethyl amine (3.6 ml, 20.7 mmol) and cooled to 0° C. t-Butyl bromoacetate (3 ml, 20.3 mmol) was added giving a suspension after several minutes of stirring at 0° C. After 20 minutes the cold bath was removed and the mixture was stirred at room temperature for 1 hour, then diluted with saturated sodium bicarbonate and extracted with ethyl acetate. The organic layer was dried over sodium sulfate and evaporated to yield crude N-(2-tert-butoxy-2-oxoethyl)-N-methylglycine ethyl ester as a clear light yellow liquid (2.76 g). The crude ethyl ester (1.08 g, 4.67 mmol) was dissolved in ethanol (6 ml). Aqueous sodium hydroxide (5M, 1 ml, 5 mmol) was added and the mixture was stirred at room temperature for 1 day yielding a suspension. The solid was collected and dried under vacuum to yield the title compound as a white solid (260 mg).

¹H-NMR (400 MHz, CD₃OD) δ: 1.44 (s, 9H); 2.39 (s, 3H); 3.14 (s, 2H); 3.57 (s, 2H).

### EXAMPLE 25

### N-[2-({[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}amino)-2-oxoethyl]glycine

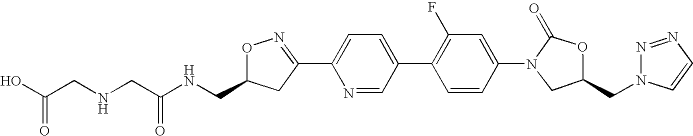

tert-Butyl N-(tert-butoxycarbonyl)-N-[2-({[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}amino)-2-oxoethyl]glycinate (Intermediate 34, 157 mg, 0.22 mmol) was dissolved in trifluoroacetic acid (8 ml), then warmed to 45° C. for 1 hour. The solution was concentrated to dryness and the residue was dissolved in water (1 ml). The product solution was filtered through a small column (2 g C18 reverse phase silica, 0 to 100% acetonitrile in water) and the eluent was evaporated. Ethyl acetate (5 ml) was added to the residue followed by sonication to yield a suspension. The solid was collected, rinsed with ether and dried under vacuum to give the title compound as an off-white solid (110 mg).

Melting point 200-210° C.

MS (electrospray): 553 (MH⁺) for C₂₅H₂₅FN₈O₆

¹H-NMR (400 MHz. DMSO-d₆) δ: 3.26 (dd, 1H); 3.43 (m, 2H); 3.55 (dd, 1H); 3.72 (s, 2H); 3.80 (s, 2H); 3.96 (dd, 1H); 4.29 (t, 1H); 4.84 (m, 2H); 4.86 (d, 2H); 5.19 (m, 1H); 7.42 (dd, 1H); 7.59 (dd, 1H); 7.68 (t, 1H); 7.76 (s, 1H); 8.00 (d, 1H); 8.07 (d, 1H); 8.18 (s, 1H); 8.70 (t, 1H); 8.82 (s, 1H).

### Intermediate 34: tert-Butyl N-(tert-butoxycarbonyl)-N-[2-({[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}amino)-2-oxoethyl]glycinate

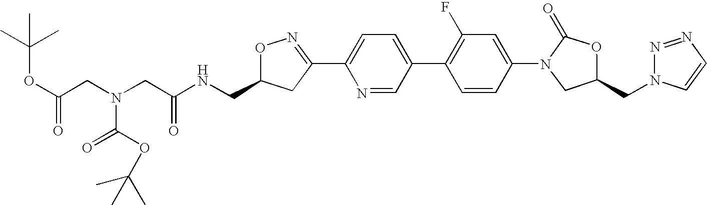

N-(tert-Butoxycarbonyl)-N-(2-tert-butoxy-2-oxoethyl)glycine [Tetrahedron Letters 1998, 39, 253] (250 mg, 0.90 mmol), (5R)-3-(4-{6-[(5S)-5-(aminomethyl)-4,5-dihydroisoxazol-3-yl]pyridin-3-yl}-3-fluorophenyl)-5-(1H-1,2,3-triazol-1-ylmethyl)-1,3-oxazolidin-2-one (Reference Example 16, 197 mg, 0.45 mmol), N-[3-(dimethylamino)propyl]-N′-ethylcarbodiimide hydrochloride (180 mg, 0.94 mmol), 4-dimethylaminopyridine (5 mg, catalytic amount) and DMF (4 ml) were combined and stirred at room temperature for 30 minutes. The mixture was diluted with ethyl acetate, washed with water, then saturated sodium chloride, dried over sodium sulphate and evaporated. The material was purified by column chromatography (silica gel; 0.5-4% MeOH in dichloromethane) to yield the crude title compound as a light yellow solid (157 mg).

MS (electrospray): 709 (MH⁺) for C₃₄H₄₁FN₈O₈

### EXAMPLE 26

### (5R)-3-(3-Fluoro-4-{6-[(5S)-5-(1H-imidazol-1-ylmethyl)-4,5-dihydroisoxazol-3-yl]pyridin-3-yl}phenyl)-5-(1H-1,2,3-triazol-1-ylmethyl)-1,3-oxazolidin-2-one

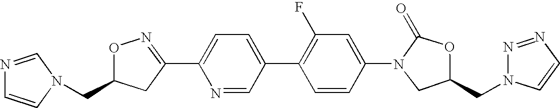

5-Bromo-2-[(5S)-5-(1H-imidazol-1-ylmethyl)-4,5-dihydroisoxazol-3-yl]pyridine (Intermediate 35, 100 mg, 0.33 mmoles), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (126 mg, 0.33 mmol), potassium carbonate (137 mg, 1 mmol) and tetrakis (triphenylphosphine) palladium (0) (19 mg, 0.017 mmol) were combined in DMF (1.8 ml) and distilled water (0.18 ml) then heated to 80° C. for 30 minutes. The reaction mixture was adsorbed directly onto silica gel then purified by column chromatography (silica gel; 0-50% MeOH in dichloromethane) to yield the title compound as an off-white solid (70 mg). Melting point: 239-242° C.

MS (electrospray): 489 (M+1) for C₂₄H₂₁FN₈O₃

¹H-NMR (300 MHz, DMSO-d6) δ: 3.19-3.22 (m, 2H); 3.53-3.63 (m, 1H), 3.98-3.98 (m, 1H), 4.18-4.35 (m. 2H), 4.85 (s, 2H); 5.04-5.10 (m, 1H), 5.17-5.22 (m, 1H); 6.88 (s, 1H); 7.22 (s, 1H); 7.56-7.76 (m, 4H); 7.74 (s, 1H); 7.94-8.06 (m, 2H); 8.18 (s, 1H); 8.8 (s, 1H).

### Intermediate 35: 5-Bromo-2-[(5S)-5-(1H-imidazol-1-ylmethyl)-4,5-dihydroisoxazol-3-yl]pyridine

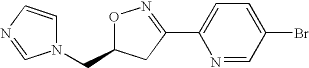

5-Bromo-2-[(5S)-5-(chloromethyl)-4,5-dihydroisoxazol-3-yl]pyridine (Intermediate 12, 300 mg, 1.09 mmol) was combined with imidazole (700 mg, 10.3 mmol), potassium carbonate (300 mg, 2.17 mmol), tetrabutylammonium iodide (5 mg, catalytic amount) and DMF (1 ml). The mixture was stirred 80° C. for 3 days, diluted with ethyl acetate, washed with water, dried over sodium sulfate, and evaporated. The material was purified by flash chromatography (silica gel, 0.5 to 5% methanol in dichloromethane) to give the title compound as a white solid (105 mg).

MS (electrospray): 308 (M+1) for C₁₂H₁₁BrN₄O

¹H-NMR (400 MHz DMSO-d₆) δ: 3.18 (dd, 1H); 3.52 (dd, 1H); 4.20 (dd, 1H); 4.27 (dd, 1H); 5.06 (m, 1H); 6.86 (s, 1H); 7.20 (s, 1H); 7.63 (s, 1H); 7.81 (d, 1H); 8.12 (dd, 1H); 8.77 (s, 1H).

### EXAMPLE 26a

### (5R)-3-(3-Fluoro-4-{6-[(5S)-5-(1H-imidazol-1-ylmethyl)-4,5-dihydroisoxazol-3-yl]pyridin-3-yl}phenyl)-5-(1H-1,2,3-triazol-1-ylmethyl)-1,3-oxazolidin-2-one hydrochloride salt

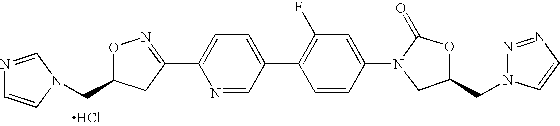

(5R)-3-(3-Fluoro-4-{6-[(5S)-5-(1H-imidazol-1-ylmethyl)-4,5-dihydroisoxazol-3-yl]pyridin-3-yl}phenyl)-5-(1H-1,2,3-triazol-1-ylmethyl)-1,3-oxazolidin-2-one (Example 26, 60 mg, 0.13 mmol) was dissolved in DMF:dioxane (1 ml:2 ml). 4M HCl in dioxane was added (0.035 ml, 0.14 mmol) at room temperature. The precipitate was filtered and washed with ether (1 ml) to yield the title compound as an off-white solid hydrochloride salt (40 mg).

MS (electrospray): 489 (MH⁺) for C₂₄H₂₁FN₈O₃

¹H-NMR (300 MHz, DMSO-d6) δ: 3.37-3.48 (m, 1H); 3.57 (s, 1H), 3.66-3.75 (m, 1H), 3.95-4.00 (m, 1H); 4.28-4.34 (m. 1H), 4.43-4.58 (m, 2H); 4.88 (m, 4H); 5.24 (m, 2H); 7.40-7.43 (m, 1H); 7.56-7.76 (m, 3H); 7.95-8.10 (m, 2H); 8.13 (s, 1H); 8.81 (s, 1H)

### EXAMPLE 27

### N-[2-({[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}amino)-2-oxoethyl]-N-methylglycine

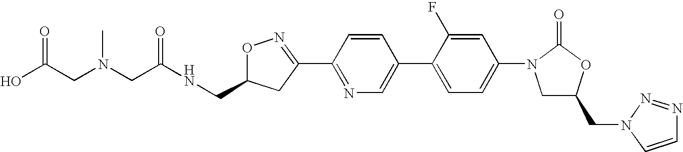

Methyl iminodiacetic acid (800 mg, 5.4 mmol) was dissolved in DMF (10 ml) and water (1 ml) followed by N-[3-(dimethylamino)propyl]-N′-ethylcarbodiimide?HCl (140 mg, 0.73 mmol). In a separate vial, (5R)-3-(4-{6-[(5S)-5-(aminomethyl)-4,5-dihydroisoxazol-3-yl]pyridin-3-yl}-3-fluorophenyl)-5-(1H-1,2,3-triazol-1-ylmethyl)-1,3-oxazolidin-2-one (Reference Example 16, 200 mg, 0.46 mmol) was dissolved in DMF (5 ml) while stirring under nitrogen, then diisopropyl ethyl amine (0.2 ml, 1.6 mmol) was added. The amine reaction mixture was added to the diacetic acid reaction mixture. The resulting mixture was allowed to stir for 1 hour at room temperature. The reaction mixture was diluted with ethyl acetate/water. The ethyl acetate layer was dried then concentrated to dryness. The oil obtained was purified by silica gel column. (elution with 100% dichloromethane to 100% methanol) (130 mg). The solid was further purified by reverse phase preparative chromatography (C18-0%-95% acetonitrile in water, 0.1% trifluoroacetic acid). The title compound was obtained as a yellow solid (73 mg) after drying at 40° C. under vacuum for 24 hours.

Melting point: 185-188° C.

MS (electrospray): 567 (M+1) for C₂₆H₂₇FN₈O₆

¹H-NMR (300 MHz, DMSO-d6) δ: 2.50 (m, 3H); 3.33-3.46 (m, 10H); 3.47-3.69 (m, 4H), 3.93-3.99 (m, 1H); 4.22-4.33 (m, 1H); 4.86-4.87 (m, 2H); 5.17-5.22 (m, 1H); 7.41-7.43 (dd, 1H); 7.56-7.72 (m, 1H); 7.76 (s, 1H); 7.98-8.08 (m, 1H); 8.19 (s, 1H); 8.43 (m, 1H); 8.82 (m, 1H).

### EXAMPLE 28

### N¹-{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-α-asparagine

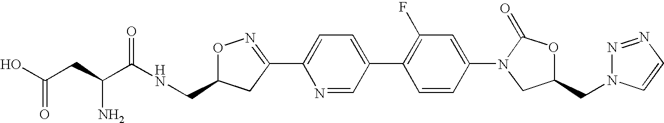

tert-Butyl N²-(tert-butoxycarbonyl)-N¹-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-α-asparaginate (Intermediate 36, 371 mg, 0.52 mmol) was dissolved in trifluoroacetic acid (5 ml) and stirred at room temperature for 1 hour. The solution was concentrated to dryness, dissolved in methanol (1 ml), then ether (15 ml) was added. The solid was collected, rinsed with ether and dried under vacuum to give the title product as an off-white solid (276 mg).

Melting point 224-226° C.

MS (electrospray): 553 (M+1) for C₂₅H₂₅FN₈O₆

¹H-NMR (300 MHz, DMSO-d6) δ 1.22-1.25 (m, 2H); 3.33-3.74 (m, 4H); 3.95 (m, 1H), 4.29 (m, 1H); 4.85 (m, 2H); 5.16 (m, 1H); 7.41-7.43 (dd, 1H); 7.54-7.74 (m, 2H); 7.76 (s, 1H); 7.98-8.08 (m, 3H); 8.19 (s, 1H); 8.32 (m, 1H); 8.82 (m, 1H).

### Intermediate 36: tert-Butyl N²-(tert-butoxycarbonyl)-N¹-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-α-asparaginate

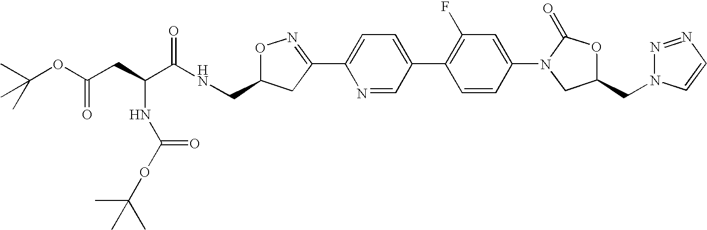

(5R)-3-(4-{6-[(5S)-5-(Aminomethyl)-4,5-dihydroisoxazol-3-yl]pyridin-3-yl}-3-fluorophenyl)-5-(1H-1,2,3-triazol-1-ylmethyl)-1,3-oxazolidin-2-one (Reference Example 16, 287 mg, 0.66 mmol), diisopropyl ethyl amine (0.345 ml, 1.98 mmol), N-[(dimethylamino)(3H-[1,2,3]triazolo[4,5-b]pyridin-3-yloxy)methylene]-N-methylmethanaminium hexafluorophosphate (300 mg, 0.79 mmol), N-tert-butoxycarbonyl-L-aspartic acid 4-tert-butyl ester (190 mg, 0.66 mmol) and DMF (3 ml) were combined and stirred at room temperature for 30 minutes. The reaction mixture was absorbed onto silica gel and was purified by silica gel column (elution with 100% dichloromethane to 10% methanol in dichloromethane). The title product was obtained as a yellow solid after drying at 40° C. under vacuum for 24 hours (371 mg).

MS (electrospray): 709 (M+1) for C₃₄H₁₁FN₈O₈

### EXAMPLE 29

### N¹-{[(5S)-3-(5-{2-Fluoro-4-[(SR)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-α-glutamine

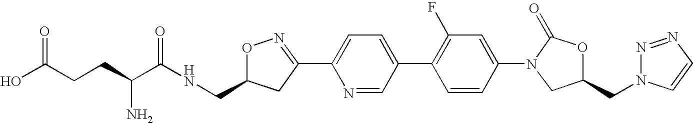

tert-Butyl N²-(tert-butoxycarbonyl)-N²-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-α-glutaminate (Intermediate 37, 500 mg, 0.69 mmol) was dissolved in trifluoroacetic acid (5 ml) and stirred at room temperature for 1 hour. The solution was concentrated to dryness, dissolved in methanol (1 ml), then ether (15 ml) was added. The solid was collected, rinsed with ether and dried under vacuum to give the title product as an off-white solid (276 mg).

Melting point 215.0-217.0° C.

MS (electrospray): 567 (M+1) for C₂₆H₂₇FN₈O₆

¹H-NMR (300 Mz. DMSO-d₆) δ: 1.22-1.25 (m, 3H); 3.33-3.74 (m, 8H); 3.95 (m, 1H), 4.29 (m, 1H, 4.85 (m, 3H); 5.16 (m, 1H); 7.41-7.43 (dd, 1H); 7.54-7.74 (m, 2H); 7.76 (s, 1H); 7.98-8.08 (m, 3H); 8.19 (s, 1H); 8.32 (m, 1H); 8.82 (m, 1H).

### Intermediate 37: tert-Butyl N²-(tert-butoxycarbonyl)-N¹-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-α-glutaminate

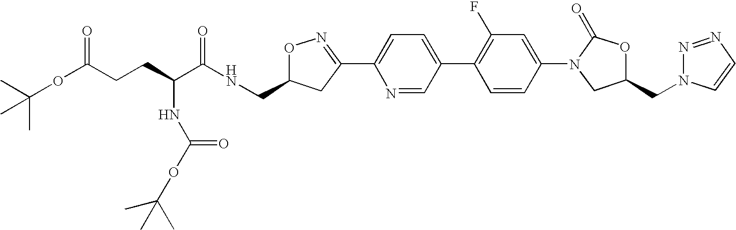

(5R)-3-(4-{6-[(SS)-5-(Aminomethyl)-4,5-dihydroisoxazol-3-yl]pyridin-3-yl}-3-fluorophenyl)-5-(1H-1,2,3-triazol-1-ylmethyl)-1,3-oxazolidin-2-one (Reference Example 16, 287 mg, 0.66 mmol), diisopropyl ethyl amine (0.345 ml, 1.98 mmol), N-[(dimethylamino)(3H-[1,2,3]triazolo[4,5-b]pyridin-3-yloxy)methylene]-N-methylmethanaminium hexafluorophosphate (300 mg, 0.79 mmol), N-tert-butoxycarbonyl-L-glutamic acid γ-tert-butyl ester (200 mg, 0.66 mmol) and DMF (3 ml) were combined and stirred at room temperature for 30 minutes. The reaction mixture was absorbed onto silica gel and was purified by silica gel column (elution with 100% dichloromethane to 10% methanol in dichloromethane). The title product (500 mg) was obtained as a yellow solid after drying at 40° C. under vacuum for 24 hours.

MS (electrospray): 723 (M+1) for C₃₅H₄₃FN₈O₈

1. A compound of the formula (I), or a pharmaceutically-acceptable salt, or pro-drug thereof, 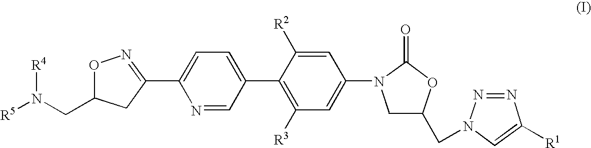
wherein:
R¹ is selected from hydrogen, halogen, cyano, methyl, cyanomethyl, fluoromethyl, difluoromethyl, trifluoromethyl, methylthio, and (2-4C)alkynyl;
R² and R³ are independently selected from hydrogen, fluoro, chloro and trifluoromethyl;
R⁴ and R⁵ are independently selected from hydrogen, allyl (optionally substituted on the carbon-carbon double bond by 1, 2 or 3 (1-4C)alkyl groups), methyl, cyanomethyl, carboxymethyl, —CH₂C(O)OR⁶, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, —NR⁶R⁷, —NHC(O)R⁶ and —NHS(O)₂R⁶], —C(O)R⁶, —C(O)CH₂NR⁶R⁷, —C(O)OR⁶, —C(O)NHR⁶, —C(O)NR⁶R⁷ and SO₂NHR⁶;
or R⁴ and R⁵ together with the nitrogen to which they are attached form a 5 or 6 membered, saturated or partially unsaturated heterocyclyl ring and optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl groups;
or R⁴ and R⁵ together with the nitrogen to which they are attached form an imidazole ring, which ring is optionally substituted on an available carbon by 1 or 2 methyl groups;
R⁶ and R⁷ are independently selected from hydrogen, methyl, cyclopropyl (optionally substituted with methyl), carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy);
or R⁶ or R⁷ may form a 4, 5 or 6 membered, carbon-linked saturated heterocyclyl ring, containing 1 or 2 heteroatoms independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom by 1 or 2 (1-4C)alkyl;
or R⁶ and R⁷ together with a nitrogen to which they are attached form a 4, 5 or 6 membered, saturated heterocyclyl ring, optionally containing 1 further heteroatom (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁶ and R⁷ are attached is not thereby quaternised) by 1 or 2 (1-4C)alkyl groups;
provided that R⁴ and R⁵ are not both hydrogen.

2. A compound of formula (I) or a pharmaceutically-acceptable salt, or pro-drug thereof 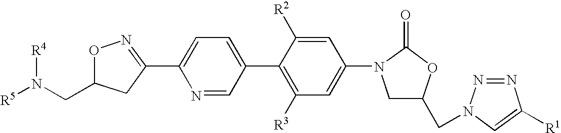
wherein
R¹ is selected from hydrogen, halogen, cyano, methyl, cyanomethyl, fluoromethyl, difluoromethyl, trifluoromethyl, methylthio, and (2-4C)alkynyl;
R² and R³ are independently selected from hydrogen, fluoro, chloro and trifluoromethyl;
R⁴ and R⁵ are independently selected from hydrogen, allyl (optionally substituted on the carbon-carbon double bond by 1, 2 or 3 (1-4C)alkyl groups), methyl, cyanomethyl, carboxymethyl, —CH₂C(O)OR⁶, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)R⁶, —S(O)₂NR⁶R⁷, —NR⁶R⁷, —NHC(O)R⁶ and —NHS(O)₂R⁶], —C(O)R⁶, —C(O)CH₂NR⁶R⁷, —C(O)OR⁶, —C(O)NHR⁶, —C(O)NR⁶R⁷ and —SO₂NHR⁶;
or R⁴ and R⁵ together with the nitrogen to which they are attached form a 5 or 6 membered, saturated or partially unsaturated heterocyclyl ring and optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl groups;
R⁶ and R⁷ are independently selected from hydrogen, methyl, cyclopropyl (optionally substituted with methyl), carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy);
or R⁶ or R⁷ may form a 4, 5 or 6 membered, carbon-linked saturated heterocyclyl ring, containing 1 or 2 heteroatoms independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom by 1 or 2 (1-4C)alkyl;
or R⁶ and R⁷ together with a nitrogen to which they are attached form a 4, 5 or 6 membered, saturated heterocyclyl ring, optionally containing 1 further heteroatom (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁶ and R⁷ are attached is not thereby quaternised) by 1 or 2 (1-4C)alkyl groups;
provided that R⁴ and R⁵ are not both hydrogen.

3. A compound of formula (I) or a pharmaceutically-acceptable salt, or prodrug thereof, as claimed in claim 1 or claim 2, wherein R¹ is selected from hydrogen, chloro, bromo, methyl and fluoromethyl.

4. A compound of formula (I) or a pharmaceutically-acceptable salt, or pro-drug thereof, as claimed in any one of claims 1 to 3, wherein R² and R³ are independently selected from hydrogen and fluoro.

5. A compound of formula (I) or a pharmaceutically-acceptable salt, or pro-drug thereof, as claimed in any one of claims 1 to 4, wherein R⁴ and R⁵ are independently selected from hydrogen, methyl, carboxymethyl, —CH₂C(O)OR⁶, —CH₂C(O)NR⁶R⁷, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶, —OC(O)R⁶, carboxy, —C(O)NR⁶R⁷, —S(O)₂R⁶, —S(O)₂NR⁶R⁷, NR⁶R⁷, —NHC(O)R⁶ and —NHS(O)₂R⁶], —C(O)R⁶ and —C(O)CH₂NR⁶R⁷.

6. A compound of formula (I) or a pharmaceutically-acceptable salt, or pro-drug thereof, as claimed in any one of claims 1 to 4, wherein R⁴ and R⁵ together with the nitrogen to which they are attached form a 5 or 6 membered, saturated or partially unsaturated heterocyclyl ring and optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl groups.

7. A compound of formula (I) or a pharmaceutically-acceptable salt, or pro-drug thereof, as claimed in any one of claims 1 to 6 wherein R⁶ and R⁷ are independently selected from hydrogen, methyl, cyclopropyl (optionally substituted with methyl), carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy).

8. A compound of formula (I) or a pharmaceutically-acceptable salt, or pro-drug thereof as claimed in any one of claims 1 to 7 which is a diastereomer of formula (Ia)

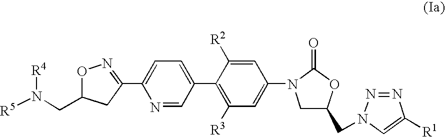

9. A compound of formula (I) or a pharmaceutically-acceptable salt, or pro-drug thereof as claimed in any one of claims 1 to 8 selected from:
(5R)-3-[4-(6-{(5S)-5-[(Dimethylamino)methyl]-4,5-dihydroisoxazol-3-yl}pyridin-3-yl)-3-fluorophenyl]-5-(1H-1,2,3-triazol-1-ylmethyl)-1,3-oxazolidin-2-one;
5R)-3-[3-Fluoro-4-(6-{(5S)-5-[(methylamino)methyl]-4,5-dihydroisoxazol-3-yl}pyridin-3-yl)phenyl]-5-(1H-1,2,3-triazol-1-ylmethyl)-1,3-oxazolidin-2-one;
(5R)-3-(3-Fluoro-4-{6-[(5S)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one;
(5R)-3-[3-Fluoro-4-(6-{(5S)-5-[(4-methylpiperazin-1-yl)methyl]-4,5-dihydroisoxazol-3-yl}pyridin-3-yl)phenyl]-5-(1H-1,2,3-triazol-1-ylmethyl)-1,3-oxazolidin-2-one;
5R)-3-{3-Fluoro-4-[6-((5S)-5-{[(2-hydroxyethyl)amino]methyl}-4,5-dihydroisoxazol-3-yl)pyridin-3-yl]phenyl}-5-(1H-1,2,3-triazol-1-ylmethyl)-1,3-oxazolidin-2-one;
(5R)-3-[4-(6-{(5S)-5-[(Butylamino)methyl]-4,5-dihydroisoxazol-3-yl}pyridin-3-yl)-3-fluorophenyl]-5-(1H-1,2,3-triazol-1-ylmethyl)-1,3-oxazolidin-2-one;
(5R)-3-(3-Fluoro-4-{6-[(5S)-5-(thiomorpholin-4-ylmethyl)-4,5-dihydroisoxazol-3-yl]pyridin-3-yl}phenyl)-5-(1H-1,2,3-triazol-1-ylmethyl)-1,3-oxazolidin-2-one;
(5R)-3-[3-Fluoro-4-(6-{(5S)-5-[(1-oxidothiomorpholin-4-yl)methyl]-4,5-dihydroisoxazol-3-yl}pyridin-3-yl)phenyl]-5-(1H-1,2,3-triazol-1-ylmethyl)-1,3-oxazolidin-2-one;
(5R)-3-[4-(6-{(5S)-5-[(1,1-Dioxidothiomorpholin-4-yl)methyl]-4,5-dihydroisoxazol-3-yl}pyridin-3-yl)-3-fluorophenyl]-5-(1H-1,2,3-triazol-1-ylmethyl)-1,3-oxazolidin-2-one;
(5R)-3-{3-Fluoro-4-[6-((5S)-5-{[(2-hydroxyethyl)(methyl)amino]methyl}-4,5-dihydroisoxazol-3-yl)pyridin-3-yl]phenyl}-5-(1H-1,2,3-triazol-1-ylmethyl)-1,3-oxazolidin-2-one;
(5R)-3-(3-Fluoro-4-{6-[(5R)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one;
(5R)-3-(3-Fluoro-4-{6-[(5S)-5-(pyrrolidin-1-ylmethyl)-4,5-dihydroisoxazol-3-yl]pyridin-3-yl}phenyl)-5-(1H-1,2,3-triazol-1-ylmethyl)-1,3-oxazolidin-2-one;
(5R)-3-(3-Fluoro-4-{6-[(5S)-5-(piperidin-1-ylmethyl)-4,5-dihydroisoxazol-3-yl]pyridin-3-yl}phenyl)-5-(1H-1,2,3-triazol-1-ylmethyl)-1,3-oxazolidin-2-one;
(5R)-3-(4-{6-[(5S)-5-(3,6-Dihydropyridin-1(2H)-ylmethyl)-4,5-dihydroisoxazol-3-yl]pyridin-3-yl}-3-fluorophenyl)-5-(1H-1,2,3-triazol-1-ylmethyl)-1,3-oxazolidin-2-one;
tert-Butyl {[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}carbamate;
N¹-{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-N²,N²-dimethylglycinamide;
tert-Butyl N-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-N-methylglycinate;
N-{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-N-methylglycine;
N-{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-prolinamide;
N¹-{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-D-valinamide;
N¹-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-alaninamide;
N¹-{[(5S)-3-(5-{2-fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-N¹,N²,N²-trimethylglycinamide;
N-{2-[{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}(methyl)amino]-2-oxoethyl}-N-methylglycine;
N-[2-({[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}amino)-2-oxoethyl]glycine;
(5R)-3-(3-Fluoro-4-{6-[(5S)-5-(1H-imidazol-1-ylmethyl)-4,5-dihydroisoxazol-3-yl]pyridin-3-yl}phenyl)-5-(1H-1,2,3-triazol-1-ylmethyl)-1,3-oxazolidin-2-one;
N-[2-({[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}amino)-2-oxoethyl]-N-methylglycine;
N¹-{[(5S)-3-(5-{2-Fluoro-4-[(SR)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-α-asparagine;
N¹-{[(5S)-3-(5-{2-Fluoro-4-[(5R)-2-oxo-5-(1H-1,2,3-triazol-1-ylmethyl)-1,3-oxazolidin-3-yl]phenyl}pyridin-2-yl)-4,5-dihydroisoxazol-5-yl]methyl}-L-α-glutamine

10. A method for producing an antibacterial effect in a warm blooded animal which comprises administering to said animal an effective amount of a compound of the invention as claimed in any one of claims 1 to 9, or a pharmaceutically-acceptable salt, or in-vivo hydrolysable ester thereof.

11. A compound of the invention as claimed in any one of claims 1 to 9, or a pharmaceutically-acceptable salt, or in-vivo hydrolysable ester thereof, for use as a medicament.

12. The use of a compound of the invention as claimed in any one of claims 1 to 9, or a pharmaceutically-acceptable salt, or in-vivo hydrolysable ester thereof, in the manufacture of a medicament for use in the production of an antibacterial effect in a warm blooded animal.

13. A pharmaceutical composition which comprises a compound of the invention as claimed in any one of claims 1 to 9, or a pharmaceutically-acceptable salt or an in-vivo hydrolysable ester thereof, and a pharmaceutically-acceptable diluent or carrier.

14. A pharmaceutical composition as claimed in claim 13, wherein said composition comprises a combination of a compound of the formula (I) and an antibacterial agent active against gram-positive bacteria.

15. A pharmaceutical composition as claimed in claim 14, wherein said composition comprises a combination of a compound of the formula (I) and an antibacterial agent active against gram-negative bacteria.

16. A process for the preparation of a compound of formula (I) as claimed in claim 1 or pharmaceutically acceptable salts or in-vivo hydrolysable esters thereof, which process comprises one of processes (a) to (j): and thereafter if necessary:
i) removing any protecting groups;
ii) forming a pro-drug (for example an in-vivo hydrolysable ester); and/or
iii) forming a pharmaceutically-acceptable salt;
wherein said processes (a) to (j) are as follows (wherein the variables are as defined in claim 1 unless otherwise stated):
a) by modifying a substituent in, or introducing a substituent into another compound of the invention;
b) by reaction of one part of a compound of formula (II) 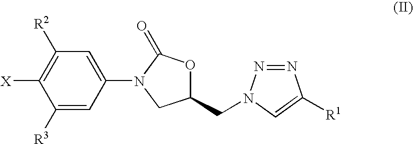
wherein X is a leaving group useful in palladium [0]coupling, with one part of a compound IIa, 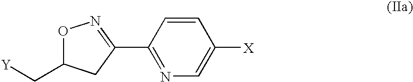
wherein Y is an amine or amine derivative NR⁴R⁵ as defined hereinbefore or hereinafter, a synthetic precursor thereof, or a protected derivative (PG=protecting group) thereof and X is a leaving group which may be the same or different from that in compound (II);
c) by reaction of a pyridyl-phenyl carbamate derivative (III) 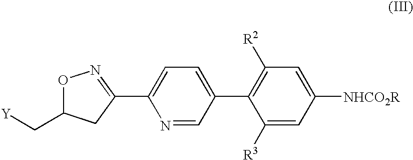
wherein Y is an amine or amine derivative NR⁴R⁵ as defined hereinbefore, with an appropriately substituted oxirane of formula 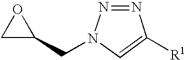
to form an oxazolidinone ring;
or by variations on this process in which the carbamate is replaced by an isocyanate or by an amine or/and in which the oxirane is replaced by an equivalent reagent X—CH₂CH(O-optionally protected)CH₂R₁a where X is a displaceable group;
(d) by reaction of a compound of formula (IV): 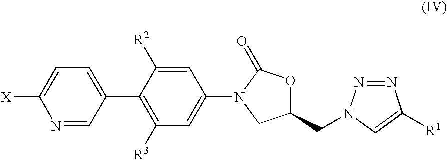
where X is a replaceable substituent with a compound of the formula (V): 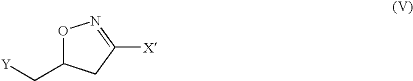
wherein X′ is a replaceable substituent and wherein Y is as hereinbefore defined; wherein the substituents X and X′ are chosen to be complementary pairs of substituents known in the art to be suitable as complementary substrates for coupling reactions catalysed by transition metals;
e) by reaction of an oxime of formula (VII) 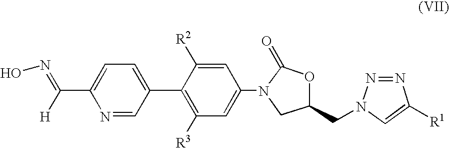
with a compound of formula 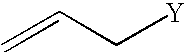
(wherein Y is as hereinbefore defined) to form an isoxazoline ring;
f) by formation of the triazole ring from a suitably functionalised intermediate in which the isoxazole-pyridyl-phenyl ring system is already formed;
g) by cycloaddition of an azide of formula 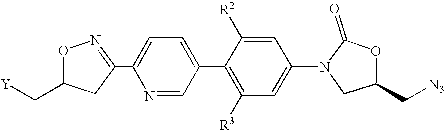
with an acetylene 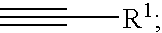
h) by reacting an aminomethyloxazolidinone of formula 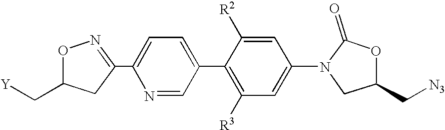
with an appropriate 1,1-dihaloketone sulfonylhydrazone;
i) for compounds of formula (I) wherein R¹ is halogen, by reacting an azidomethyl oxazolidinone of formula 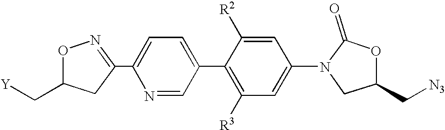
with an appropriate halovinylsulfonyl chloride;
j) by enantioselective esterase hydrolysis of a racemic mixture of esters of formula 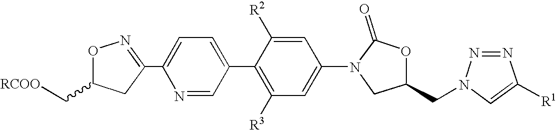
at the pro-chiral centre to give a hydroxyl group which can be converted into a NR⁴R⁵ substituent.

17. A compound of formula (IIa) 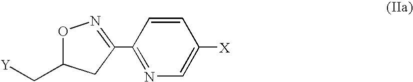
wherein either:
a) X is a boronic acid or ester and Y is NR⁴R⁵, wherein R⁴ and R⁵ are as defined for formula (I) in claim 1; or
b) X is halogen and Y is —OR⁴, wherein R⁴ is as defined for formula (I) in claim 1.
